(19) United States
(12) Patent Application Publication
(10) Pub. No.: US 2008/0221092 A1
(43) Pub. Date: Sep. 11, 2008

(54) Heterobicyclic metalloprotease inhibitors

(76) Inventors: Harald Bluhm, Dossenheim (DE); Matthias Hochgurtel, Schriesheim (DE); Heiko Kroth, Leimen (DE); Michael Essers, Bergisch Gladbach (DE); Christian Gege, Ehingen (DE); Frank Richter, Handschuhsheim (DE); Arthur Taveras, Southborough, MA (US)

Correspondence Address: AMGEN INC., MAIL STOP 28-2-C, ONE AMGEN CENTER DRIVE, THOUSAND OAKS, CA, 91320-1799, US

(21) Appl. No.: 11/986,586

(22) Filed: Nov. 20, 2007

Related U.S. Application Data
(60) Provisional application No. 60/860,155, filed on Nov. 20, 2006.

(51) Int. Cl. A61K 31/538; C07D 487/04; A61P 19/02; A61P 9/10; A61K 31/519

(52) U.S. Cl. 514/230.5; 544/280; 514/265.1; 544/105

(57) ABSTRACT

The present invention relates generally to amide containing heterobicyclic containing pharmaceutical agents, and in particular, to amide containing heterobicyclic metalloprotease inhibiting compounds. More particularly, the present invention provides a new class of heterobicyclic MMP-3 and/or MMP-13 inhibiting compounds that exhibit an increased potency and selectivity in relation to currently known MMP-13 and MMP-3 inhibitors.

This application claims the benefit of U.S. Provisional Application No. 60/860,155, filed Nov. 20, 2006, which is hereby incorporated by reference.

### FIELD OF THE INVENTION

The present invention relates generally to amide containing heterobicyclic metalloprotease inhibiting compounds and more particularly to heterobicyclic MMP-3 and/or MMP-13 inhibiting compounds.

### BACKGROUND OF THE INVENTION

Matrix metalloproteinases (MMPs) and aggrecanases (ADAMTS=a disintegrin and metalloproteinase with thrombopondin motif) are a family of structurally related zinc-containing enzymes that have been reported to mediate the breakdown of connective tissue in normal physiological processes such as embryonic development, reproduction, and tissue remodelling. Over-expression of MMPs and aggrecanases or an imbalance between extracellular matrix synthesis and degradation has been suggested as factors in inflammatory, malignant and degenerative disease processes. MMPs and aggrecanases are, therefore, targets for therapeutic inhibitors in several inflammatory, malignant and degenerative diseases such as rheumatoid arthritis, osteoarthritis, osteoporosis, periodontitis, multiple sclerosis, gingivitis, corneal epidermal and gastric ulceration, atherosclerosis, neointimal proliferation (which leads to restenosis and ischemic heart failure) and tumor metastasis.

The ADAMTSs are a group of proteases that are encoded in 19 ADAMTS genes in humans. The ADAMTSs are extracellular, multidomain enzymes whose functions include collagen processing, cleavage of the matrix proteoglycans, inhibition of angiogenesis and blood coagulation homoeostasis (Biochem. J. 2005, 386, 15-27; Arthritis Res. Ther. 2005, 7, 160-169; Curr. Med. Chem. Anti-Inflammatory Anti-Allergy Agents 2005, 4, 251-264).

The mammalian MMP family has been reported to include at least 20 enzymes, (Chem. Rev. 1999, 99, 2735-2776). Collagenase-3 (MMP-13) is among three collagenases that have been identified. Based on identification of domain structures for individual members of the MMP family, it has been determined that the catalytic domain of the MMPs contains two zinc atoms; one of these zinc atoms performs a catalytic function and is coordinated with three histidines contained within the conserved amino acid sequence of the catalytic domain. MMP-13 is over-expressed in rheumatoid arthritis, osteoarthritis, abdominal aortic aneurysm, breast carcinoma, squamous cell carcinomas of the head and neck, and vulvar squamous cell carcinoma. The principal substrates of MMP-13 are fibrillar collagens (types I, II, III) and gelatins, proteoglycans, cytokines and other components of ECM (extracellular matrix).

The activation of the MMPs involves the removal of a propeptide, which features an unpaired cysteine residue complexes the catalytic zinc (II) ion. X-ray crystal structures of the complex between MMP-3 catalytic domain and TIMP-1 and MMP-14 catalytic domain and TIMP-2 also reveal ligation of the catalytic zinc (II) ion by the thiol of a cysteine residue. The difficulty in developing effective MMP inhibiting compounds comprises several factors, including choice of selective versus broad-spectrum MMP inhibitors and rendering such compounds bioavailable via an oral route of administration.

MMP-3 (stromelysin-1; transin-1) is another member of the MMP family (Woesner; FASEB J. 1991; 5:2145-2154). Human MMP-3 was initially isolated from cultured human synoviocytes. It is also expressed by chondrocytes and has been localized in OA cartilage and synovial tissues (Case; Am. J. Pathol. 1989 December; 135(6):1055-64).

MMP-3 is produced by basal keratinocytes in a variety of chronic ulcers. MMP-3 mRNA and Protein were detected in basal keratinocytes adjacent to but distal from the wound edge in what probably represents the sites of proliferating epidermis. MMP-3 may this prevent the epidermis from healing (Saarialho-Kere, J. Clin. Invest. 1994 July; 94(1):79-88)).

MMP-3 serum protein levels are significantly elevated in patients with early and long-term rheumatoid arthritis (Yamanaka; Arthritis Rheum. 2000 April; 43(4):852-8) and in osteoarthritis patients (Bramono; Clin Orthop Relat Res. 2004 November; (428):272-85) as well as in other inflammatory diseases like systemic lupus erythematosis and ankylosing spondylitis (Chen, Rheumatology 2006 April; 45(4):414-20.).

MMP-3 acts on components of the ECM as aggrecan, fibronectin, gelatine, laminin, elastin, fibrillin and others and on collagens of type III, IV, V, VII, KX, X (Bramono; Clin Orthop Relat Res. 2004 November; (428):272-85). On collagens of type II and IX, MMP-3 exhibits telopeptidase activity (Sandell, Arthritis Res. 2001; 3(2):107-13; Eyre, Clin Orthop Relat Res. 2004 October; (427 Suppl):S118-22.). MMP-3 can activate other MMP family members as MMP-1; MMP-7; MMP-8; MMP-9 and MMP-13 (Close, Ann Rheum Dis 2001 November; 60 Suppl 3:iii62-7).

MMP-3 is involved in the regulation of cytokines and chemokines by releasing TGFβ1 from the ECM, activating TNFα, inactivation of IL-1β and release of IGF (Parks, Nat Rev Immunol. 2004 August; 4(8):617-29). A potential role for MMP-3 in the regulation of macrophate infiltration is based on the ability of the enzyme to converse active MCP species into antagonistic peptides (McQuibban, Blood. 2002 Aug. 15; 100(4):1160-7.).

### SUMMARY OF THE INVENTION

The present invention relates to a new class of heterobicyclic amide containing pharmaceutical agents which inhibits metalloproteases. In particular, the present invention provides a new class of metalloprotease inhibiting compounds that exhibit potent MMP-3 and/or MMP-13 inhibiting activity and/or activity towards MMP-8, MMP-12, ADAMTS-4, and ADAMTS-5.

The present invention provides several new classes of amide containing heterobicyclic metalloprotease compounds, of which some are represented by the following general formula:

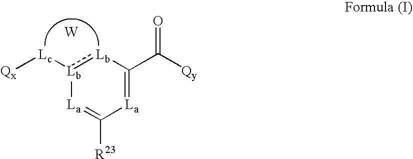

wherein all variables in the preceding Formula (I) are as defined herein below.

The heterobicyclic metalloprotease inhibiting compounds of the present invention may be used in the treatment of metalloprotease mediated diseases, such as rheumatoid arthritis, osteoarthritis, abdominal aortic aneurysm, cancer, inflammation, atherosclerosis, multiple sclerosis, chronic obstructive pulmonary disease, ocular diseases, neurological diseases, psychiatric diseases, thrombosis, bacterial infection, Parkinson's disease, fatigue, tremor, diabetic retinopathy, vascular diseases of the retina, aging, dementia, cardiomyopathy, renal tubular impairment, diabetes, psychosis, dyskinesia, pigmentary abnormalities, deafness, inflammatory and fibrotic syndromes, intestinal bowel syndrome, allergies, Alzheimer's disease, arterial plaque formation, periodontal, viral infection, stroke, cardiovascular disease, reperfusion injury, trauma, chemical exposure or oxidative damage to tissues, wound healing, hemorroid, skin beautifying, pain, inflammatory pain, bone pain and joint pain.

In particular, the heterobicyclic metalloprotease inhibiting compounds of the present invention may be used in the treatment of MMP-3 and/or MMP-13 mediated osteoarthritis and may be used for other MMP-3 and/or MMP-13 mediated symptoms, inflammatory, malignant and degenerative diseases characterized by excessive extracellular matrix degradation and/or remodelling, such as cancer, and chronic inflammatory diseases such as arthritis, rheumatoid arthritis, osteoarthritis atherosclerosis, abdominal aortic aneurysm, inflammation, multiple sclerosis, and chronic obstructive pulmonary disease, and pain, such as inflammatory pain, bone pain and joint pain.

The present invention also provides heterobicyclic metalloprotease inhibiting compounds that are useful as active ingredients in pharmaceutical compositions for treatment or prevention of MMP-3 and/or MMP-13 mediated diseases. The present invention also contemplates use of such compounds in pharmaceutical compositions for oral or parenteral administration, comprising one or more of the heterobicyclic metalloprotease inhibiting compounds disclosed herein.

The present invention further provides methods of inhibiting metalloproteases, by administering formulations, including, but not limited to, oral, rectal, topical, intravenous, parenteral (including, but not limited to, intramuscular, intravenous), ocular (ophthalmic), transdermal, inhalative (including, but not limited to, pulmonary, aerosol inhalation), nasal, sublingual, subcutaneous or intraarticular formulations, comprising the heterobicyclic metalloprotease inhibiting compounds by standard methods known in medical practice, for the treatment of diseases or symptoms arising from or associated with metalloprotease, especially MMP-13, including prophylactic and therapeutic treatment. Although the most suitable route in any given case will depend on the nature and severity of the conditions being treated and on the nature of the active ingredient. The compounds from this invention are conveniently presented in unit dosage form and prepared by any of the methods well-known in the art of pharmacy.

The heterobicyclic metalloprotease inhibiting compounds of the present invention may be used in combination with a disease modifying antirheumatic drug, a nonsteroidal anti-inflammatory drug, a COX-2 selective inhibitor, a COX-1 inhibitor, an immunosuppressive, a steroid, a biological response modifier or other anti-inflammatory agents or therapeutics useful for the treatment of chemokines mediated diseases.

### DETAILED DESCRIPTION OF THE INVENTION

One aspect of the present invention relates to a compound having the formula:

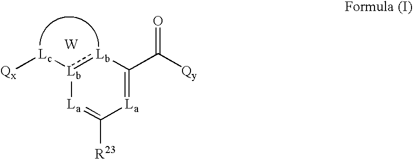

wherein:

R¹ in each occurrence is independently selected from hydrogen, alkyl, haloalkyl, trifluoroalkyl, alkenyl, alkynyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl,

wherein R¹ is optionally substituted one or more times, or

wherein R¹ is optionally substituted by one R¹⁶ group and optionally substituted by one or more R⁶ groups;

R² in each occurrence is selected from hydrogen and alkyl, wherein alkyl is optionally substituted one or more times or R¹ and R² when taken together with the nitrogen to which they are attached complete a 3- to 8-membered ring containing carbon atoms and optionally containing a heteroatom selected from O, S(O)ₓ, or NR⁵⁰ and which is optionally substituted one or more times;

R⁴ in each occurrence is independently selected from R¹⁰, hydrogen, alkyl, cycloalkyl, heterocycloalkyl, aryl, heteroaryl, halo, haloalkyl, CF₃, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, (C₀-C₆)-alkyl-C(O)—NR¹¹—CN, O—(C₀-C₆)-alkyl-C(O)NR¹⁰R¹¹, S(O)ₓ—(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹⁰, O—(C₀-C₆)-alkyl-aryl and O—(C₀-C₆)-alkyl-heteroaryl,

wherein each R⁴ group is optionally substituted one or more times, or

wherein each R⁴ group is optionally substituted by one or more R¹⁴ groups;

R⁵ in each occurrence is independently selected from hydrogen, alkyl, C(O)NR¹⁰R¹¹, aryl, arylalkyl, SO₂NR¹⁰R¹¹ and C(O)OR¹⁰, wherein alkyl, aryl and arylalkyl are optionally substituted one or more times;

R⁶ is independently selected from R⁹, alkenyl, alkynyl, cycloalkyl, heterocycloalkyl, bicycloalkyl, heterobicycloalkyl, spiroalkyl, spiroheteroalkyl, aryl, heteroaryl, C(O)OR¹⁰, CH(CH₃)CO₂H, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-P(O)₂OH, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-NR¹⁰C(═N—CN)NR¹⁰R¹¹, (C₀-C₆)-alkyl-C(═N—CN)NR¹⁰R¹¹, (C₀-C₆)-alkyl-NR¹⁰C(═N—NO₂)NR¹⁰R¹¹, (C₀-C₆)-alkyl-C(═N—NO₂)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, C(O)NR¹⁰—(C₀-C₆)-alkyl-heteroaryl, C(O)NR¹⁰—(C₀-C₆)-alkyl-aryl, S(O)₂NR¹⁰—(C₀-C₆)-alkyl-aryl, S(O)₂NR¹⁰—(C₀-C₆)-alkyl-heteroaryl, S(O)₂NR¹⁰-alkyl, S(O)₂—(C₀-C₆)-alkyl-aryl, S(O)₂—(C₀-C₆)-alkyl-heteroaryl, (C₀-C₆)-alkyl-C(O)—NR¹¹—CN, O—(C₀-C₆)-alkyl-C(O)NR¹⁰R¹¹, S(O)ₓ—(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹¹, O—(C₀-C₆)-alkyl-aryl and O—(C₀-C₆)-alkyl-heteroaryl,

wherein each R⁶ group is optionally substituted one or more times, or

wherein each R⁶ group is optionally substituted by one or more R¹⁴ groups;

R⁹ in each occurrence is independently selected from R¹⁰, hydrogen, alkyl, cycloalkyl, heterocycloalkyl, aryl, heteroaryl, halo, CHF₂, CF₃, OR¹⁰, SR¹⁰, COOR¹⁰, CH(CH₃)CO₂H, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-P(O)₂OH, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-NR¹⁰C(═N—CN)NR¹⁰R¹¹, (C₀-C₆)-alkyl-C(═N—CN)NR¹⁰R¹¹, (C₀-C₆)-alkyl-NR¹⁰C(═N—NO₂)NR¹⁰R¹¹, (C₀-C₆)-alkyl-C(═N—NO₂)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, C(O)NR¹⁰—(C₀-C₆)-alkyl-heteroaryl, C(O)NR¹⁰—(C₀-C₆)-alkyl-aryl, S(O)₂NR¹⁰—(C₀-C₆)-alkyl-aryl, S(O)₂NR¹⁰—(C₀-C₆)-alkyl-heteroaryl, S(O)₂NR¹⁰-alkyl, S(O)₂—(C₀-C₆)-alkyl-aryl, S(O)₂—(C₀-C₆)-alkyl-heteroaryl, (C₀-C₆)-alkyl-C(O)—NR¹⁰—CN, O—(C₀-C₆)-alkyl-C(O)NR¹⁰R¹¹, S(O), —(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹¹, O—(C₀-C₆)-alkyl-aryl and O—(C₀-C₆)-alkyl-heteroaryl,

wherein each R⁹ group is optionally substituted, or

wherein each R⁹ group is optionally substituted by one or more R¹⁴ groups;

R¹⁰ and R¹¹ in each occurrence are independently selected from hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted, or R¹⁰ and R¹¹ when taken together with the nitrogen to which they are attached complete a 3- to 8-membered ring containing carbon atoms and optionally containing a heteroatom selected from O, S(O)ₓ, or NR⁵⁰ and which is optionally substituted;

R¹⁴ is independently selected from hydrogen, alkyl, arylalkyl, cycloalkylalkyl, heteroarylalkyl, heterocyclylalkyl and halo, wherein alkyl, arylalkyl, cycloalkylalkyl, heteroarylalkyl and heterocyclylalkyl are optionally substituted one or more times.

R¹⁶ is selected from cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, heterocycloalkyl fused heteroarylalkyl, (i) and (ii):

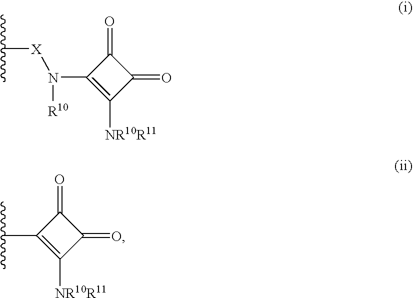

wherein cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl are optionally substituted one or more times;

R²⁰ is selected from hydrogen and alkyl, wherein alkyl is optionally substituted;

R²¹ is a bicyclic or tricyclic fused ring system, wherein at least one ring is partially saturated, and

wherein R²¹ is optionally substituted one or more times, or

wherein R²¹ is optionally substituted by one or more R⁹ groups;

R²³ is selected from hydrogen, hydroxy, halo, alkyl, cycloalkyl, alkoxy, alkenyl, alkynyl, aryl, heteroaryl, NO₂, NR¹⁰R¹¹, CN, SR¹⁰, SSR¹⁰, PO₃R¹⁰, NR¹⁰NR¹⁰R¹¹, NR¹⁰N═CR¹⁰R¹¹, NR¹⁰SO₂R¹¹, C(O)NR¹⁰R¹¹, C(O)OR¹⁰, and fluoroalkyl, wherein alkyl, cycloalkyl, alkoxy, alkenyl, alkynyl, and fluoroalkyl are optionally substituted one or more times;

R³⁰ is selected from alkyl and (C₀-C₆)-alkyl-aryl, wherein alkyl and aryl are optionally substituted;

R⁵⁰ in each occurrence is independently selected from hydrogen, alkyl, aryl, heteroaryl, C(O)R⁸⁰, C(O)NR⁸⁰R⁸¹, SO₂R⁸⁰ and SO₂NR⁸⁰R⁸¹, wherein alkyl, aryl, heteroaryl, C(O)R⁸⁰, C(O)NR⁸⁰R⁸¹, SO₂R⁸⁰ and SO₂NR⁸⁰R⁸¹ are optionally substituted;

R⁸⁰ and R⁸¹ in each occurrence are independently selected from hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted, or R⁸⁰ and R⁸¹ when taken together with the nitrogen to which they are attached complete a 3- to 8-membered ring containing carbon atoms and optionally a heteroatom selected from O, S(O)ₓ, —NH, and —N(alkyl) and which is optionally substituted;

E is selected from a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—, —CH₂—W¹— and

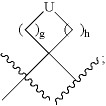

Lₐ is independently selected from CR⁹ and N;

L_{b} is independently selected from C and N with the provisos that both L_{b} are not N, and that the bond between L_{b} and L_{b} is optionally a double bond only if both are L_{b} are carbon;

L_{c} is selected from C and N;

Qₓ is absent or selected from R¹, NO₂, CN, haloalkyl, halogen, SO₂NR¹R², S(O)ₓR¹, SO₃H, C(O)OR¹, NR¹SO₂R¹, OC(O)R¹, OC(O)NR¹R², NR¹CO₂R¹, NR¹C(O)NR¹R², NR¹C(═NR¹), OR¹, OR²¹ and

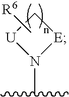

Q_{y} is selected from NR¹R², NR²¹R²¹ and OR¹;

W is a 5- or 6-membered ring selected from cycloalkyl, heterocycloalkyl, aryl and heteroaryl, wherein cycloalkyl, heterocycloalkyl, aryl and heteroaryl are optionally substituted one or more times with R⁴;

U is selected from C(R⁵R¹⁰), NR⁵, O, S, S═O and S(═O)₂;

W¹ is selected from O, NR⁵, S, S═O, S(═O)₂, N(R¹⁰)(C═O), N(R¹⁰)S(═O)₂ and S(═O)₂N(R¹⁰);

X is selected from a bond and (CR¹⁰R¹¹)_{w}E(CR¹⁰R¹¹)_{w};

g and h are independently selected from 0-2;

n is selected from 0-3;

w is independently selected from 0-4;

x is selected from 0 to 2;

y is selected from 1 and 2;

the dotted line optionally represents a double bond; and

N-oxides, pharmaceutically acceptable salts, prodrugs, formulation, polymorphs, tautomers, racemic mixtures and stereoisomers thereof.

In one embodiment, in conjunction with any above or below embodiments:

R¹ in each occurrence is independently selected from hydrogen, alkyl, haloalkyl, trifluoroalkyl, alkenyl, alkynyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl,

wherein R¹ is optionally substituted one or more times, or

wherein R¹ is optionally substituted by one R¹⁶ group and optionally substituted by one or more R⁶ groups;

R² in each occurrence is selected from hydrogen and alkyl, wherein alkyl is optionally substituted one or more times or R¹ and R² when taken together with the nitrogen to which they are attached complete a 3- to 8-membered ring containing carbon atoms and optionally containing a heteroatom selected from O, S(O)ₓ, or NR⁵⁰ and which is optionally substituted one or more times;

R⁴ in each occurrence is independently selected from R¹⁰, hydrogen, alkyl, cycloalkyl, heterocycloalkyl, aryl, heteroaryl, halo, haloalkyl, CF₃, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-S(O)_{y}NR¹⁰R¹¹, (C₀-C₆)-alkyl-NR¹⁰CONR¹¹SO₂R³, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, (C₀-C₆)-alkyl-C(O)—NR¹¹—CN, O—(C₀-C₆)-alkyl-C(O)NR¹⁰R¹¹, S(O), —(C₀-C₆)-alkyl-C(O)OR¹⁰, S(O), —(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹⁰, O—(C₀-C₆)-alkyl-aryl and O—(C₀-C₆)-alkyl-heteroaryl,

wherein each R⁴ group is optionally substituted one or more times, or

wherein each R⁴ group is optionally substituted by one or more R¹⁴ groups;

R⁵ in each occurrence is independently selected from hydrogen, alkyl, C(O)NR¹⁰R¹¹, aryl, arylalkyl, SO₂NR¹⁰R¹¹ and C(O)OR¹⁰, wherein alkyl, aryl and arylalkyl are optionally substituted one or more times;

R⁶ is independently selected from R⁹, alkenyl, alkynyl, cycloalkyl, heterocycloalkyl, bicycloalkyl, heterobicycloalkyl, spiroalkyl, spiroheteroalkyl, aryl, heteroaryl, C(O)OR¹⁰, CH(CH₃)CO₂H, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-P(O)₂OH, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-NR¹⁰C(═N—CN)NR¹⁰R¹¹, (C₀-C₆)-alkyl-C(═N—CN)NR¹⁰R¹¹, (C₀-C₆)-alkyl-NR¹⁰C(═N—NO₂)NR¹⁰R¹¹, (C₀-C₆)-alkyl-C(═N—NO₂)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, C(O)NR¹⁰—(C₀-C₆)-alkyl-heteroaryl, C(O)NR¹⁰—(C₀-C₆)-alkyl-aryl, S(O)₂NR¹⁰—(C₀-C₆)-alkyl-aryl, S(O)₂NR¹⁰—(C₀-C₆)-alkyl-heteroaryl, S(O)₂NR¹⁰-alkyl, S(O)₂—(C₀-C₆)-alkyl-aryl, S(O)₂—(C₀-C₆)-alkyl-heteroaryl, (C₀-C₆)-alkyl-C(O)—NR¹¹—CN, O—(C₀-C₆)-alkyl-C(O)NR¹⁰R¹¹, S(O), —(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹¹, O—(C₀-C₆)-alkyl-aryl and O—(C₀-C₆)-alkyl-heteroaryl,

wherein each R⁶ group is optionally substituted one or more times, or

wherein each R⁶ group is optionally substituted by one or more R¹⁴ groups;

R⁹ in each occurrence is independently selected from R¹⁰, hydrogen, alkyl, cycloalkyl, heterocycloalkyl, aryl, heteroaryl, halo, CHF₂, CF₃, OR¹⁰, SR¹⁰, COOR¹⁰, CH(CH₃)CO₂H, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-P(O)₂OH, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-NR¹⁰C(═N—CN)NR¹⁰R¹¹, (C₀-C₆)-alkyl-C(═N—CN)NR¹⁰R¹¹, (C₀-C₆)-alkyl-NR¹⁰C(═N—NO₂)NR¹⁰R¹¹, (C₀-C₆)-alkyl-C(═N—NO₂)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, C(O)NR¹⁰—(C₀-C₆)-alkyl-heteroaryl, C(O)NR¹⁰—(C₀-C₆)-alkyl-aryl, S(O)₂NR¹⁰—(C₀-C₆)-alkyl-aryl, S(O)₂NR¹⁰—(C₀-C₆)-alkyl-heteroaryl, S(O)₂NR¹⁰-alkyl, S(O)₂—(C₀-C₆)-alkyl-aryl, S(O)₂—(C₀-C₆)-alkyl-heteroaryl, (C₀-C₆)-alkyl-C(O)—NR¹⁰—CN, O—(C₀-C₆)-alkyl-C(O)NR¹⁰R¹¹, S(O), —(C₀-C₆)-alkyl-C(O)OR¹⁰, S(O), —(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹¹, O—(C₀-C₆)-alkyl-aryl and O—(C₀-C₆)-alkyl-heteroaryl,

wherein each R⁹ group is optionally substituted, or

wherein each R⁹ group is optionally substituted by one or more R¹⁴ groups;

R¹⁰ and R¹¹ in each occurrence are independently selected from hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted, or R¹⁰ and R¹¹ when taken together with the nitrogen to which they are attached complete a 3- to 8-membered ring containing carbon atoms and optionally containing a heteroatom selected from O, S(O)ₓ, or NR⁵⁰ and which is optionally substituted;

R¹⁴ is independently selected from hydrogen, alkyl, arylalkyl, cycloalkylalkyl, heteroarylalkyl, heterocyclylalkyl and halo, wherein alkyl, arylalkyl, cycloalkylalkyl, heteroarylalkyl and heterocyclylalkyl are optionally substituted one or more times.

R¹⁶ is selected from cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, heterocycloalkyl fused heteroarylalkyl, (i) and (ii):

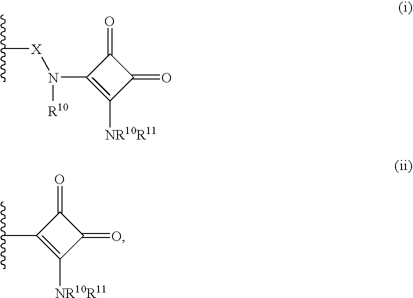

wherein cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl are optionally substituted one or more times;

R²⁰ is selected from hydrogen and alkyl, wherein alkyl is optionally substituted;

R²¹ is a bicyclic or tricyclic fused ring system, wherein at least one ring is partially saturated, and

wherein R²¹ is optionally substituted one or more times, or

wherein R²¹ is optionally substituted by one or more R⁹ groups;

R²³ is selected from hydrogen, hydroxy, halo, alkyl, cycloalkyl, alkoxy, alkenyl, alkynyl, aryl, heteroaryl, NO₂, NR¹⁰R¹¹, CN, SR¹⁰, SSR¹⁰, PO₃R¹⁰, NR¹⁰NR¹⁰R¹¹, NR¹⁰N═CR¹⁰R¹¹, NR¹⁰SO₂R¹¹, C(O)NR¹⁰R¹¹, C(O)OR¹⁰, and fluoroalkyl, wherein alkyl, cycloalkyl, alkoxy, alkenyl, alkynyl, and fluoroalkyl are optionally substituted one or more times;

R³⁰ is selected from alkyl and (C₀-C₆)-alkyl-aryl, wherein alkyl and aryl are optionally substituted;

R⁵⁰ in each occurrence is independently selected from hydrogen, alkyl, aryl, heteroaryl, C(O)R⁸⁰, C(O)NR⁸⁰R⁸¹, SO₂R⁸⁰ and SO₂NR⁸⁰R⁸¹, wherein alkyl, aryl, heteroaryl, C(O)R⁸⁰, C(O)NR⁸⁰R⁸¹, SO₂R⁸¹ and SO₂NR⁸⁰R⁸¹ are optionally substituted;

R⁸⁰ and R⁸¹ in each occurrence are independently selected from hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted, or R⁸⁰ and R⁸¹ when taken together with the nitrogen to which they are attached complete a 3- to 8-membered ring containing carbon atoms and optionally a heteroatom selected from O, S(O)ₓ, —NH, and —N(alkyl) and which is optionally substituted;

E is selected from a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—, —CH₂—W¹— and

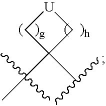

Lₐ is independently selected from CR⁹ and N;

L_{b} is independently selected from C and N with the provisos that both L_{b} are not N, and that the bond between L_{b} and L_{b} is optionally a double bond only if both are L_{b} are carbon;

L_{c} is selected from C and N;

Qₓ is selected from NO₂, CN, SO₂NR¹R², S(O)ₓR¹, SO₃H, C(O)OR¹, NR¹SO₂R¹, OC(O)R¹, OR¹, OR²¹ and

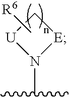

Q_{y} is selected from NR¹R², NR²⁰R²¹ and OR¹;

W is a 5- or 6-membered ring selected from cycloalkyl, heterocycloalkyl, aryl and heteroaryl, wherein cycloalkyl, heterocycloalkyl, aryl and heteroaryl are optionally substituted one or more times with R⁴;

U is selected from C(R⁵R¹⁰), NR⁵, O, S, S═O and S(═O)₂;

W¹ is selected from O, NR⁵, S, S═O, S(═O)₂, N(R¹⁰)(C═O), N(R¹⁰)S(═O)₂ and S(═O)₂N(R¹⁰);

X is selected from a bond and (CR¹⁰R¹¹)_{w}E(CR¹⁰R¹¹)_{w};

g and h are independently selected from 0-2;

n is selected from 0-3;

w is independently selected from 0-4;

x is selected from 0 to 2;

y is selected from 1 and 2;

the dotted line optionally represents a double bond; and

N-oxides, pharmaceutically acceptable salts, prodrugs, formulation, polymorphs, tautomers, racemic mixtures and stereoisomers thereof.

In another embodiment, in conjunction any above or below embodiments, Lₐ is N.

In another embodiment, in conjunction any above or below embodiments, L_{b} is C.

In another embodiment, in conjunction any above or below embodiments, L_{c} is C.

In another embodiment, in conjunction any above or below embodiments, Qₓ SO₂NR¹R², S(O)ₓR¹, SO₃H, or NR¹SO₂R¹.

In another embodiment, in conjunction any above or below embodiments, Qₓ is NR¹SO₂R¹.

In another embodiment, in conjunction any above or below embodiments, Qₓ is C(O)OR¹, OC(O)R¹, or OR¹.

In another embodiment, in conjunction any above or below embodiments, Qₓ is

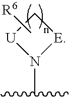

In another embodiment, in conjunction any above or below embodiments, the compound is selected from:

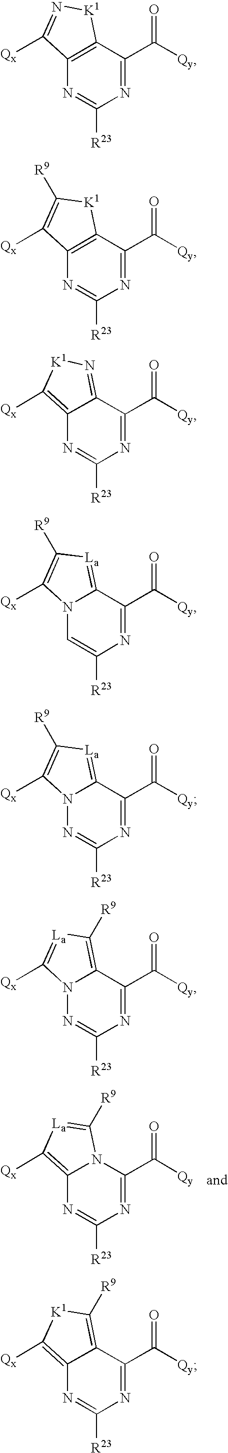

wherein:

Q_{y} is selected from NR¹R² and NR²⁰R²¹;

K¹ is O, S(O)ₓ, or NR⁵¹; and

R⁵¹ is independently selected from hydrogen, alkyl, aryl, heteroaryl, arylalkyl, cycloalkylalkyl, heteroarylalkyl and haloalkyl, wherein alkyl, aryl, heteroaryl, arylalkyl, cycloalkylalkyl, heteroarylalkyl and haloalkyl are optionally substituted one or more times.

In another embodiment, in conjunction any above or below embodiments, the compound is selected from:

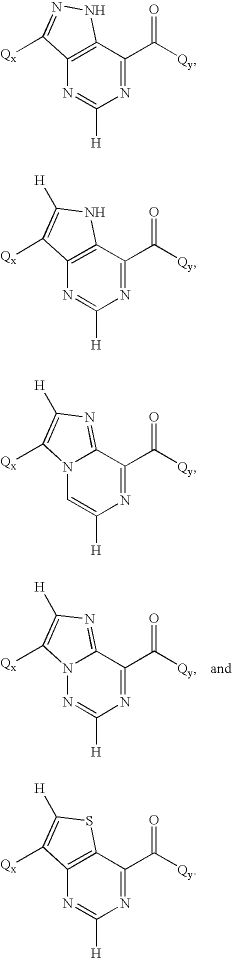

In another embodiment, in conjunction any above or below embodiments,

Qₓ=SO₂NR¹R², SO₂NR²R², S(O)ₓR¹, C(O)OR¹, NR²SO₂R¹, OC(O)R¹ and OR¹; and

the R¹ in Q_{y} is selected from:

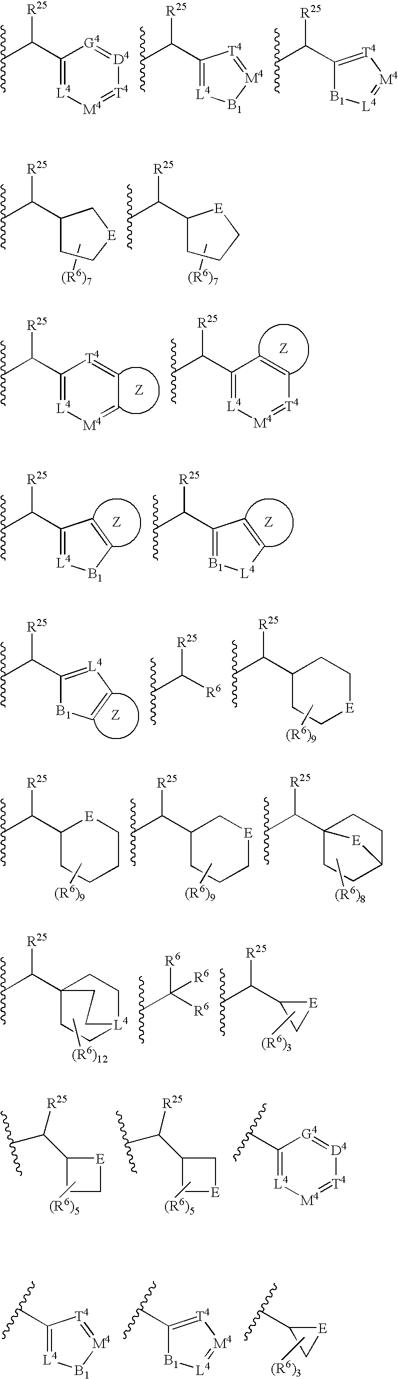

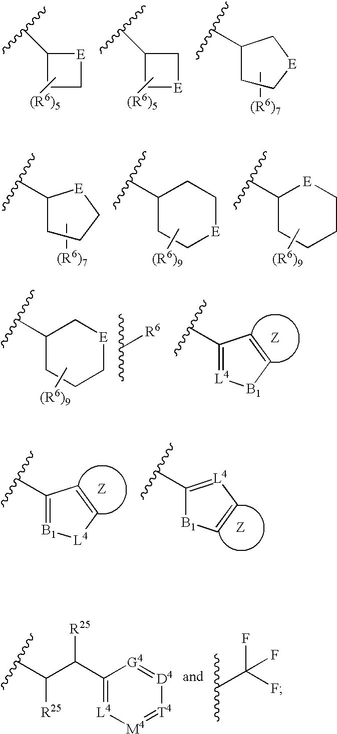

wherein:

R⁹ is independently selected from hydrogen, alkyl, halo, CHF₂, CF₃, OR¹⁰, NR¹⁰R¹¹, NO₂, and CN, wherein alkyl is optionally substituted one or more times;

R²⁵ is independently selected from hydrogen, alkyl, cycloalkyl, C(O)R¹⁰, C(O)NR¹⁰R¹¹ and haloalkyl, wherein alkyl, cycloalkyl, and haloalkyl are optionally substituted one or more times;

B₁ is selected from the group consisting of NR¹⁰, O and S(O)ₓ;

D⁴, G⁴, L⁴, M⁴, and T⁴, are independently selected from CR⁶ and N;

Z is a 5- to 8-membered ring consisting of cycloalkyl, heterocycloalkyl, aryl and heteroaryl, wherein cycloalkyl, heterocycloalkyl, aryl and heteroaryl are optionally substituted one or more times.

In another embodiment, in conjunction any above or below embodiments, Qₓ=SO₂NR¹R², SO₂NR²R², S(O)ₓR¹, NR²SO₂R¹; and the R¹ in Q_{y} is selected from

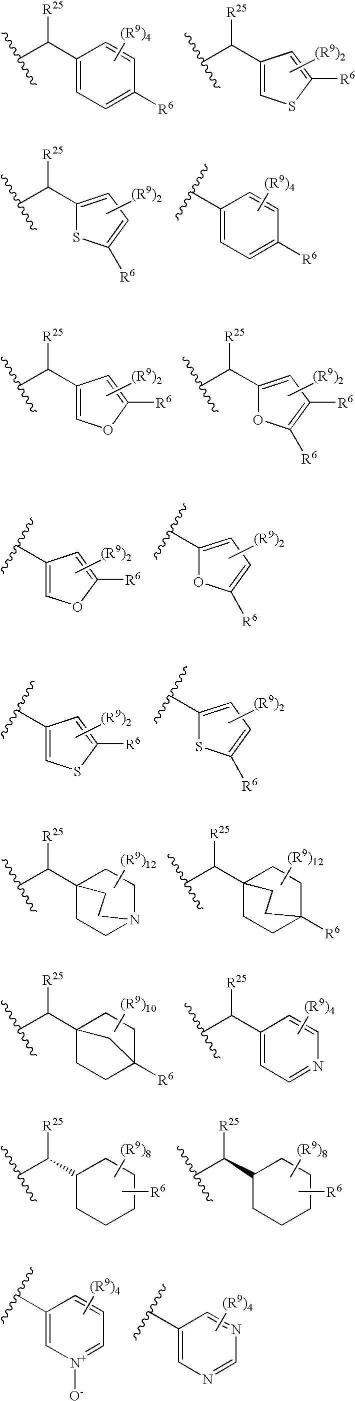

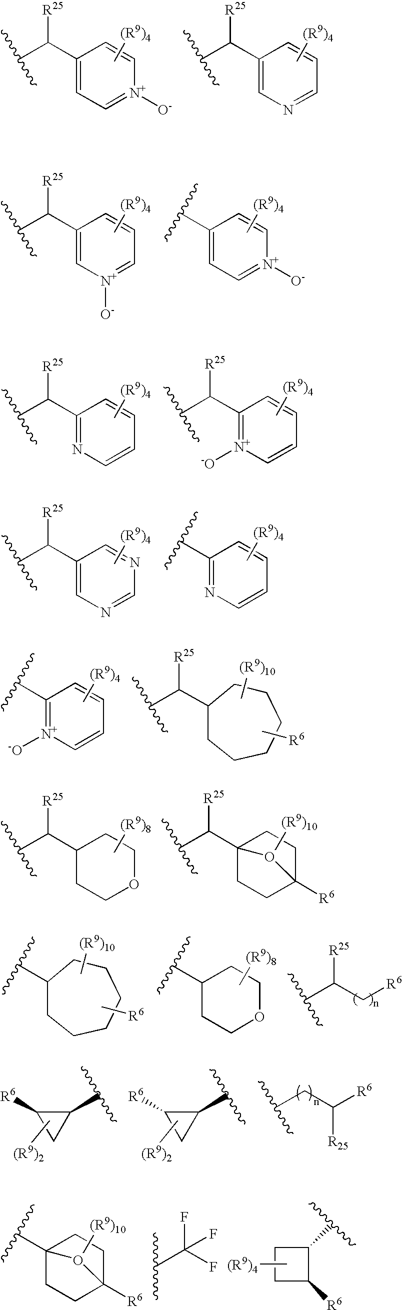

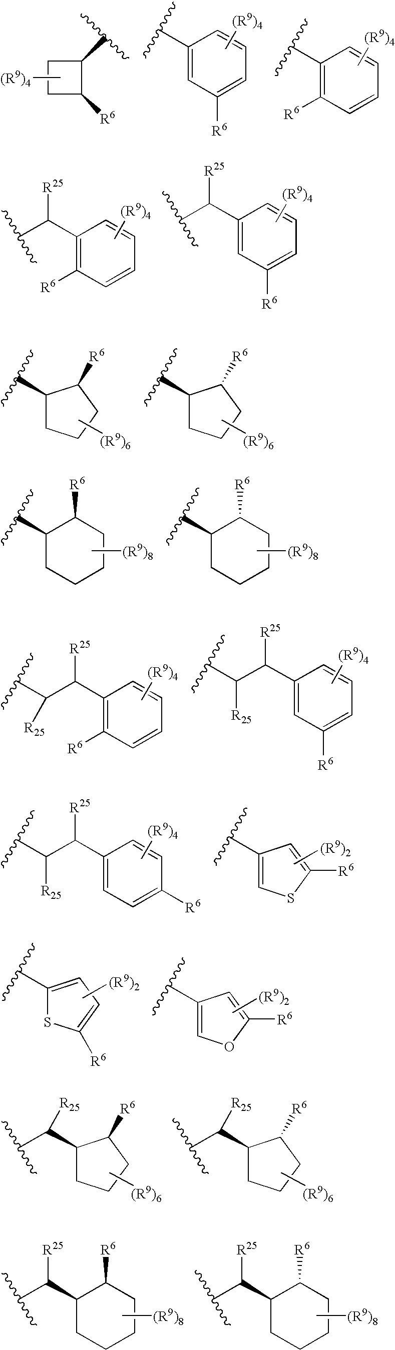

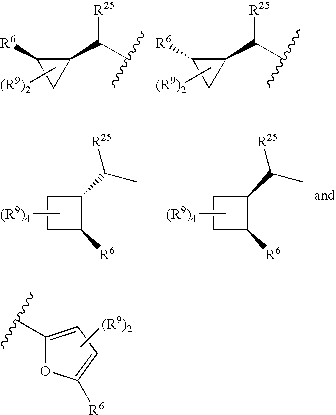

In another embodiment, in conjunction any above or below embodiments, R⁶ is selected from hydrogen, halo, CN, OH, CH₂OH, CF₃, CHF₂, OCF₃, OCHF₂, SO₂CH₃, SO₂CF₃, SO₂NH₂, SO₂NHCH₃, SO₂N(CH₃)₂, NH₂, NHCOCH₃, NHCONH₂, NHSO₂CH₃, alkoxy, alkyl, alkynyl, CO₂H,

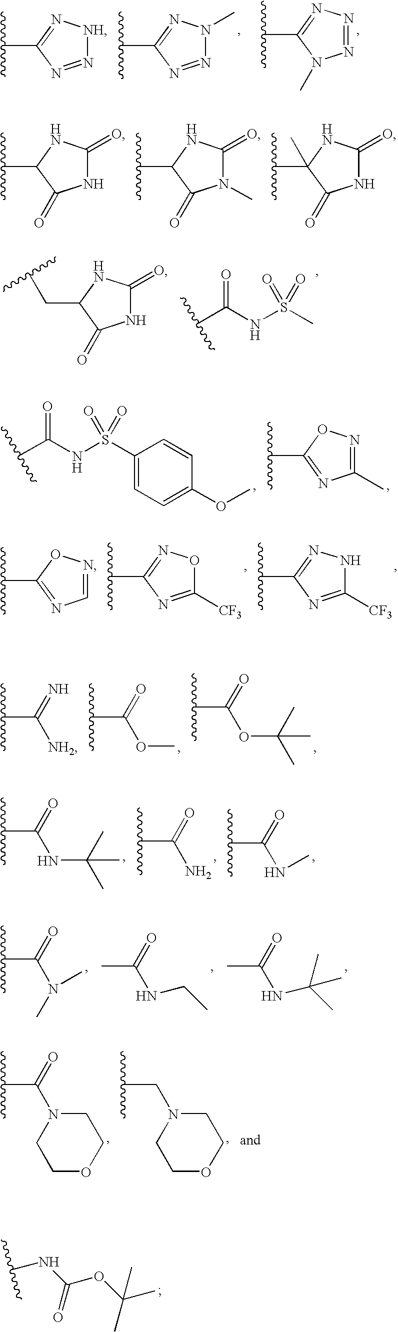

R⁹ is independently selected from hydrogen, fluoro, chloro, CH₃, CF₃, CHF₂, OCF₃, OCH₃ and OCHF₂; and

R²⁵ is selected of hydrogen, CH₃, COOMe, COOH, CONH₂, CONHMe and CON(Me)₂.

In another embodiment, in conjunction any above or below embodiments, Qₓ=SO₂NR¹R², SO₂NR²R², S(O)ₓR¹, C(O)OR¹, NR²SO₂R¹, OC(O)R¹ and OR¹; and

the R¹ in Q_{y} is selected from:

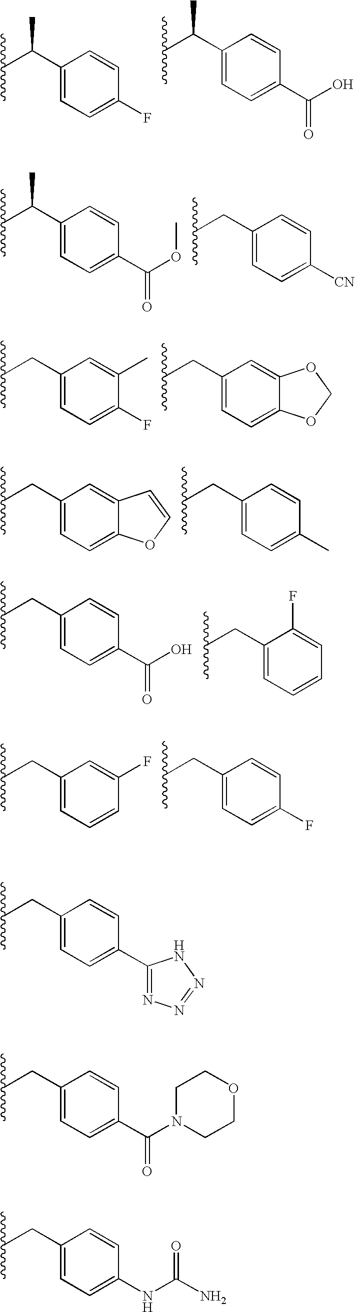

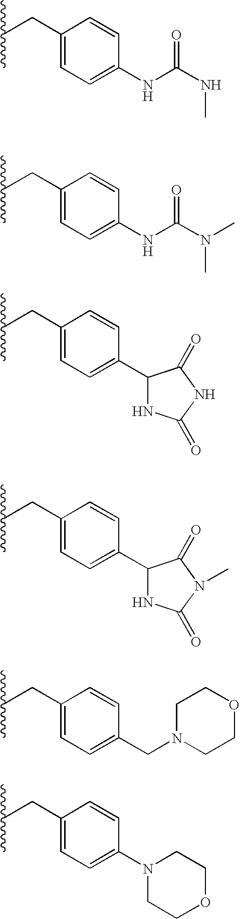

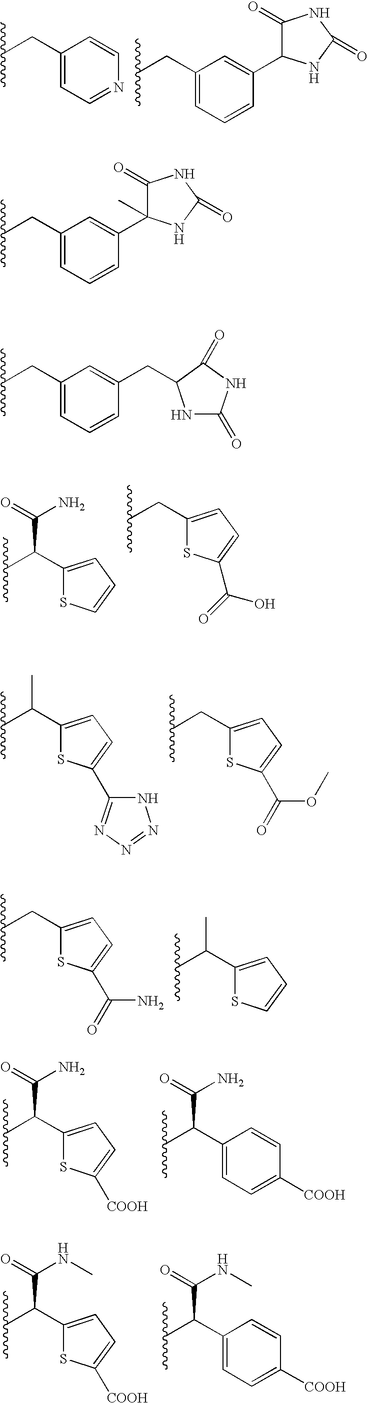

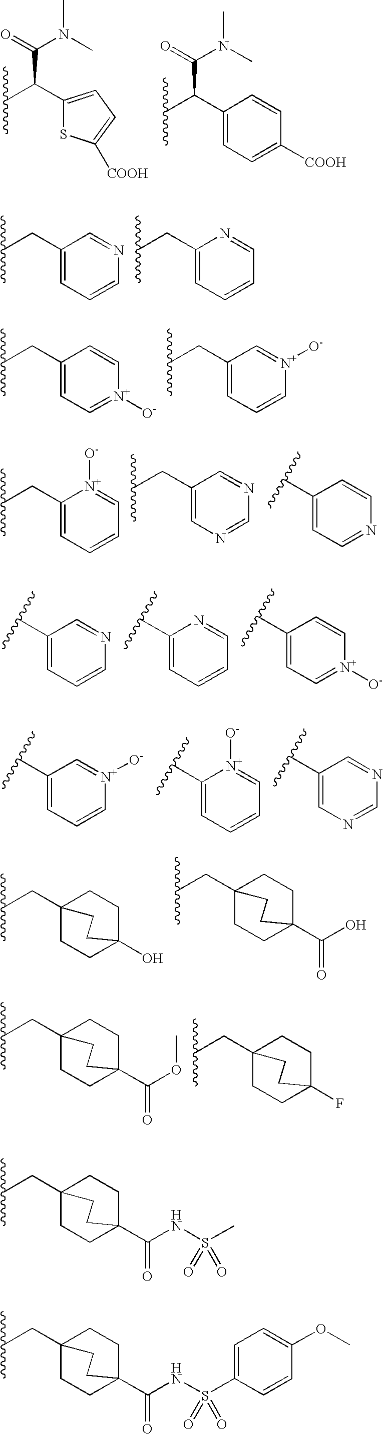

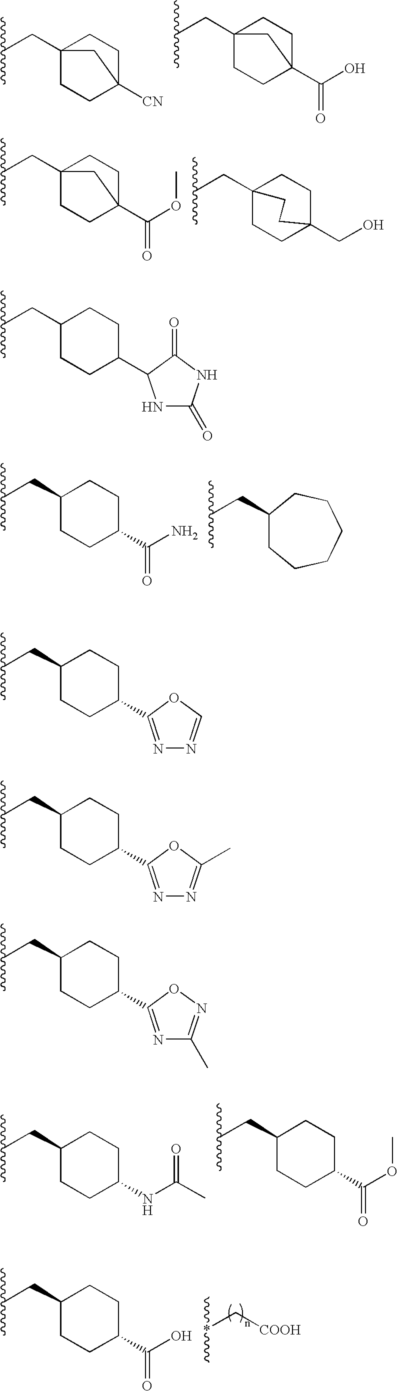

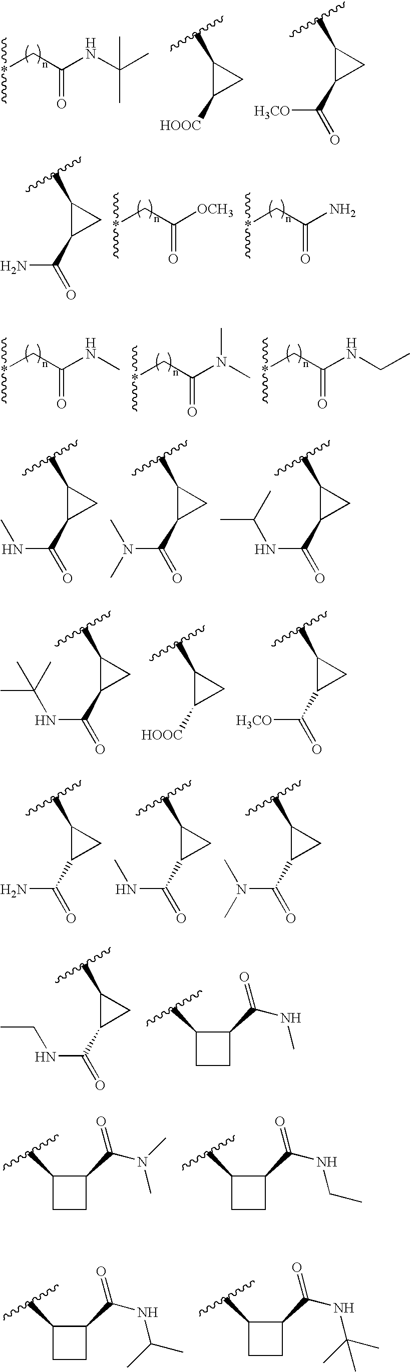

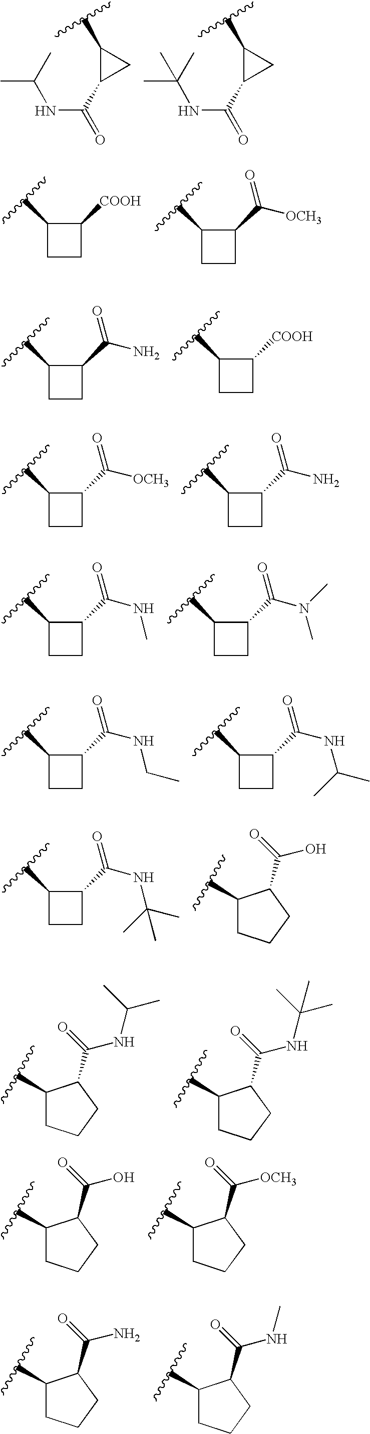

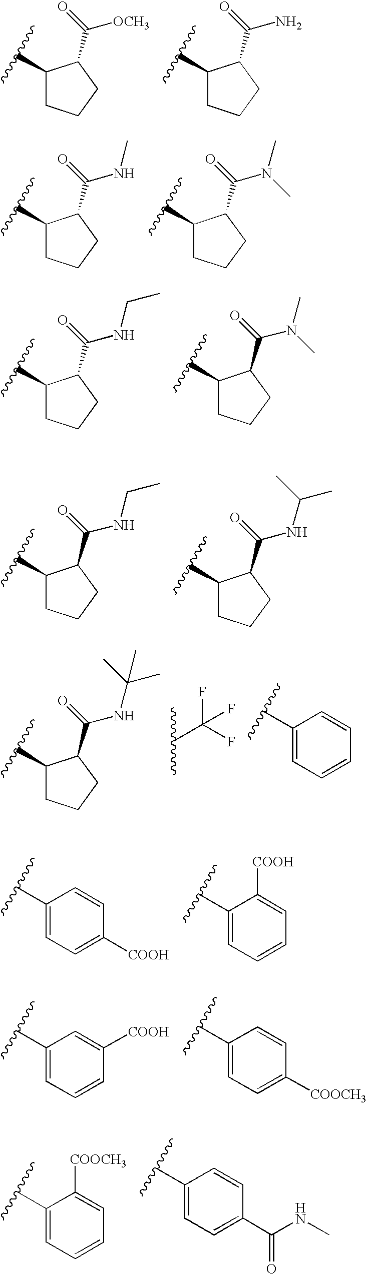

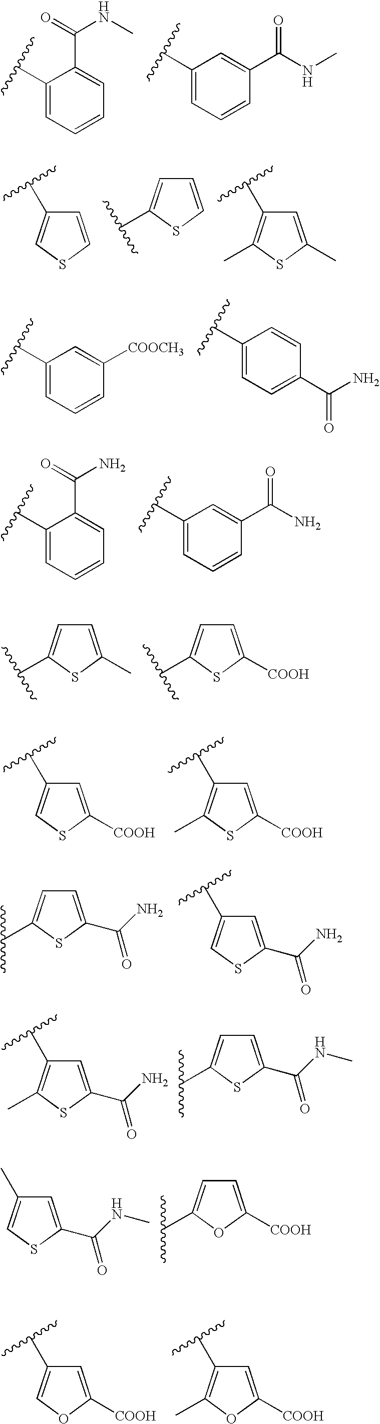

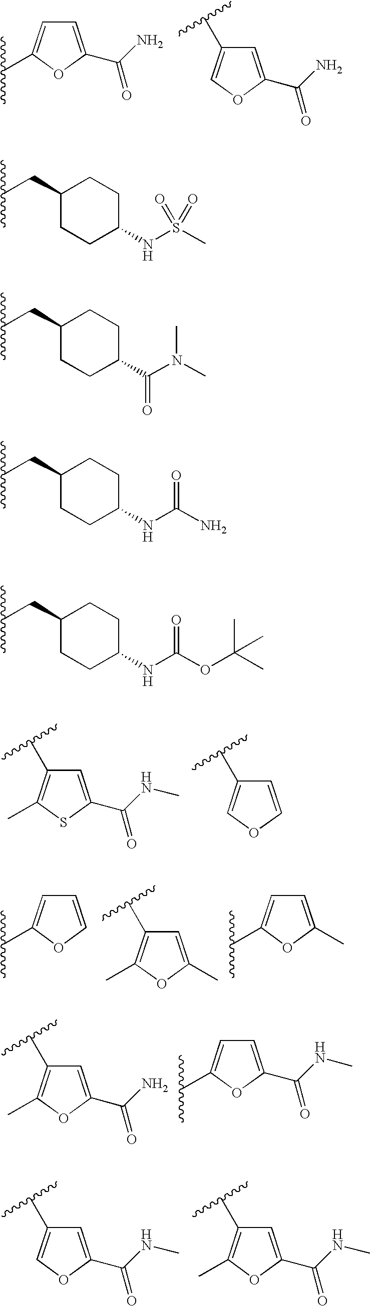

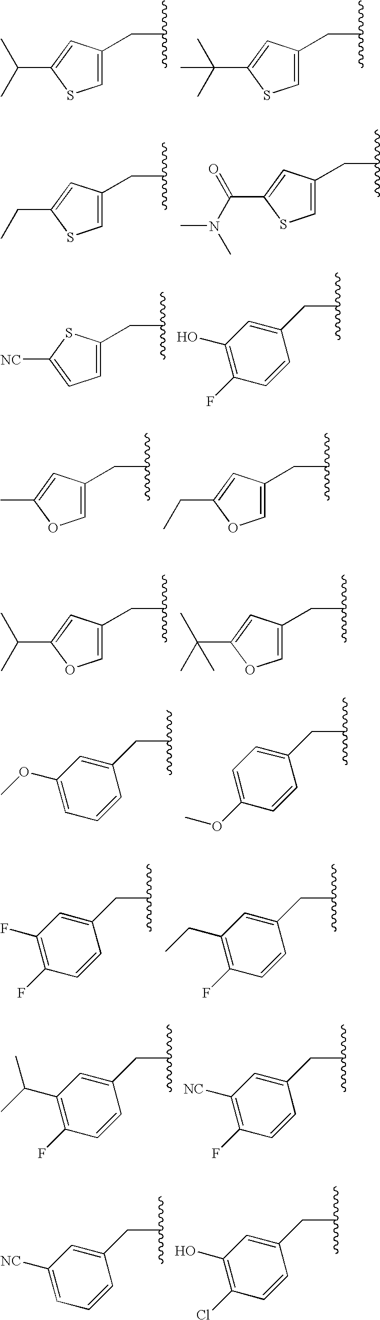

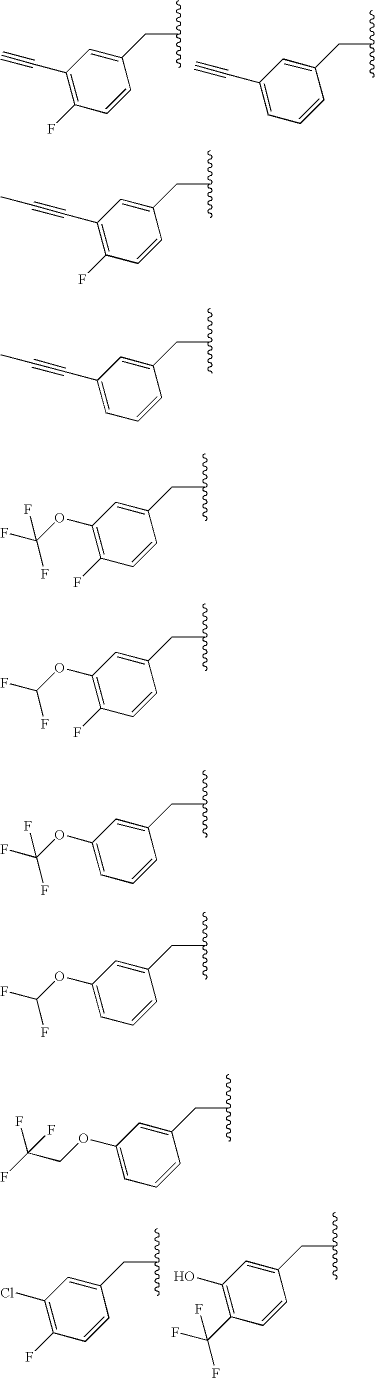

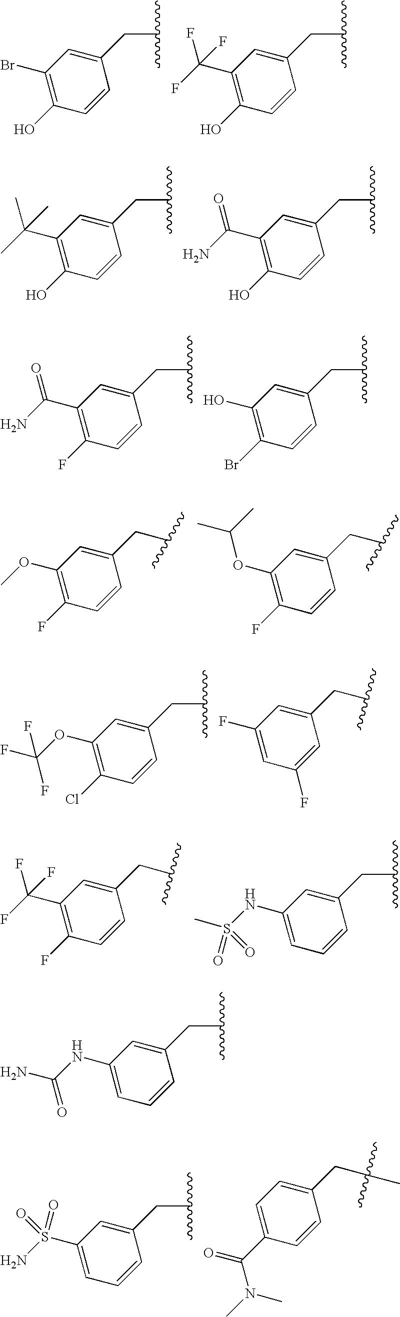

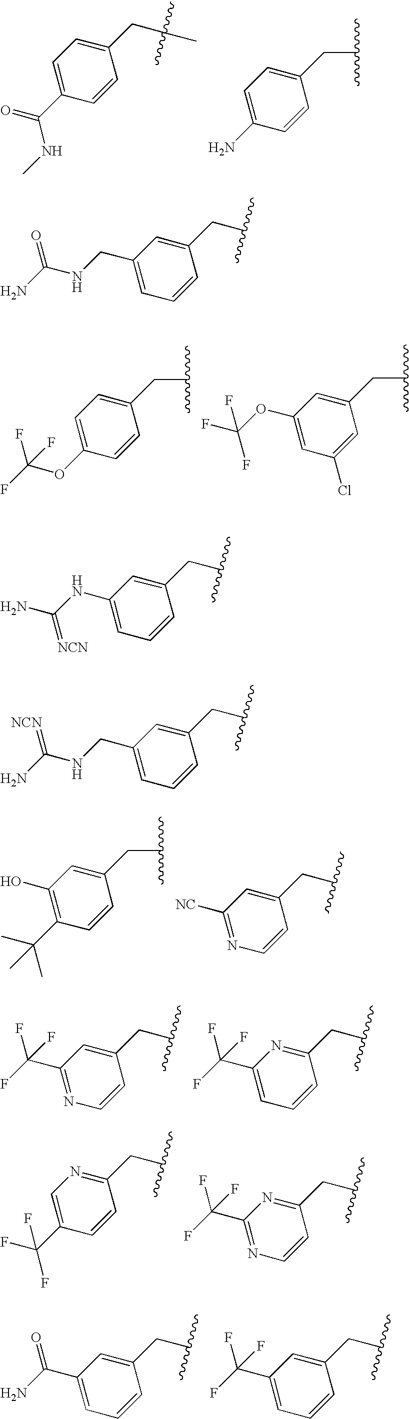

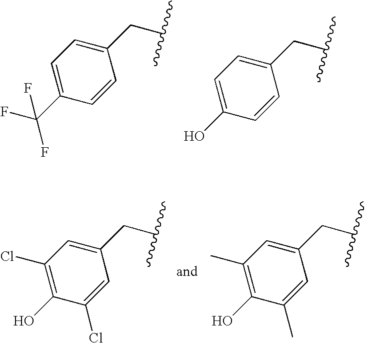

In another embodiment, in conjunction any above or below embodiments, Q_{y}=NR¹R²; and the R¹ of Qy is selected from:

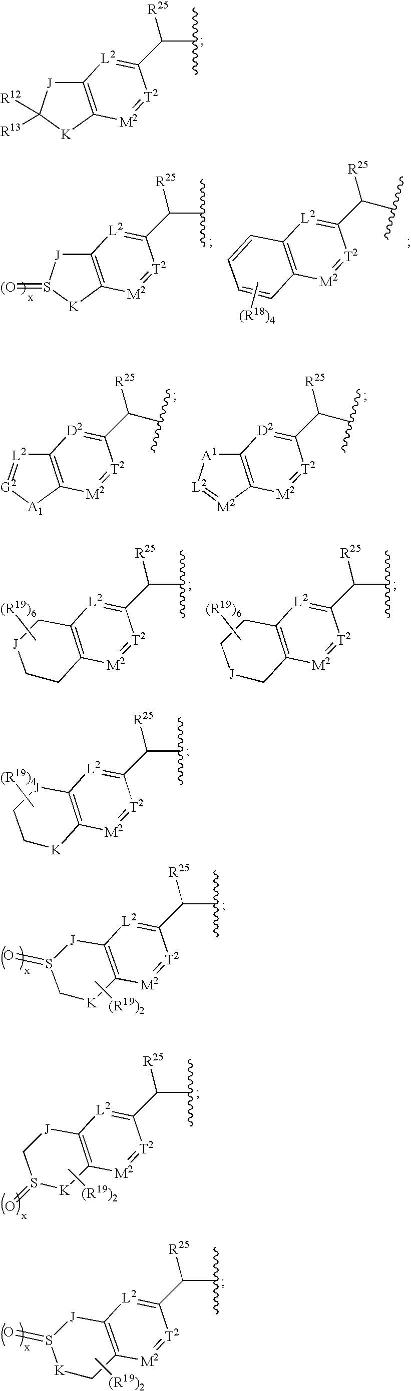

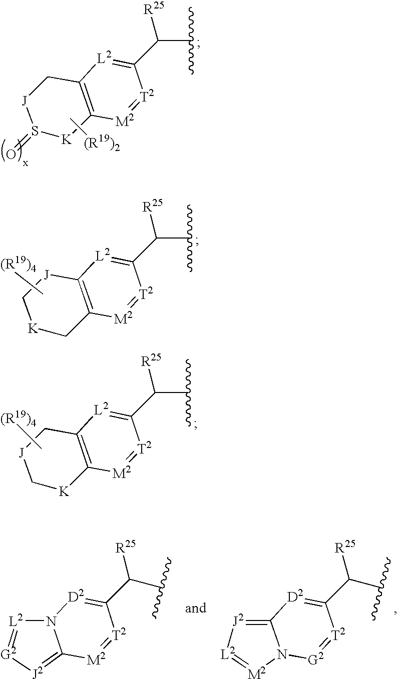

wherein:

R¹² and R¹³ are independently selected from hydrogen, alkyl and halo, wherein alkyl is optionally substituted one or more times, or optionally R¹² and R¹³ together form ═O, ═S or ═NR¹⁰;

R¹⁸ is independently selected from hydrogen, alkyl, haloalkyl, cycloalkyl, heterocycloalkyl, alkynyl, aryl, heteroaryl, —OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹⁰, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times;

R¹⁹ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹⁰, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times, or optionally two R¹⁹ groups together at one carbon atom form ═O, ═S or ═NR¹⁰;

R²⁵ is selected from hydrogen, alkyl, cycloalkyl, C(O)NR¹⁰R¹¹ and haloalkyl, wherein alkyl, cycloalkyl, and haloalkyl are optionally substituted one or more times;

J and K are independently selected from CR¹⁰R¹⁸, NR¹⁰, O and S(O)ₓ;

A₁ is selected from NR¹⁰, O and S;

D², G², J², L², M² and T² are independently selected from CR¹⁸ and N.

In another embodiment, in conjunction any above or below embodiments, Q_{y}=NR¹R²; and the R¹ of Q_{y} is selected from:

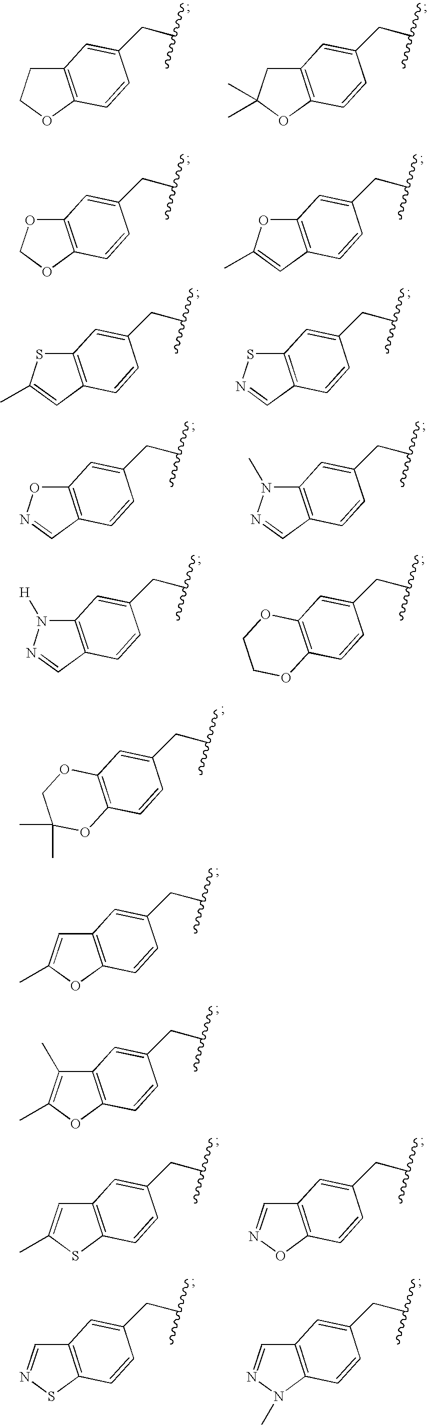

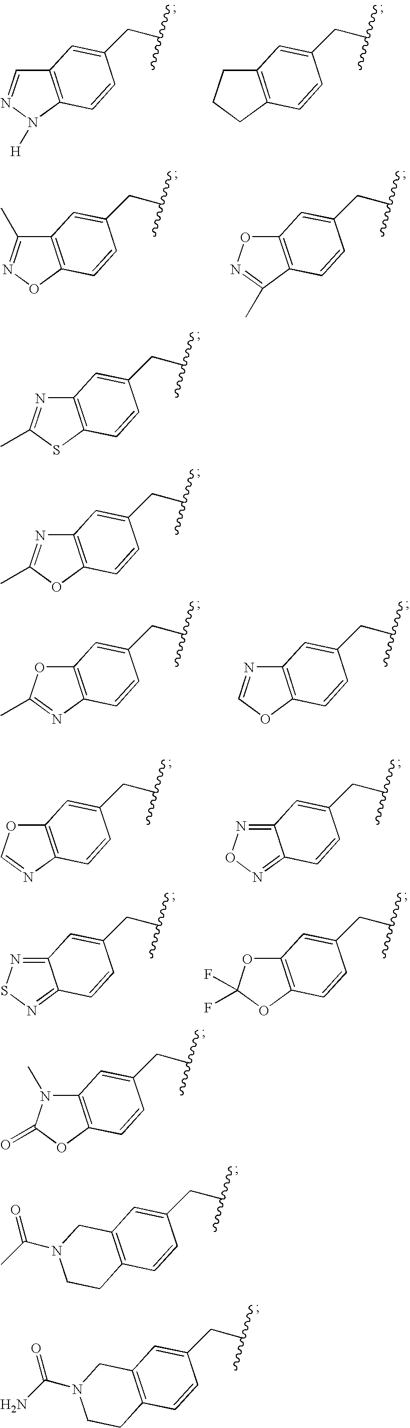

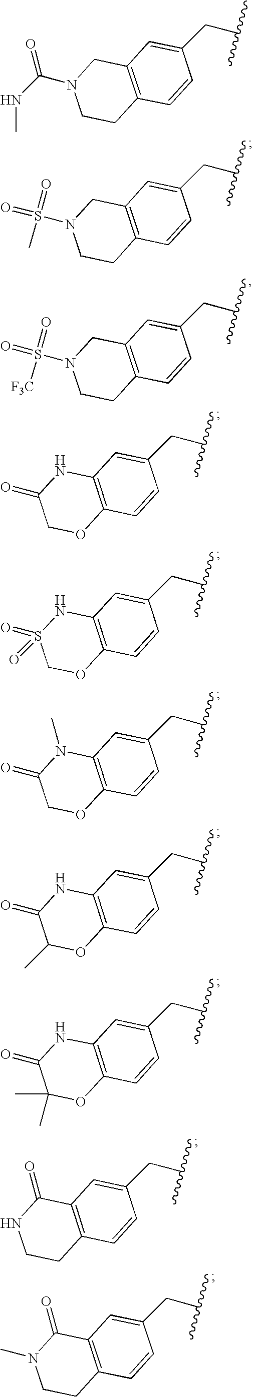

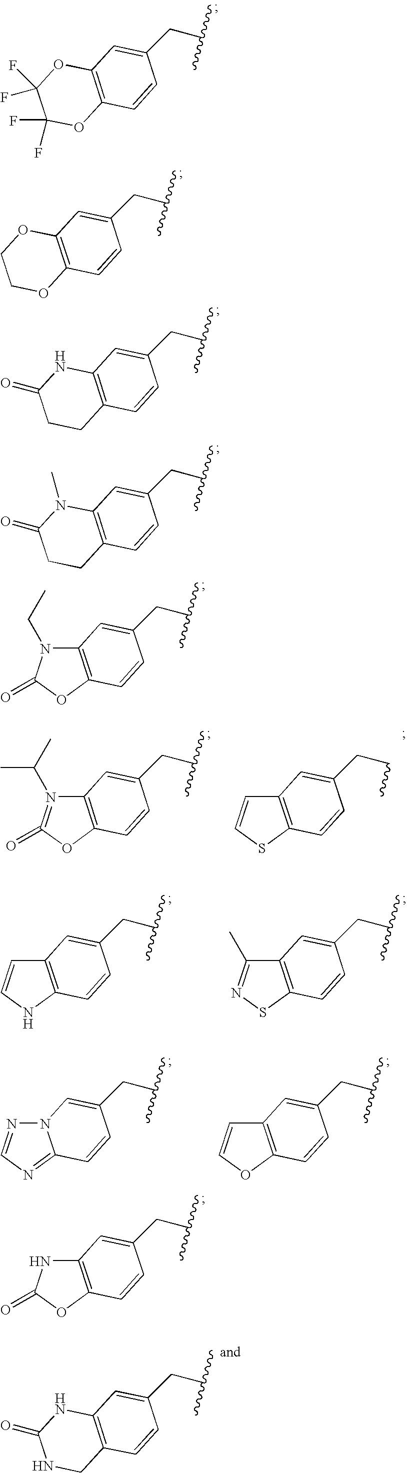

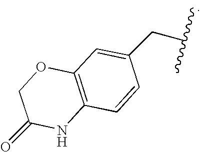

In another embodiment, in conjunction any above or below embodiments, Q_{y}=NR¹R²; and the R¹ of Q_{y} is selected from:

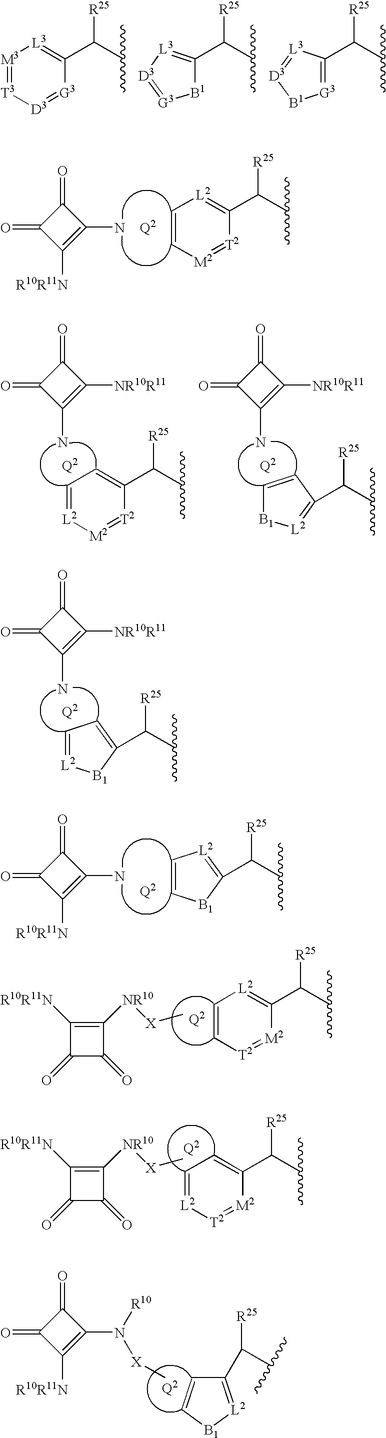

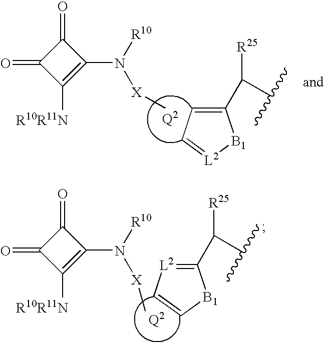

wherein:

R⁵ is independently selected from hydrogen, alkyl, C(O)NR¹⁰R¹¹, aryl, arylalkyl, SO₂NR¹⁰R¹¹ and C(O)OR¹⁰ wherein alkyl, aryl and arylalkyl are optionally substituted one or more times;

R¹⁸ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times;

R¹⁹ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹⁰R¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times, or optionally two R¹⁹ groups together at one carbon atom form ═O, ═S or ═NR¹⁰;

R²⁵ is selected from hydrogen, alkyl, cycloalkyl, CONR¹⁰R¹¹ and haloalkyl, wherein alkyl, cycloalkyl and haloalkyl are optionally substituted one or more times;

L², M², and T² are independently selected from CR¹⁸ and N;

L³, M³, T³, D³, and G³ are independently selected from N, CR¹⁸, (i), or (ii);

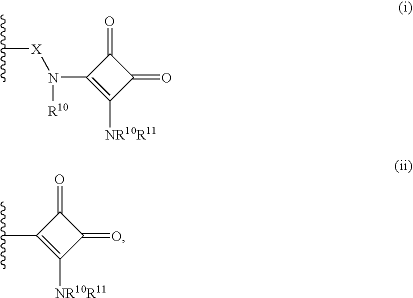

with the provision that one of L³, M³, T³, D³, and G³ is (i) or (ii);

B₁ is selected from the group consisting of NR¹⁰, O and S(O)ₓ;

X is selected from a bond and (CR¹⁰R¹¹)_{w}E(CR¹⁰R¹¹)_{w}

E is selected from a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—, —CH₂—W¹— and

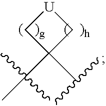

W¹ is selected from O, NR⁵, S, S═O, S(═O)₂, N(R¹⁰)(C═O), N(R¹⁰)S(═O)₂ and S(═O)₂N(R¹⁰);

U is selected from C(R⁵R¹⁰), NR⁵, O, S, S═O, S(═O)₂;

g and h are independently selected from 0-2;

w is selected from 0-4; and

Q² is a 5- to 8-membered ring consisting of cycloalkyl, heterocycloalkyl, aryl, heteroaryl, which is optionally substituted one or more times with R¹⁹.

In another embodiment, in conjunction any above or below embodiments, Q_{y}=NR¹R²; and R¹ is selected from:

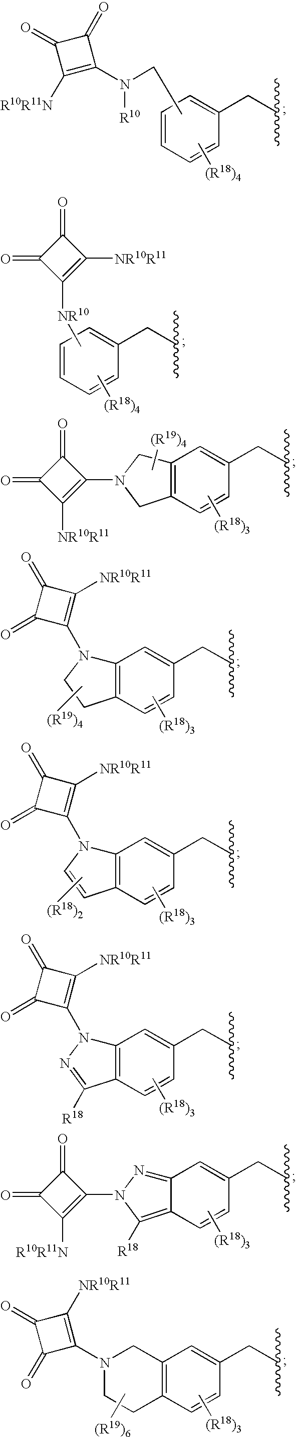

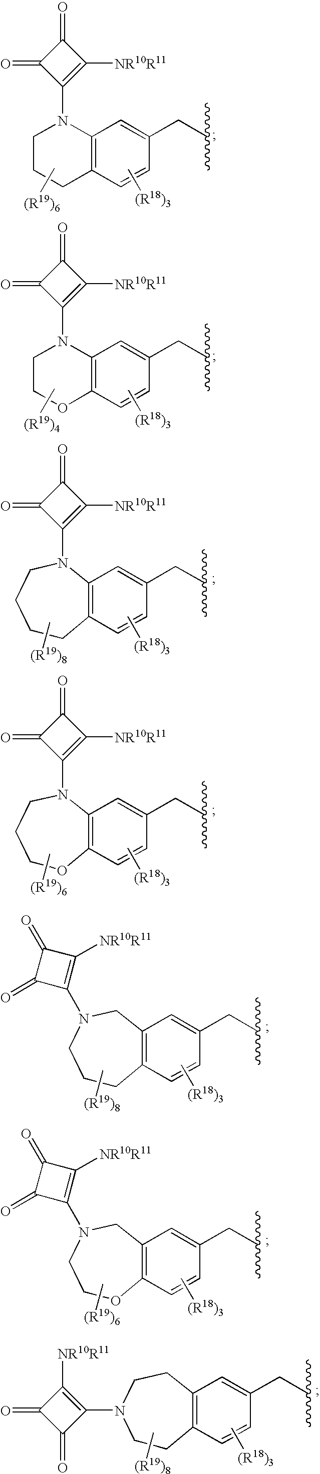

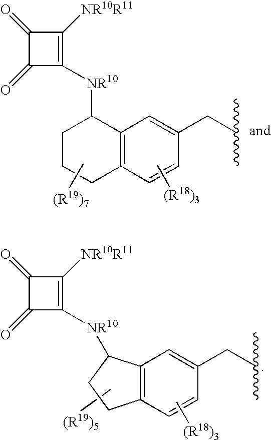

In another embodiment, in conjunction any above or below embodiments, Q_{y}=NR¹R², and the R¹ of Qy is selected from:

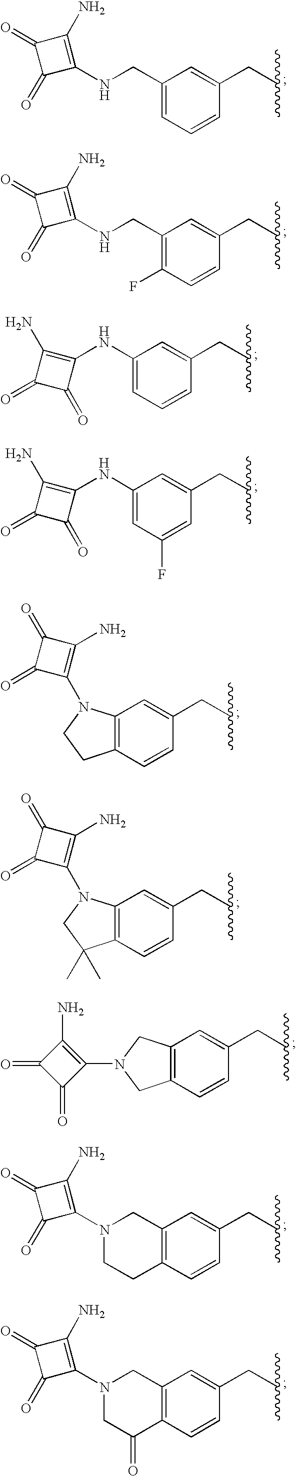

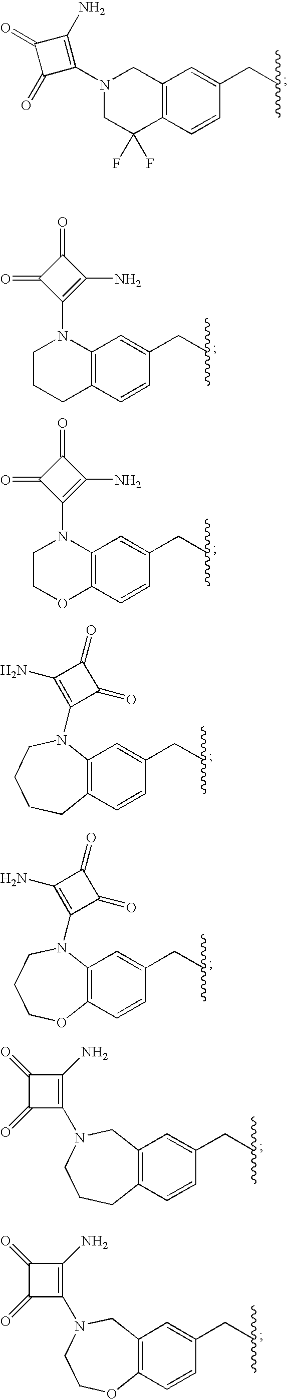

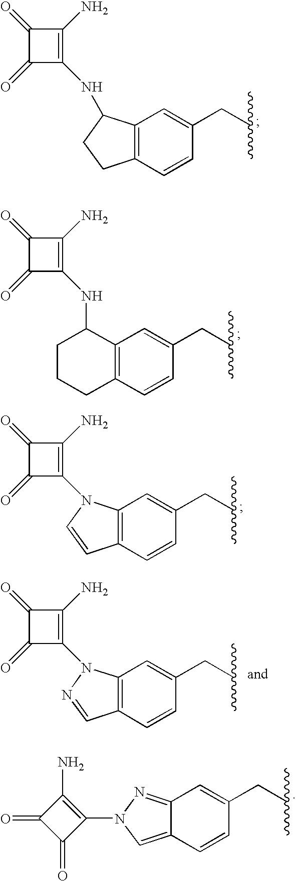

In another embodiment, in conjunction any above or below embodiments, the compound is selected from:

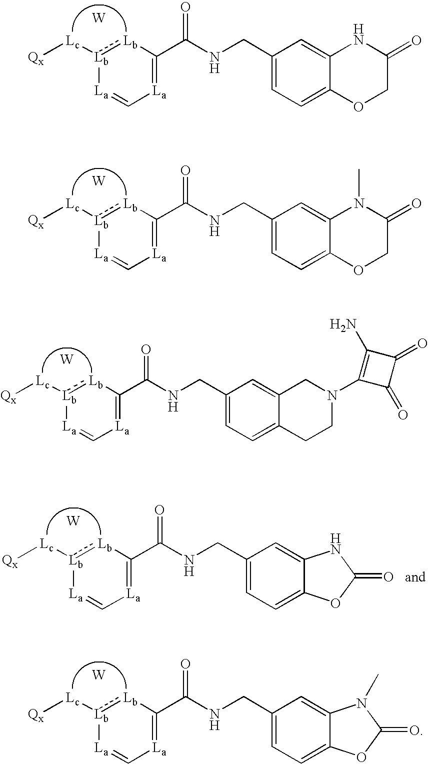

In another embodiment, in conjunction any above or below embodiments, the compound has the structure:

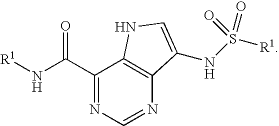

In another embodiment, in conjunction any above or below embodiments, the compound is selected from:

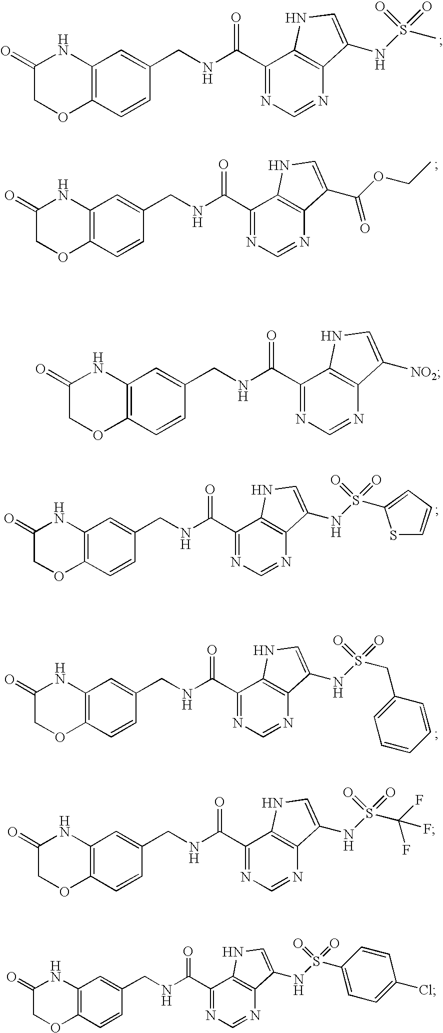

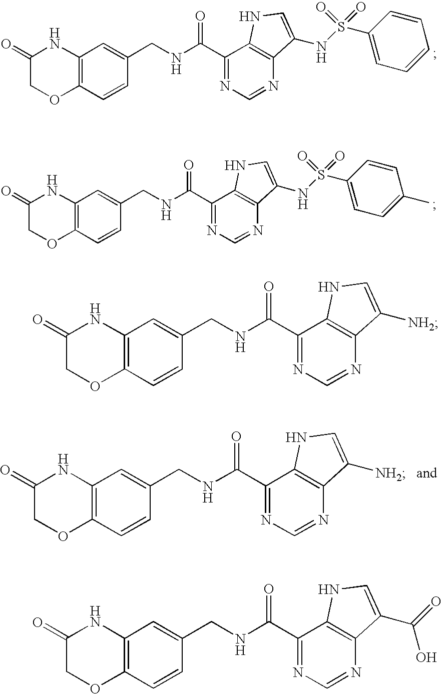

or a pharmaceutically acceptable salt thereof.

In another embodiment, in conjunction with any of the above or below embodiments, the R¹ that is not in Q_{y}, is independently selected from hydrogen, alkyl, haloalkyl, trifluoroalkyl, alkenyl, alkynyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl, any of which are optionally substituted by one R¹⁶ group and optionally substituted by one or more R⁶ groups.

In another embodiment, in conjunction with any of the above or below embodiments, the R¹ that is not in Q_{y} is alkyl, alkenyl, alkynyl or cycloalkyl, any of which are optionally substituted by one R¹⁶ group and optionally substituted by one or more R⁶ groups.

In another embodiment, in conjunction with any of the above or below embodiments, the R¹ that is not in Q_{y}, is heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl, any of which are optionally substituted by one R¹⁶ group and optionally substituted by one or more R⁶ groups.

Another aspect of the invention relates to a pharmaceutical composition comprising an effective amount of the compound according to any of the above or below embodiments.

Another aspect of the invention relates to a method of treating a metalloprotease mediated disease, comprising administering to a subject in need of such treatment an effective amount of a compound according to any of the above or below embodiments.

In another embodiment, in conjunction with any above or below embodiments, the disease is selected from rheumatoid arthritis, osteoarthritis, inflammation, atherosclerosis and multiple sclerosis.

Another aspect of the invention relates to a pharmaceutical composition comprising:

A) an effective amount of a compound according to any of the above or below embodiments;
B) a pharmaceutically acceptable carrier; and
C) a drug, agent or therapeutic selected from: (a) a disease modifying antirheumatic drug; (b) a nonsteroidal anti-inflammatory drug; (c) a COX-2 selective inhibitor; (d) a COX-1 inhibitor; (e) an immunosuppressive; (f) a steroid; (g) a biological response modifier; and (h) a small molecule inhibitor of pro-inflammatory cytokine production.

Another aspect of the invention relates to a method of inhibiting a metalloprotease enzyme, comprising administering a compound according to any of the above or below embodiments.

In another embodiment, in conjunction with any above or below embodiments, the metalloproteinase is selected from MMP-2, MMP-3, MMP-8, and MMP-13.

In another embodiment, in conjunction with any above or below embodiments, the disease is selected from the group consisting of: rheumatoid arthritis, osteoarthritis, abdominal aortic aneurysm, cancer (e.g. but not limited to melanoma, gastric carcinoma or non-small cell lung carcinoma), inflammation, atherosclerosis, chronic obstructive pulmonary disease, ocular diseases (e.g. but not limited to ocular inflammation, retinopathy of prematurity, macular degeneration with the wet type preferred and corneal neovascularization), neurologic diseases, psychiatric diseases, thrombosis, bacterial infection, Parkinson's disease, fatigue, tremor, diabetic retinopathy, vascular diseases of the retina, aging, dementia, cardiomyopathy, renal tubular impairment, diabetes, psychosis, dyskinesia, pigmentary abnormalities, deafness, inflammatory and fibrotic syndromes, intestinal bowel syndrome, allergies, Alzheimers disease, arterial plaque formation, oncology, periodontal, viral infection, stroke, atherosclerosis, cardiovascular disease, reperfusion injury, trauma, chemical exposure or oxidative damage to tissues, wound healing, hemorroid, skin beautifying, pain, inflammatory pain, bone pain and joint pain, acne, acute alcoholic hepatitis, acute inflammation, acute pancreatitis, acute respiratory distress syndrome, adult respiratory disease, airflow obstruction, airway hyperresponsiveness, alcoholic liver disease, allograft rejections, angiogenesis, angiogenic ocular disease, arthritis, asthma, atopic dermatitis, bronchiectasis, bronchiolitis, bronchiolitis obliterans, burn therapy, cardiac and renal reperfusion injury, celiac disease, cerebral and cardiac ischemia, CNS tumors, CNS vasculitis, colds, contusions, cor pulmonae, cough, Crohn's disease, chronic bronchitis, chronic inflammation, chronic pancreatitis, chronic sinusitis, crystal induced arthritis, cystic fibrosis, delayed type hypersensitivity reaction, duodenal ulcers, dyspnea, early transplantation rejection, emphysema, encephalitis, endotoxic shock, esophagitis, gastric ulcers, gingivitis, glomerulonephritis, glossitis, gout, graft vs. host reaction, gram negative sepsis, granulocytic ehrlichiosis, hepatitis viruses, herpes, herpes viruses, HIV, hypercapnea, hyperinflation, hyperoxia-induced inflammation, hypoxia, hypersensitivity, hypoxemia, inflammatory bowel disease, interstitial pneumonitis, ischemia reperfusion injury, kaposi's sarcoma associated virus, lupus, malaria, meningitis, multi-organ dysfunction, necrotizing enterocolitis, osteoporosis, chronic periodontitis, periodontitis, peritonitis associated with continous ambulatory peritoneal dialysis (CAPD), pre-term labor, polymyositis, post surgical trauma, pruritis, psoriasis, psoriatic arthritis, pulmatory fibrosis, pulmatory hypertension, renal reperfusion injury, respiratory viruses, restinosis, right ventricular hypertrophy, sarcoidosis, septic shock, small airway disease, sprains, strains, subarachnoid hemorrhage, surgical lung volume reduction, thrombosis, toxic shock syndrome, transplant reperfusion injury, traumatic brain injury, ulcerative colitis, vasculitis, ventilation-perfusion mismatching, and wheeze.

Another aspect of the invention relates to the use of a compound according to any of the above or below embodiments for the manufacture of a medicament for treating an metalloprotease mediated disease.

In another embodiment, in conjunction with any of the above or below embodiments, the metalloprotease mediated disease is selected from the group consisting of MMP-2, MMP-3, MMP-8 and MMP-13 mediated diseases.

The specification and claims contain listing of species using the language “selected from . . . and . . . ” and “is . . . or . . . ” (sometimes referred to as Markush groups). When this language is used in this application, unless otherwise stated it is meant to include the group as a whole, or any single members thereof, or any subgroups thereof. The use of this language is merely for shorthand purposes and is not meant in any way to limit the removal of individual elements or subgroups as needed.

The terms “alkyl” or “alk”, as used herein alone or as part of another group, denote optionally substituted, straight and branched chain saturated hydrocarbon groups, preferably having 1 to 10 carbons in the normal chain, most preferably lower alkyl groups. Exemplary unsubstituted such groups include methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl, dodecyl and the like. Exemplary substituents may include, but are not limited to, one or more of the following groups: halo, alkoxy, alkylthio, alkenyl, alkynyl, aryl (e.g., to form a benzyl group), cycloalkyl, cycloalkenyl, hydroxy or protected hydroxy, carboxyl (—COOH), alkyloxycarbonyl, alkylcarbonyloxy, alkylcarbonyl, carbamoyl (NH₂—CO—), substituted carbamoyl ((R¹⁰)(R¹¹)N—CO— wherein R¹⁰ or R¹¹ are as defined below, except that at least one of R¹⁰ or R¹¹ is not hydrogen), amino, heterocyclo, mono- or dialkylamino, or thiol (—SH).

The terms “lower alk” or “lower alkyl” as used herein, denote such optionally substituted groups as described above for alkyl having 1 to 4 carbon atoms in the normal chain.

The term “alkoxy” denotes an alkyl group as described above bonded through an oxygen linkage (—O—).

The term “alkenyl”, as used herein alone or as part of another group, denotes optionally substituted, straight and branched chain hydrocarbon groups containing at least one carbon to carbon double bond in the chain, and preferably having 2 to 10 carbons in the normal chain. Exemplary unsubstituted such groups include ethenyl, propenyl, isobutenyl, butenyl, pentenyl, hexenyl, heptenyl, octenyl, nonenyl, decenyl, and the like. Exemplary substituents may include, but are not limited to, one or more of the following groups: halo, alkoxy, alkylthio, alkyl, alkynyl, aryl, cycloalkyl, cycloalkenyl, hydroxy or protected hydroxy, carboxyl (—COOH), alkyloxycarbonyl, alkylcarbonyloxy, alkylcarbonyl, carbamoyl (NH₂—CO—), substituted carbamoyl ((R¹⁰)(R¹¹)N—CO— wherein R¹⁰ or R¹¹ are as defined below, except that at least one of R¹⁰ or R¹¹ is not hydrogen), amino, heterocyclo, mono- or dialkylamino, or thiol (—SH).

The term “alkynyl”, as used herein alone or as part of another group, denotes optionally substituted, straight and branched chain hydrocarbon groups containing at least one carbon to carbon triple bond in the chain, and preferably having 2 to 10 carbons in the normal chain. Exemplary unsubstituted such groups include, but are not limited to, ethynyl, propynyl, butynyl, pentynyl, hexynyl, heptynyl, octynyl, nonynyl, decynyl, and the like. Exemplary substituents may include, but are not limited to, one or more of the following groups: halo, alkoxy, alkylthio, alkyl, alkenyl, aryl, cycloalkyl, cycloalkenyl, hydroxy or protected hydroxy, carboxyl (—COOH), alkyloxycarbonyl, alkylcarbonyloxy, alkylcarbonyl, carbamoyl (NH₂—CO—), substituted carbamoyl ((R¹⁰)(R¹¹)N—CO— wherein R¹⁰ or R¹¹ are as defined below, except that at least one of R¹⁰ or R¹¹ is not hydrogen), amino, heterocyclo, mono- or dialkylamino, or thiol (—SH).

The term “cycloalkyl”, as used herein alone or as part of another group, denotes optionally substituted, saturated cyclic hydrocarbon ring systems, containing one ring with 3 to 9 carbons. Exemplary unsubstituted such groups include, but are not limited to, cyclopropyl, cyclobutyl, cyclopentyl, cyclohexyl, cycloheptyl, cyclooctyl, cyclodecyl, and cyclododecyl. Exemplary substituents include, but are not limited to, one or more alkyl groups as described above, or one or more groups described above as alkyl substituents.

The term “bicycloalkyl”, as used herein alone or as part of another group, denotes optionally substituted, saturated cyclic bridged hydrocarbon ring systems, desirably containing 2 or 3 rings and 3 to 9 carbons per ring. Exemplary unsubstituted such groups include, but are not limited to, adamantyl, bicyclo[2.2.2]octane, bicyclo[2.2.1]heptane and cubane. Exemplary substituents include, but are not limited to, one or more alkyl groups as described above, or one or more groups described above as alkyl substituents.

The term “spiroalkyl”, as used herein alone or as part of another group, denotes an optionally substituted, saturated hydrocarbon ring systems, wherein two rings of 3 to 9 carbons per ring are bridged via one carbon atom. Exemplary unsubstituted such groups include, but are not limited to, spiro[3.5]nonane, spiro[4.5]decane or spiro[2.5]octane. Exemplary substituents include, but are not limited to, one or more alkyl groups as described above, or one or more groups described above as alkyl substituents.

The term “spiroheteroalkyl”, as used herein alone or as part of another group, denotes an optionally substituted, saturated hydrocarbon ring systems, wherein two rings of 3 to 9 carbons per ring are bridged via one carbon atom. At least one carbon atom is replaced by a heteroatom independently selected from N, O, and S. The nitrogen and sulfur heteroatoms may optionally be oxidized. Exemplary unsubstituted such groups include, but are not limited to, 1,3-diaza-spiro[4.5]decane-2,4-dione. Exemplary substituents include, but are not limited to, one or more alkyl groups as described above, or one or more groups described above as alkyl substituents.

The terms “ar” or “aryl”, as used herein alone or as part of another group, denote optionally substituted, homocyclic aromatic groups, preferably containing 1 or 2 rings and 6 to 12 ring carbons. Exemplary unsubstituted such groups include, but are not limited to, phenyl, biphenyl, and naphthyl. Exemplary substituents include, but are not limited to, one or more nitro groups, alkyl groups as described above or groups described above as alkyl substituents.

The term “heterocycle” or “heterocyclic system” denotes a heterocyclyl, heterocyclenyl, or heteroaryl group as described herein, which contains carbon atoms and from 1 to 4 heteroatoms independently selected from N, O and S and including any bicyclic or tricyclic group in which any of the above-defined heterocyclic rings is fused to one or more heterocycle, aryl or cycloalkyl groups. The nitrogen and sulfur heteroatoms may optionally be oxidized. The heterocyclic ring may be attached to its pendant group at any heteroatom or carbon atom which results in a stable structure. The heterocyclic rings described herein may be substituted on carbon or on a nitrogen atom.

Examples of heterocycles include, but are not limited to, 1H-indazole, 2-pyrrolidonyl, 2H,6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azocinyl, benzimidazolyl, benzofuranyl, benzothiofuranyl, benzothiophenyl, benzoxazolinyl, benzoxazolyl, benzthiazolyl, benztriazolyl, benztetrazolyl, benzisoxazolyl, benzisothiazolyl, benzimidazalonyl, carbazolyl, 4aH-carbazolyl, b-carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, isatinoyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinylperimidinyl, oxindolyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, 4H-quinolizinyl, quinoxalinyl, quinuclidinyl, carbolinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, xanthenyl.

Further examples of heterocycles include, but not are not limited to, “heterobicycloalkyl” groups such as 7-oxa-bicyclo[2.2.1]heptane, 7-aza-bicyclo[2.2.1]heptane, and 1-aza-bicyclo[2.2.2]octane.

“Heterocyclenyl” denotes a non-aromatic monocyclic or multicyclic hydrocarbon ring system of about 3 to about 10 atoms, desirably about 4 to about 8 atoms, in which one or more of the carbon atoms in the ring system is/are hetero element(s) other than carbon, for example nitrogen, oxygen or sulfur atoms, and which contains at least one carbon-carbon double bond or carbon-nitrogen double bond. Ring sizes of rings of the ring system may include 5 to 6 ring atoms. The designation of the aza, oxa or thia as a prefix before heterocyclenyl define that at least a nitrogen, oxygen or sulfur atom is present respectively as a ring atom. The heterocyclenyl may be optionally substituted by one or more substituents as defined herein. The nitrogen or sulphur atom of the heterocyclenyl may also be optionally oxidized to the corresponding N-oxide, S-oxide or S,S-dioxide. “Heterocyclenyl” as used herein includes by way of example and not limitation those described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and “J. Am. Chem. Soc.”, 82:5566 (1960), the contents all of which are incorporated by reference herein. Exemplary monocyclic azaheterocyclenyl groups include, but are not limited to, 1,2,3,4-tetrahydrohydropyridine, 1,2-dihydropyridyl, 1,4-dihydropyridyl, 1,2,3,6-tetrahydropyridine, 1,4,5,6-tetrahydropyrimidine, 2-pyrrolinyl, 3-pyrrolinyl, 2-imidazolinyl, 2-pyrazolinyl, and the like. Exemplary oxaheterocyclenyl groups include, but are not limited to, 3,4-dihydro-2H-pyran, dihydrofuranyl, and fluorodihydrofuranyl. An exemplary multicyclic oxaheterocyclenyl group is 7-oxabicyclo[2.2.1]heptenyl.

“Heterocyclyl,” or “heterocycloalkyl,” denotes a non-aromatic saturated monocyclic or multicyclic ring system of about 3 to about 10 carbon atoms, desirably 4 to 8 carbon atoms, in which one or more of the carbon atoms in the ring system is/are hetero element(s) other than carbon, for example nitrogen, oxygen or sulfur. Ring sizes of rings of the ring system may include 5 to 6 ring atoms. The designation of the aza, oxa or thia as a prefix before heterocyclyl define that at least a nitrogen, oxygen or sulfur atom is present respectively as a ring atom. The heterocyclyl may be optionally substituted by one or more substituents which may be the same or different, and are as defined herein. The nitrogen or sulphur atom of the heterocyclyl may also be optionally oxidized to the corresponding N-oxide, S-oxide or S,S-dioxide.

“Heterocyclyl” as used herein includes by way of example and not limitation those described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and “J. Am. Chem. Soc.”, 82:5566 (1960). Exemplary monocyclic heterocyclyl rings include, but are not limited to, piperidyl, pyrrolidinyl, piperazinyl, morpholinyl, thiomorpholinyl, thiazolidinyl, 1,3-dioxolanyl, 1,4-dioxanyl, tetrahydrofuranyl, tetrahydrothiophenyl, tetrahydrothiopyranyl, and the like.

“Heteroaryl” denotes an aromatic monocyclic or multicyclic ring system of about 5 to about 10 atoms, in which one or more of the atoms in the ring system is/are hetero element(s) other than carbon, for example nitrogen, oxygen or sulfur. Ring sizes of rings of the ring system include 5 to 6 ring atoms. The “heteroaryl” may also be substituted by one or more substituents which may be the same or different, and are as defined herein. The designation of the aza, oxa or thia as a prefix before heteroaryl define that at least a nitrogen, oxygen or sulfur atom is present respectively as a ring atom. A nitrogen atom of a heteroaryl may be optionally oxidized to the corresponding N-oxide. Heteroaryl as used herein includes by way of example and not limitation those described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and “J. Am. Chem. Soc.”, 82:5566 (1960). Exemplary heteroaryl and substituted heteroaryl groups include, but are not limited to, pyrazinyl, thienyl, isothiazolyl, oxazolyl, pyrazolyl, furazanyl, pyrrolyl, 1,2,4-thiadiazolyl, pyridazinyl, quinoxalinyl, phthalazinyl, imidazo[1,2-a]pyridine, imidazo[2,1-b]thiazolyl, benzofurazanyl, azaindolyl, benzimidazolyl, benzothienyl, thienopyridyl, thienopyrimidyl, pyrrolopyridyl, imidazopyridyl, benzoazaindole, 1,2,3-triazinyl, 1,2,4-triazinyl, 1,3,5-triazinyl, benzthiazolyl, dioxolyl, furanyl, imidazolyl, indolyl, indolizinyl, isoxazolyl, isoquinolinyl, isothiazolyl, oxadiazolyl, oxazinyl, oxiranyl, piperazinyl, piperidinyl, pyranyl, pyrazinyl, pyridazinyl, pyrazolyl, pyridyl, pyrimidinyl, pyrrolyl, pyrrolidinyl, quinazolinyl, quinolinyl, tetrazinyl, tetrazolyl, 1,3,4-thiadiazolyl, 1,2,3-thiadiazolyl, 1,2,4-thiadiazolyl, 1,2,5-thiadiazolyl, thiatriazolyl, thiazinyl, thiazolyl, thienyl, 5-thioxo-1,2,4-diazolyl, thiomorpholino, thiophenyl, thiopyranyl, triazolyl and triazolonyl.

The phrase “fused” means, that the group, mentioned before “fused” is connected via two adjacent atoms to the ring system mentioned after “fused” to form a bicyclic system. For example, “heterocycloalkyl fused aryl” includes, but is not limited to, 2,3-dihydro-benzo[1,4]dioxine, 4H-benzo[1,4]oxazin-3-one, 3H-Benzooxazol-2-one and 3,4-dihydro-2H-benzo[f][1,4]oxazepin-5-one.

The term “amino” denotes the radical —NH₂ wherein one or both of the hydrogen atoms may be replaced by an optionally substituted hydrocarbon group. Exemplary amino groups include, but are not limited to, n-butylamino, tert-butylamino, methylpropylamino and ethyldimethylamino.

The term “cycloalkylalkyl” denotes a cycloalkyl-alkyl group wherein a cycloalkyl as described above is bonded through an alkyl, as defined above. Cycloalkylalkyl groups may contain a lower alkyl moiety. Exemplary cycloalkylalkyl groups include, but are not limited to, cyclopropylmethyl, cyclopentylmethyl, cyclohexylmethyl, cyclopropylethyl, cyclopentylethyl, cyclohexylpropyl, cyclopropylpropyl, cyclopentylpropyl, and cyclohexylpropyl.

The term “arylalkyl” denotes an aryl group as described above bonded through an alkyl, as defined above.

The term “heteroarylalkyl” denotes a heteroaryl group as described above bonded through an alkyl, as defined above.

The term “heterocyclylalkyl,” or “heterocycloalkylalkyl,” denotes a heterocyclyl group as described above bonded through an alkyl, as defined above.

The terms “halogen”, “halo”, or “hal”, as used herein alone or as part of another group, denote chlorine, bromine, fluorine, and iodine.

The term “haloalkyl” denotes a halo group as described above bonded though an alkyl, as defined above. Fluoroalkyl is an exemplary group.

The term “aminoalkyl” denotes an amino group as defined above bonded through an alkyl, as defined above.

The phrase “bicyclic fused ring system wherein at least one ring is partially saturated” denotes an 8- to 13-membered fused bicyclic ring group in which at least one of the rings is non-aromatic. The ring group has carbon atoms and optionally 1-4 heteroatoms independently selected from N, O and S. Illustrative examples include, but are not limited to, indanyl, tetrahydronaphthyl, tetrahydroquinolyl and benzocycloheptyl.

The phrase “tricyclic fused ring system wherein at least one ring is partially saturated” denotes a 9- to 18-membered fused tricyclic ring group in which at least one of the rings is non-aromatic. The ring group has carbon atoms and optionally 1-7 heteroatoms independently selected from N, O and S. Illustrative examples include, but are not limited to, fluorene, 10,11-dihydro-5H-dibenzo[a,d]cycloheptene and 2,2a,7,7a-tetrahydro-1H-cyclobuta[a]indene.

The term “pharmaceutically acceptable salts” refers to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. Examples therefore may be, but are not limited to, sodium, potassium, choline, lysine, arginine or N-methyl-glucamine salts, and the like.

The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as, but not limited to, hydrochloric, hydrobromic, sulfuric, sulfamic, phosphoric, nitric and the like; and the salts prepared from organic acids such as, but not limited to, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Organic solvents include, but are not limited to, nonaqueous media like ethers, ethyl acetate, ethanol, isopropanol, or acetonitrile. Lists of suitable salts are found in Remington's Pharmaceutical Sciences, 18th ed., Mack Publishing Company, Easton, Pa., 1990, p. 1445, the disclosure of which is hereby incorporated by reference.

The phrase “pharmaceutically acceptable” denotes those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

The phrase “pharmaceutically acceptable carrier” denotes media generally accepted in the art for the delivery of biologically active agents to mammals, e.g., humans. Such carriers are generally formulated according to a number of factors well within the purview of those of ordinary skill in the art to determine and account for. These include, without limitation: the type and nature of the active agent being formulated; the subject to which the agent-containing composition is to be administered; the intended route of administration of the composition; and, the therapeutic indication being targeted. Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, well known to those of ordinary skill in the art. Non-limiting examples of a pharmaceutically acceptable carrier are hyaluronic acid and salts thereof, and microspheres (including, but not limited to poly(D,L)-lactide-co-glycolic acid copolymer (PLGA), poly(L-lactic acid) (PLA), poly(caprolactone (PCL) and bovine serum albumin (BSA)). Descriptions of suitable pharmaceutically acceptable carriers, and factors involved in their selection, are found in a variety of readily available sources, e.g., Remington's Pharmaceutical Sciences, 17th ed., Mack Publishing Company, Easton, Pa., 1985, the contents of which are incorporated herein by reference.

Pharmaceutically acceptable carriers particularly suitable for use in conjunction with tablets include, for example, inert diluents, such as celluloses, calcium or sodium carbonate, lactose, calcium or sodium phosphate; disintegrating agents, such as croscarmellose sodium, cross-linked povidone, maize starch, or alginic acid; binding agents, such as povidone, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example celluloses, lactose, calcium phosphate or kaolin, or as soft gelatin capsules wherein the active ingredient is mixed with non-aqueous or oil medium, such as glycerin, propylene glycol, polyethylene glycol, peanut oil, liquid paraffin or olive oil.

The compositions of the invention may also be formulated as suspensions including a compound of the present invention in admixture with at least one pharmaceutically acceptable excipient suitable for the manufacture of a suspension. In yet another embodiment, pharmaceutical compositions of the invention may be formulated as dispersible powders and granules suitable for preparation of a suspension by the addition of suitable excipients.

Carriers suitable for use in connection with suspensions include suspending agents, such as sodium carboxymethylcellulose, methylcellulose, hydroxypropyl methylcelluose, sodium alginate, polyvinylpyrrolidone, gum tragacanth, gum acacia, dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycethanol), a condensation product of ethylene oxide with a partial ester derived from a fatty acid and a hexitol anhydride (e.g., polyoxyethylene sorbitan monooleate); and thickening agents, such as carbomer, beeswax, hard paraffin or cetyl alcohol. The suspensions may also contain one or more preservatives such as acetic acid, methyl and/or n-propyl p-hydroxy-benzoate; one or more coloring agents; one or more flavoring agents; and one or more sweetening agents such as sucrose or saccharin.

Cyclodextrins may be added as aqueous solubility enhancers. Preferred cyclodextrins include hydroxypropyl, hydroxyethyl, glucosyl, maltosyl and maltotriosyl derivatives of α-, β-, and γ-cyclodextrin. The amount of solubility enhancer employed will depend on the amount of the compound of the present invention in the composition.

The term “formulation” denotes a product comprising the active ingredient(s) and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical formulations of the present invention encompass any composition made by admixing a compound of the present invention and a pharmaceutical carrier.

The term “N-oxide” denotes compounds that can be obtained in a known manner by reacting a compound of the present invention including a nitrogen atom (such as in a pyridyl group) with hydrogen peroxide or a peracid, such as 3-chloroperoxy-benzoic acid, in an inert solvent, such as dichloromethane, at a temperature between about −10-80° C., desirably about 0° C.

The term “polymorph” denotes a form of a chemical compound in a particular crystalline arrangement. Certain polymorphs may exhibit enhanced thermodynamic stability and may be more suitable than other polymorphic forms for inclusion in pharmaceutical formulations.

The compounds of the invention can contain one or more chiral centers and/or double bonds and, therefore, exist as stereoisomers, such as double-bond isomers (i.e., geometric isomers), enantiomers, or diastereomers. According to the invention, the chemical structures depicted herein, and therefore the compounds of the invention, encompass all of the corresponding enantiomers and stereoisomers, that is, both the stereomerically pure form (e.g., geometrically pure, enantiomerically pure, or diastereomerically pure) and enantiomeric and stereoisomeric mixtures.

The term “racemic mixture” denotes a mixture that is about 50% of one enantiomer and about 50% of the corresponding enantiomer relative to all chiral centers in the molecule. Thus, the invention encompasses all enantiomerically-pure, enantiomerically-enriched, and racemic mixtures of compounds of Formula (I).

Enantiomeric and stereoisomeric mixtures of compounds of the invention can be resolved into their component enantiomers or stereoisomers by well-known methods. Examples include, but are not limited to, the formation of chiral salts and the use of chiral or high performance liquid chromatography “HPLC” and the formation and crystallization of chiral salts. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); Wilen, S. H., Tables of Resolving Agents and Optical Resolutions p. 268 (E. L. Eliel, Ed., Univ. of Notre Dame Press, Notre Dame, Ind., 1972); Stereochemistry of Organic Compounds, Ernest L. Eliel, Samuel H. Wilen and Lewis N. Manda (1994 John Wiley & Sons, Inc.), and Stereoselective Synthesis A Practical Approach, Mihaly Nogradi (1995 VCH Publishers, Inc., NY, N.Y.). Enantiomers and stereoisomers can also be obtained from stereomerically- or enantiomerically-pure intermediates, reagents, and catalysts by well-known asymmetric synthetic methods.

“Substituted” is intended to indicate that one or more hydrogens on the atom indicated in the expression using “substituted” is replaced with a selection from the indicated group(s), provided that the indicated atom's normal valency is not exceeded, and that the substitution results in a stable compound. When a substituent is keto (i.e., ═O) group, then 2 hydrogens on the atom are replaced.

Unless moieties of a compound of the present invention are defined as being unsubstituted, the moieties of the compound may be substituted. In addition to any substituents provided above, the moieties of the compounds of the present invention may be optionally substituted with one or more groups independently selected from:

C₁-C₄ alkyl;

C₂-C₄ alkenyl;

C₂-C₄ alkynyl;

CF₃;

halo;

OH;

O—(C₁-C₄ alkyl);

OCH₂F;

OCHF₂;

OCF₃;

ONO₂;

OC(O)—(C₁-C₄ alkyl);

OC(O)—(C₁-C₄ alkyl);

OC(O)NH—(C₁-C₄ alkyl);

OC(O)N(C₁-C₄ alkyl)₂;

OC(S)NH—(C₁-C₄ alkyl);

OC(S)N(C₁-C₄ alkyl)₂;

SH;

S—(C₁-C₄ alkyl);

S(O)—(C₁-C₄ alkyl);

S(O)₂—(C₁-C₄ alkyl);

SC(O)—(C₁-C₄ alkyl);

SC(O)O—(C₁-C₄ alkyl);

NH₂;

N(H)—(C₁-C₄ alkyl);

N(C₁-C₄ alkyl)₂;

N(H)C(O)—(C₁-C₄ alkyl);

N(CH₃)C(O)—(C₁-C₄ alkyl);

N(H)C(O)—CF₃;

N(CH₃)C(O)—CF₃;

N(H)C(S)—(C₁-C₄ alkyl);

N(CH₃)C(S)—(C₁-C₄ alkyl);

N(H)S(O)₂—(C₁-C₄ alkyl);

N(H)C(O)NH₂;

N(H)C(O)NH—(C₁-C₄ alkyl);

N(CH₃)C(O)NH—(C₁-C₄ alkyl);

N(H)C(O)N(C₁-C₄ alkyl)₂;

N(CH₃)C(O)N(C₁-C₄ alkyl)₂;

N(H)S(O)₂NH₂);

N(H)S(O)₂NH—(C₁-C₄ alkyl);

N(CH₃)S(O)₂NH—(C₁-C₄ alkyl);

N(H)S(O)₂N(C₁-C₄ alkyl)₂;

N(CH₃)S(O)₂N(C₁-C₄ alkyl)₂;

N(H)C(O)O—(C₁-C₄ alkyl);

N(CH₃)C(O)O—(C₁-C₄ alkyl);

N(H)S(O)₂O—(C₁-C₄ alkyl);

N(CH₃)S(O)₂O—(C₁-C₄ alkyl);

N(CH₃)C(S)NH—(C₁-C₄ alkyl);

N(CH₃)C(S)N(C₁-C₄ alkyl)₂;

N(CH₃)C(S)O—(C₁-C₄ alkyl);

N(H)C(S)NH₂;

NO₂;

CO₂H;

CO₂—(C₁-C₄ alkyl);

C(O)N(H)OH;

C(O)N(CH₃)OH:

C(O)N(CH₃)OH;

C(O)N(CH₃)O—(C₁-C₄ alkyl);

C(O)N(H)—(C₁-C₄ alkyl);

C(O)N(C₁-C₄ alkyl)₂;

C(S)N(H)—(C₁-C₄ alkyl);

C(S)N(C₁-C₄ alkyl)₂;

C(NH)N(H)—(C₁-C₄ alkyl);

C(NH)N(C₁-C₄ alkyl)₂;

C(NCH₃)N(H)—(C₁-C₄ alkyl);

C(NCH₃)N(C₁-C₄ alkyl)₂;

C(O)—(C₁-C₄ alkyl);

C(NH)—(C₁-C₄ alkyl);

C(NCH₃)—(C₁-C₄ alkyl);

C(NOH)—(C₁-C₄ alkyl);

C(NOCH₃)—(C₁-C₄ alkyl);

CN;

CHO;

CH₂OH;

CH₂O—(C₁-C₄ alkyl);

CH₂NH₂;

CH₂N(H)—(C₁-C₄ alkyl);

CH₂N(C₁-C₄ alkyl)₂;

aryl;

heteroaryl;

cycloalkyl; and

heterocyclyl.

In some cases, a ring substituent may be shown as being connected to the ring by a bond extending from the center of the ring. The number of such substituents present on a ring is indicated in subscript by a number. Moreover, the substituent may be present on any available ring atom, the available ring atom being any ring atom which bears a hydrogen which the ring substituent may replace. For illustrative purposes, if variable Rx were defined as being:

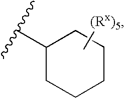

this would indicate a cyclohexyl ring bearing five Rx substituents. The Rx substituents may be bonded to any available ring atom. For example, among the configurations encompassed by this are configurations such as:

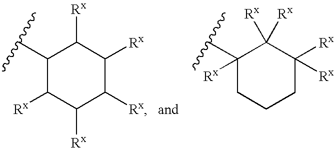

These configurations are illustrative and are not meant to limit the scope of the invention in any way.

### Biological Activity

The determination of inhibition towards different metalloproteases of the heterobicyclic metalloprotease inhibiting compounds of the present invention may be measured using any suitable assay known in the art. A standard in vitro assay for measuring the metalloprotease inhibiting activity is described in Examples 1700 to 1706. The heterobicyclic metalloprotease inhibiting compounds show activity towards MMP-3, MMP-8, MMP-12, MMP-13, ADAMTS-4 and/or ADAMTS-5.

The heterobicyclic metalloprotease inhibiting compounds of the invention have an MMP-3 and/or MMP-13 inhibition activity (IC₅₀ MMP-3 and/or IC₅₀ MMP-13) ranging from below 3 nM to about 20 μM, and typically, from about 3 nM to about 2 μM. Heterobicyclic metalloprotease inhibiting compounds of the invention desirably have an MMP inhibition activity ranging from about 3 nM to about 100 nM. Table 1 lists typical examples of heterobicyclic metalloprotease inhibiting compounds of the invention that have an MMP-3 and/or MMP-13 activity from 3 nM to 100 nM (Group A) and from 101 nM to 20 μM (Group B).

TABLE 1

| Group | Examples |
|---|---|
| | Summary of MMP-3 Activity for Compounds |
| A | 8, 21, 25 |
| B | 1, 4, 5, 6, 7, 9, 13, 15, 19, 20, 22, 23, 26, 27, 28, 29 |
| | Summary of MMP-13 Activity for Compounds |
| A | 23 |
| B | 4, 5, 6, 7, 8, 9, 13, 15, 19, 20, 21, 22, 25, 26, 27, 28, 29 |

The synthesis of metalloprotease inhibiting compounds of the invention and their biological activity assay are described in the following examples which are not intended to be limiting in any way.

Schemes

In some embodiments the compounds of Formula (I) are synthesized by the general methods shown in Scheme 1 to Scheme 10.

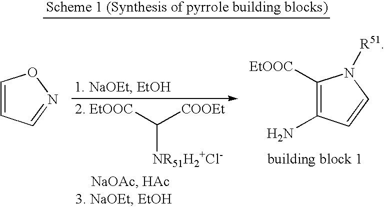

Commercially available isoxazole is treated at 0° C. with sodium ethoxide in ethanol, followed by the addition of a suitable amino malonate derivative (e.g. 2-amino-malonic acid diethyl ester). The intermediates are then treated with sodium ethoxide in ethanol at room temperature and the desired building blocks 1 (e.g. 3-amino-1H-pyrrole-2-carboxylic acid ethyl ester) are obtained after purification.

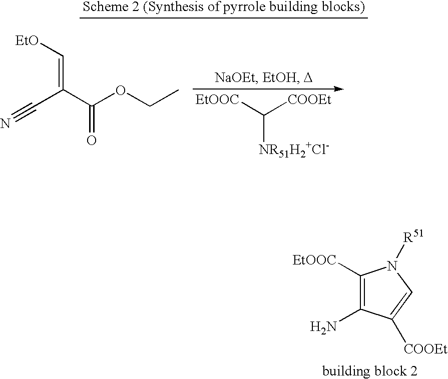

Commercially available 2-cyano-3-ethoxy-acrylic acid ethyl ester is heated at reflux with sodium ethoxide and a suitable amino malonate derivative (e.g. 2-amino-malonic acid diethyl ester) to afford the desired building blocks 2 (e.g. 3-amino-1H-pyrrole-2,4-dicarboxylic acid diethyl ester) after purification

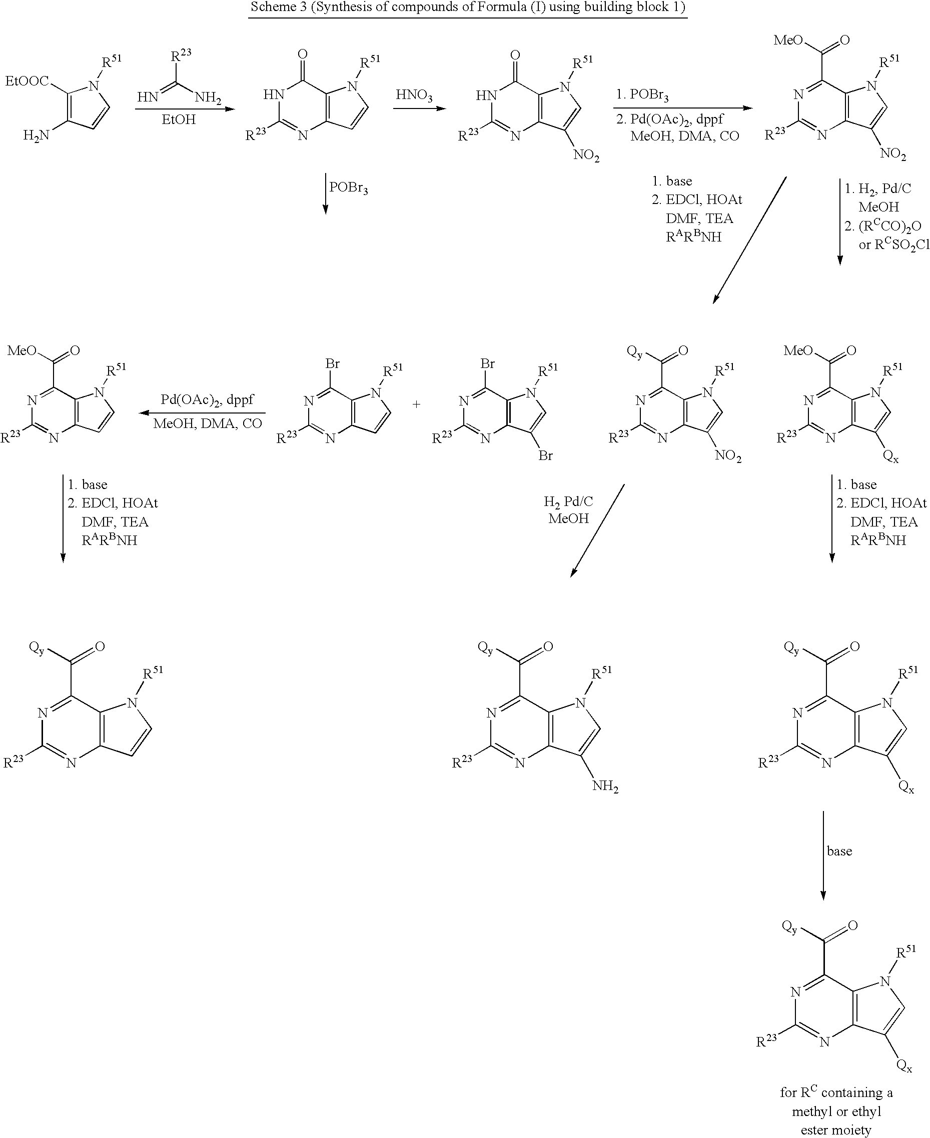

Building blocks 1 (e.g. 3-amino-1H-pyrrole-2-carboxylic acid ethyl ester) are condensed (e.g. EtOH/reflux) with a suitable amidine derivative (e.g. formamidine) to give the corresponding 9-deazahypoxanthine derivatives (Scheme 3).

These intermediates are then converted into the corresponding mono- and dibromo derivatives using a suitable reagent (e.g. POBr₃/80° C.). The resulting mixtures of bromides are heated to (e.g. 80° C.) with a suitable catalyst (e.g. Pd(OAc)₂, dppf) and base (e.g. Et₃N) under a carbon monoxide atmosphere in a suitable solvent (e.g. MeOH) to give the corresponding bicyclic methylesters after purification. Saponification of the ester with base (e.g. aqueous KOH) and coupling of the resulting acid derivatives using an activated acid method (e.g. EDCI, HOAt, DMF, base) with R^{A}R^{B}NH (e.g. 6-aminomethyl-4H-benzo[1,4]oxazin-3-one) affords the desired final products with Qₓ=H (Scheme 3).

The 9-deazahypoxanthine derivatives are converted to their 7-nitro derivatives by nitration (e.g. concentrated HNO₃/0° C. to room temperature). Heating of these compounds in neat POBr₃ affords the corresponding 4-bromo,7-nitro derivatives after aqueous workup. The 4-bromo,7-nitro derivatives are heated (e.g. 80° C.) with a suitable catalyst (e.g. Pd(OAc)₂, dppf) and base (e.g. Et₃N) under a carbon monoxide atmosphere in a suitable solvent (e.g. MeOH) to give the corresponding 7-nitro bicyclic methylesters after purification. Saponification of the ester moiety with base (e.g. aqueous KOH) and coupling of the resulting acid derivatives using an activated acid method (e.g. EDCI, HOAt, DMF, base) with R^{A}R^{B}NH (e.g. 6-aminomethyl-4H-benzo[1,4]oxazin-3-one) affords the desired final products with Qₓ=NO₂. Catalytic hydrogenation of these compounds affords the desired final products with Qₓ═NH₂ (Scheme 3).

Catalytic hydrogenation of the nitro group of the 7-nitro bicyclic methylester derivatives, yields the corresponding amine intermediates, which are reacted with suitable, sulfonyl chlorides R^{C}SO₂Cl or anhydrides (R^{C}CO)₂O to afford the desired compounds after purification. Saponification of the ester moiety with base (e.g. aqueous KOH) and coupling of the resulting acid derivatives using an activated acid method (e.g. EDCI, HOAt, DMF, base) with R^{A}R^{B}NH (e.g. 6-aminomethyl-4H-benzo[1,4]oxazin-3-one) affords the desired final products (Scheme 3). In case R^{c} contains methyl or ethyl ester moieties, the desired final compounds having free acid residues are obtained after saponification of the esters with base (e.g. aqueous KOH) and subsequent purification.

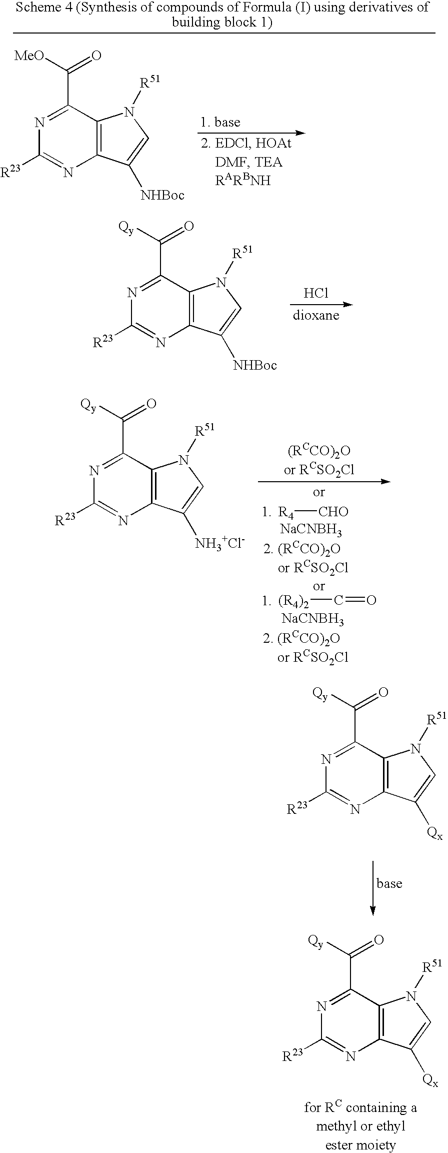

The ester moiety of the compounds from Scheme 3 with Qₓ=NHBoc is removed with base (e.g. aqueous KOH) and the resulting acids are coupled with R^{A}R^{B}NH (e.g. 6-aminomethyl-4H-benzo[1,4]oxazin-3-one) using an activated acid method (e.g. EDCI, HOAt, DMF, base) to afford the desired compounds (Scheme 4). Cleavage of the Boc protecting group with acid (e.g. HCl, dioxane) affords the desired hydrochloride salts. Treatment of these salts in a suitable solvent (e.g. pyridine) with suitable sulfonyl chlorides R^{C}SO₂Cl or anhydrides (R^{C}CO)₂O affords the desired final compounds after purification. In case R^{c} contains methyl or ethyl ester moieties, the desired final compounds having free acid residues are obtained after saponification of the esters with base (e.g. aqueous KOH) and subsequent purification.

Treating the hydrochloride salts with a suitable aldehyde or ketone using reductive amination (e.g. NaCNBH₃ or NaBH(OAc)₃), followed by the treatment of the secondary amine with suitable sulfonyl chlorides R^{C}SO₂Cl or anhydrides (R^{C}CO)₂O affords the desired final compounds after purification. In case R^{c} contains methyl or ethyl ester moieties, the desired final compounds having free acid residues are obtained after saponification of the esters with base (e.g. aqueous KOH) and subsequent purification.

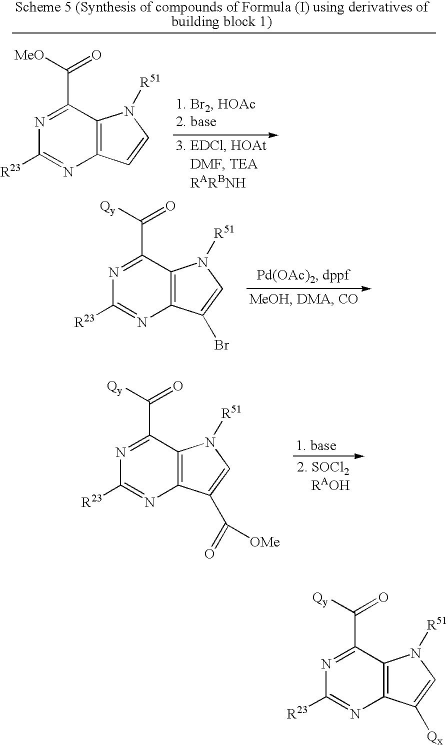

Bromination of 4-methyl ester derivatives with bromine (e.g. Br₂, HOAc), followed by saponification of the ester moiety with base (e.g aqueous KOH) and coupling of the free acids with R^{A}R^{B}NH (e.g. 6-aminomethyl-4H-benzo[1,4]oxazin-3-one) using an activated acid method (e.g. EDCI, HOAt, DMF, base) affords the desired compounds after purification (Scheme 5). The bromides are heated (e.g. 80° C.) with a suitable catalyst (e.g. Pd(OAc)₂, dppf) and base (e.g. Et₃N) under a carbon monoxide atmosphere in a suitable solvent (e.g. MeOH) to give the corresponding 7-methyl esters after purification. Saponification of the 7-methyl ester moiety with base at elevated temperatures (e.g. LiOH, 70-100° C.) yields the free acids. Treatment of the free acids with thionylchloride and a suitable alcohol yields the corresponding compounds with Qₓ=C(O)OR^{A} after purification.

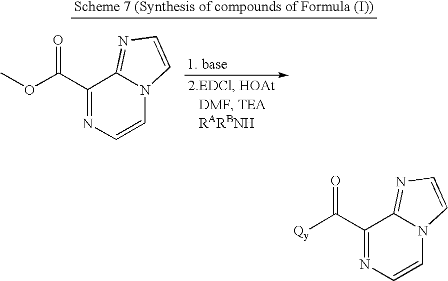

Commercially available imidazo[1,2-a]pyrazine-8-carboxylic acid methyl ester is treated with base (e.g. aqueous KOH) and the free acid is coupled with R^{A}R^{B}NH (e.g. 6-aminomethyl-4H-benzo[1,4]oxazin-3-one)) using an activated acid method (e.g. EDCI, HOAt, DMF, base) to afford the desired final products after purification with Qₓ=H (Scheme 7).

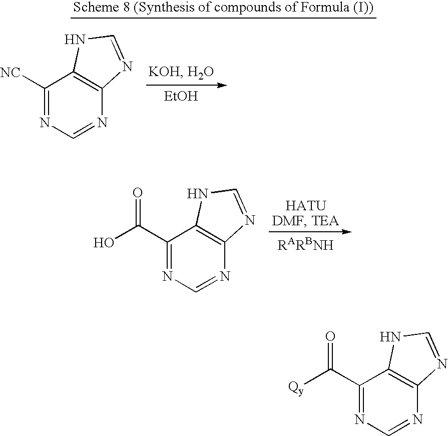

Commercially available 7H-purine-6-carbonitrile is heated (e.g. 100° C.) with base (e.g. KOH) in a suitable solvent (e.g. EtOH) to give the corresponding acid derivative after purification. Coupling of the free acid with R^{A}R^{B}NH (e.g. 6-aminomethyl-4H-benzo[1,4]oxazin-3-one)) using an activated acid method (e.g. HATU, DMF, base) affords the desired final products after purification (Scheme 8).

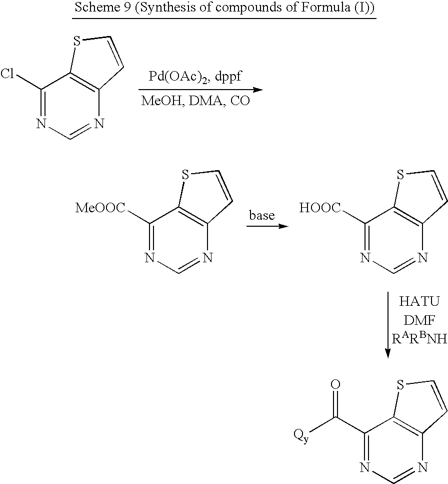

Commercially available 4-chloro-thieno[3,2-d]pyrimidine is heated (e.g. 80° C.) with a suitable catalyst (e.g. Pd(OAc)₂, dppf) and base (e.g. TEA) under a carbon monoxide atmosphere in a suitable solvent (e.g. MeOH) to give the corresponding ester derivatives after purification.

Saponification of the ester with base (e.g. aqueous KOH) and coupling of the resulting acid derivative with R^{A}R^{B}NH (e.g. 6-aminomethyl-4H-benzo[1,4]oxazin-3-one)) using an activated acid method (e.g. EDCI, HOAt, DMF, base) affords the desired final products after purification (Scheme 9) with Qₓ=H.

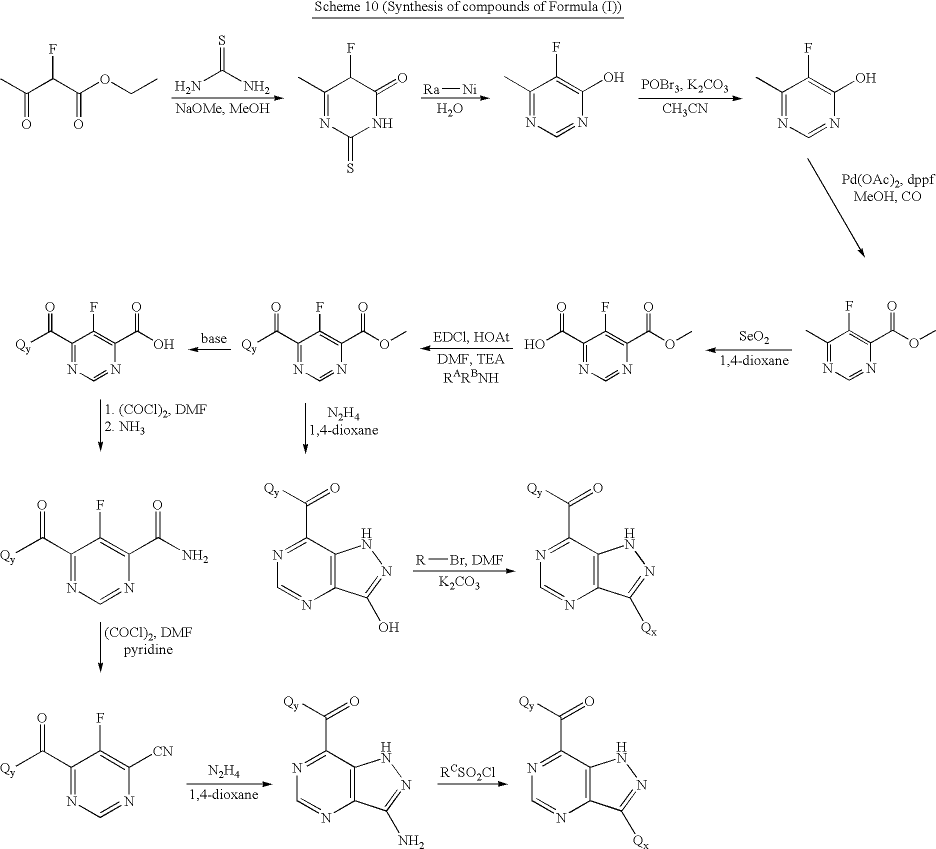

Commercially available 2-fluoro-3-oxo-butyric acid ethyl ester and thiourea are treated at elevated temperature (e.g. 100° C.) with base (e.g. NaOMe) in a suitable solvent (e.g. MeOH) to afford the corresponding fluoro pyrimidinone derivative. Removal of the sulphur with a catalyst (e.g. Raney-nickel) at elevated temperature in a suitable solvent (e.g. H₂O; 100° C.) afforded the desired fluoro pyrimidine derivative. The corresponding bromo derivative is obtained by heating the precursor in a suitable solvent (e.g. CH₃CN) with base (e.g. K₂CO₃) and POBr₃. The bromo derivative is then heated (e.g. 80° C.) with a suitable catalyst (e.g. Pd(OAc)₂, dppf) and base (e.g. Et₃N) under a carbon monoxide atmosphere in a suitable solvent (e.g. MeOH) to give the corresponding fluoro-pyrimidine-carboxylic acid methyl ester after purification. Oxidation of the methyl group with a suitable reagent (e.g. seleldioxide) in a suitable solvent (e.g. dioxane) at elevated temperature (e.g. 120° C.) in a sealed vessel affords the desired fluoro-pyrimidine monoacid/monoester.

Coupling of the acid derivative using an activated acid method (e.g. EDCI, HOAt, DMF, base) with R^{A}R^{B}NH (e.g. 3-chloro-4-fluoro benzylamine) affords the desired products after purification (Scheme 10). Treatment of these derivatives with hydrazine in a suitable solvent (e.g. 1,4-dioxane) affords the desired 3-hydroxy-1H-pyrazolo[4,3-d]pyrimidin derivatives. Coupling of these derivatives with bromides (e.g. 4-fluorobenzylbromide) in the presence of base (e.g. K₂CO₃) in a suitable solvent (DMF) affords the desired final compounds after purification (Scheme 10).

Saponification of the remaining ester moiety with base (e.g. aqueous KOH) affords the corresponding free acid derivatives. The free acid derivatives are converted to the corresponding amides via the formation of their acid chlorides using suitable conditions (e.g. oxalyl chloride, DMF, 0° C.), followed by treatment with anhydrous ammonia (0.5 M in 1,4-dioxane) and subsequent purification. Dehydratization to the corresponding nitrile under suitable conditions (e.g. oxalyl chloride, DMF, pyridine, 0° C.) affords the desired derivatives after workup. Treatment of these derivatives with hydrazine in a suitable solvent (e.g. 1,4-dioxane) affords the desired 3-amino-1H-pyrazolo[4,3-d]pyrimidin derivatives. Reaction of the free amine with suitable sulfonyl chlorides R^{C}SO₂Cl affords the desired final compounds after purification (Scheme 10).

### PREPARATIVE EXAMPLE 1

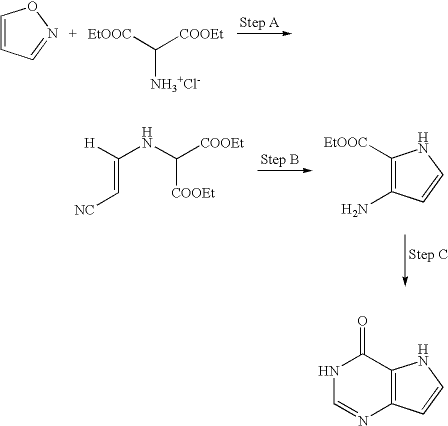

### Step A

Commercially available isoxazole (25 g) was dissolved in EtOH (100 ml) and the mixture cooled to 0° C. At 0° C. a solution of 21% NaOEt in EtOH (124 ml) was slowly added to keep the temperature<8° C. After the complete addition, the mixture was stirred in the ice bath for another 30 min (precipitate formed). Then acetic acid (6.9 ml), sodium acetate (20.5 g) and the HCl salt of diethyl malonate (48 g) were added. The mixture was stirred for 48 h and allowed to reach room temperature. The solvent was removed and the residue portioned between CH₂Cl₂ and H₂O. The organic phase was separated, dried over MgSO₄ and filtered through a plug of silica. The plug was washed with CH₂Cl₂ until all product eluted. The filtrate was evaporated to afford the title compound as orange oil (MH⁺=227).

### Step B

The crude title compound from Step A above was dissolved in EtOH (420 ml). The mixture was treated with a solution of 21% NaOEt in EtOH (81 ml) and stirred at room temperature for 3 days. After the addition of acetic acid (15 ml), the solvent was removed. The residue was dissolved in CH₂Cl₂ and washed with NaHCO₃ (pH˜7). The organic phase was dried over MgSO₄ and filtered through a plug of silica. The plug was washed with CH₂Cl₂ until all product eluted. The filtrate was concentrated and the residue dried in HV to afford the title compound derivative as an orange syrup (23 g; 65%; MH⁺=155).

### Step C

The title compound from Step B above (23 g) was dissolved in EtOH (210 ml) and formamidine acetate (23.3 g) added. The mixture was heated at 100-105° C. oil-bath temperature for 16 h. The mixture was cooled to room temperature and the precipitate collected by filtration. The precipitate was then washed with EtOH until the washing solution was colorless. The precipitate was then dried in HV to afford the product as a grey solid (15.3 g; 75%; MH⁺=136).

### PREPARATIVE EXAMPLE 2

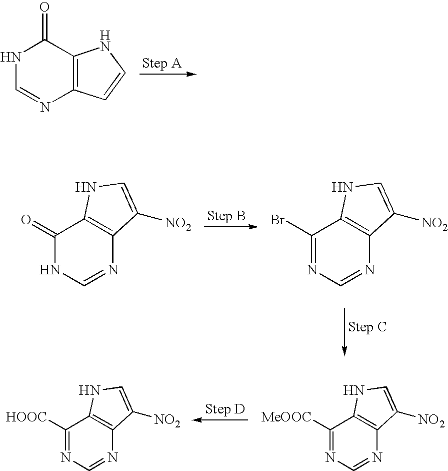

### Step A

A solution of HNO₃ was prepared by mixing 90% HNO₃ (8 ml) and 65% HNO₃ (4 ml). The solution was cooled to 0° C. and the title compound from Preparative Example 1 (4 g) added in portions. After the complete addition, conc. H₂SO₄ (13.6 ml) was slowly added as to keep the internal temperature below 12° C. After the complete addition, the mixture was stirred in the ice bath for 2 h to become a clear, yellow solution. This solution was then poured onto a mixture of 30 g ice and 60 ml H₂O. A precipitate was formed and allowed to stand for 30 min. The precipitate was collected by filtration, washed with H₂O (160 ml) and dried in HV to afford the title compound as a yellow solid (4.78 g, 89%). ¹H-NMR (DMSO-d₆) δ 8.10 (s, 1H), 8.52 (d, 1H), 12.58 (s, 1H), 13.50 (s, 1H)

### Step B

The title compound from Step A above (4.78 g) was grinded in a mortar and added at ˜110-115° C. in portions to a solution of neat POBr₃ (40 g). The mixture was then stirred at this temperature overnight. The mixture was cooled to room temperature to become a solid. The solid was carefully quenched in an ice bath with ice water (450 ml). Then solid NaHCO₃ was carefully added until pH˜8. The mixture was extracted with EtOAc (6×400 ml). The organic phase was dried over MgSO₄, filtered and the solvents removed to afford a portion of the title compound (1.3 g, 20%, MH⁺=243/245). Another portion was obtained by adding conc. HCl to the aqueous solution until pH˜1-2. The precipitate was collected by filtration, washed with H₂O and dried in HV to afford a mixture (2.7 g) of the title compound (70%) and unreacted starting material (30%).

### Step C

A slurry of the mixture of the title compound and unreacted starting material from Step B above (2.7 g) in DMA (50 ml) and MeOH (75 ml) was treated with triethylamine (3.5 ml) and sonicated for 25 min while a stream of N₂ was passed through the mixture. After the sonication was completed, the Pd(OAc)₂ (130 mg) and 1,1′-Bis(diphenylphosphino)ferrocene (252 mg) were added. The mixture was then carbonylated using an autoclave until all the bromo starting material was consumed (CO-pressure: 6.5 bar; temperature: 80° C.). The reaction mixture was filtered and the solid material washed with MeOH. The combined filtrate was evaporated in vacuo and in HV to remove traces of DMA. The black residue was dissolved in MeOH and treated with silica. This slurry was evaporated and the coated silica put onto a column equilibrated with CH₂Cl₂. The column was developed with CH₂Cl₂ (800 ml) and then with CH₂Cl₂/MeOH (800 ml) to elute the reddish unpolar impurities. Then the column was developed with CH₂Cl₂/MeOH (95:5) to afford the title compound as orange solid (1000 mg, 41%, MH⁺=223).

### Step D

The title compound from Step C above (517 mg) was dissolved in THF (40 ml) and H₂O (20 ml) and treated with LiOH×H₂O (391 mg). The mixture was then stirred at room temperature for 90 Min. The solvents were evaporated and the residue treated with 1 M HCl (15 ml). The precipitate was collected by filtration, washed with 1 M HCl and H₂O and then dried in HV to afford the title compound as beige solid (429 mg; 89%; MH⁺=209).

### PREPARATIVE EXAMPLE 3

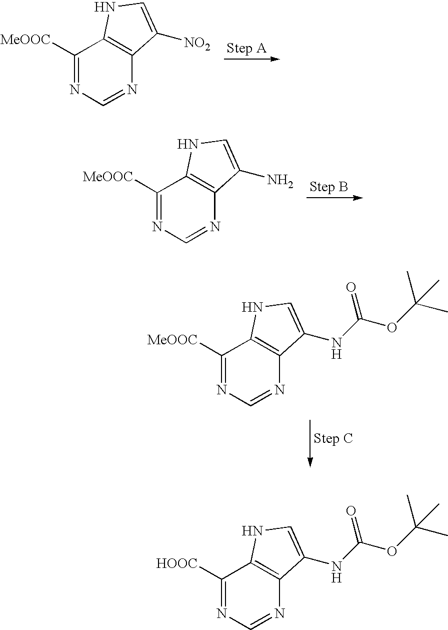

### Step A

The title compound from Preparative Example 2 Step C (832 mg) was dissolved in MeOH (80 ml) and treated with 10% Pd/C (300 mg). The mixture was hydrogenated for 30 min and then filtered. The catalyst was washed with MeOH and the combined filtrate evaporated to afford the desired compound as a red glass (719 mg, quant.; MH⁺=193).

### Step B

The crude title compound from Step A above (540 mg) was dissolved in THF (12 ml) and CH₃CN (12 ml) and triethylamine (0.4 ml) added. After the addition of Boc₂O (590 mg), the mixture was stirred at room temperature overnight. The mixture was evaporated and the residue suspended in CH₂Cl₂/MeOH (98:2). This slurry was put onto a silica column and the column was developed with CH₂Cl₂/MeOH (98:2) to afford the title compound as yellow solid (300 mg, 32%; MH⁺=293).

### Step C

The title compound from Step B above (150 mg) was dissolved in THF (4.3 ml), CH₃CN (4.3 ml) and H₂O (4.3 ml). The clear solution was treated with 1 M KOH (0.77 ml,) and stirred at room temperature for 1 h. The mixture was evaporated and dried in HV to remove traces of H₂O to afford the title compound as its potassium-salt (162 mg; quant.; MH⁺=279)

### PREPARATIVE EXAMPLE 4

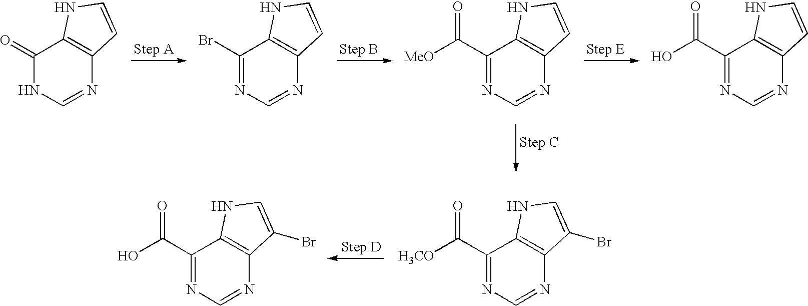

### Step A

The title compound from Preparative Example 1 (1.96 g) was added at 70-80° C. to a solution of POBr₃ (16 g). The mixture was stirred at this temperature for 2 h 15 Min and then cooled to room temperature. To the solid material was carefully added a mixture of sat NaHCO₃ and ice until the pH of the aqueous phase was pH˜8. The aqueous phase was then extracted with CHCl₃/MeOH (9:1; 2×300 ml), with EtOAc/MeOH (9:1; 2×300 ml) and EtOAc/THF (9:1; 2×300 ml). Each of the extracts was washed with brine, dried over MgSO₄ filtered and the solvents removed to afford the title compound as yellow solid (1.37 g; 48%; MH⁺=197/199).

### Step B

The title compound from Step A above (1.37 g) was dissolved in DMA (30 ml) and MeOH (45 ml) and TEA (2 ml) added. The mixture was then sonicated for 15 Min while a stream of argon was bubbled through the solution. Then 1,1′-Bis-(diphenylphosphino)-ferrocen (95 mg) and Pd(OAc)₂ (48 mg) were added and the mixture carbonylated (7 bar CO) in a pressure reactor at 80° C. for 2 d. The reaction mixture was then filtered and the filter washed with MeOH. The combined filtrate was evaporated, the residue dissolved/suspended in MeOH and silica added. The MeOH was evaporated and the coated silica loaded onto a silica column equilibrated with CH₂Cl₂. The column was then developed using a gradient (CH₂Cl₂->CH₂Cl₂/MeOH (95:5). Fractions containing the product were collected and the solvents evaporated to afford the title compound as a reddish solid (1.19 g; 97%; MH⁺=178).

### Step C

The title compound from Step B above (616 mg) was dissolved in acetic acid (96 ml). Then bromine (192 μl) was slowly added at room temperature with stirring. After 1 h at room temperature another batch of bromine (30 μl) was added and stirring at room temperature was continued for 30 Min. Then the acetic acid was evaporated and the residue dried in HV to afford the title compound as an orange solid (MH⁺=255/257).

### Step D

The crude title compound from Step C above was suspended in THF (70 ml) and H₂O (30 ml). After the addition of LiOH×H₂O (245 mg), the mixture was stirred at room temperature for 1 h. Another batch of LiOH×H₂O (60 mg) was added and stirring was continued for 45 Min. Then 1 M HCl (9 ml) was added and the solvents evaporated. The residue was suspended in THF (2×20 ml) and each time the solvents evaporated. The residue was then dried in HV to afford the title compound as off white solid (M⁺=241/243).

### PREPARATIVE EXAMPLE 5

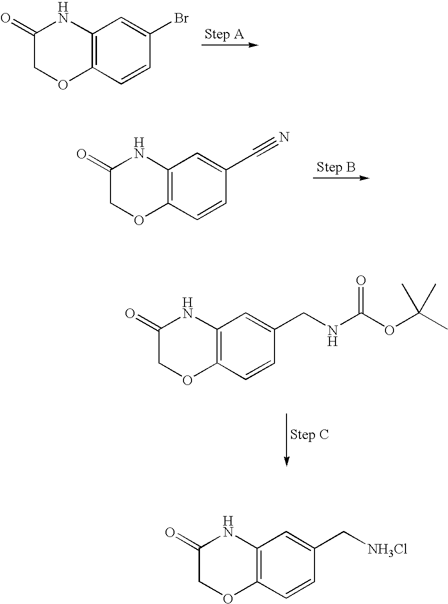

### Step A

A degassed suspension of commercially available 6-Bromo-4H-benzo[1,4]oxazin-3-one (8.39 g), Zn(CN)₂ (3.46 g) and Pd(PPh₃)₄ (2.13 g) in DMF (70 mL) was stirred in a oil bath (80° C.) overnight. The mixture was cooled to room temperature and then poured into water (500 mL). The precipitate was collected by suction, air dried, washed with pentane, dissolved in CH₂Cl₂/MeOH (1:1), filtered through an silica pad and concentrated to yield a yellow solid (5.68 g, 89%; MH⁺=175).

### Step B

To an ice cooled solution of the title compound from Step A above (5.6 g), di-tert-butyl dicarbonate (14.06 g) and NiCl₂.6H₂O (1.53 g) in MeOH, NaBH₄ (8.51 g) was added in portions. The mixture was vigorously stirred for 1 h at 0° C. and 1 h at room temperature. After the addition of diethylenetriamine (3.5 mL) the mixture was concentrated, diluted with EtOAc, washed subsequently with 1N HCl, saturated aqueous NaHCO₃ and saturated aqueous NaCl, dried (MgSO₄), concentrated to afford the title compound as an off white solid (7.91 g, 88%; M+Na⁺=397).

### Step C

The title compound from Step B above (7.91 g) was dissolved in a 4M solution of HCl in 1,4-dioxane (120 mL), stirred for 14 h, concentrated, suspended in Et₂O, filtered and dried to afford the title compound as an off-white solid (5.81 g, 96%; M-NH₃Cl⁺=162).

### PREPARATIVE EXAMPLE 6

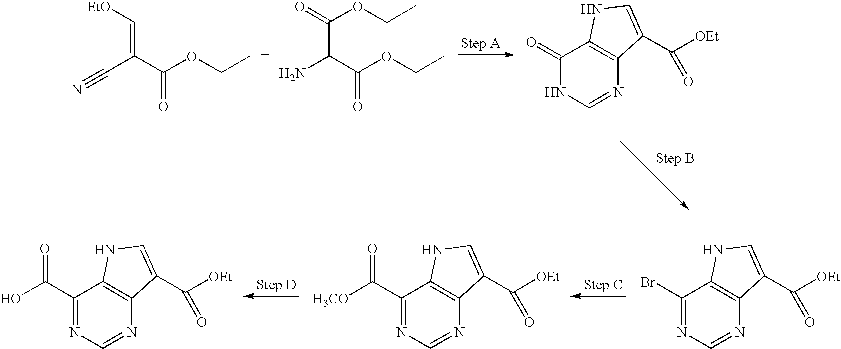

### Step A

To a solution of commercial available ethyl 2-cyano-3-ethoxyacrylate (8.46 g) in abs. ethanol (35 ml) was added commercial available diethyl amino malonate hydrochloride (10.58 g). The resulting mixture was stirred at room temperature for 10 min. Then a solution of sodium ethanolate in ethanol (40.53 ml, 2.7 M) was added. The mixture was heated to reflux for 16 h. After cooling to room temperature formamidine acetate (10.51 g) was added. To the vigorously stirred mixture acetic acid (3.46 ml) was added and the mixture was heated to reflux for 68 h. The mixture was cooled to room temperature and filtered. The resulting solid was suspended in ethanol (300 ml). After filtration the obtained solid was dried to afford the crude title compound as grey solid, which was used without further purification. (8.6 g: 83%; MH⁺=208).

### Step B

To a heated solution of POBr₃ (100 g) the title compound from Step A above (14.5 g), was added. The suspension was heated to 90° C. for 1 h. After cooled to room temperature, the resulting residue was added in small portions to an ice cooled saturated aqueous solution of NaHCO₃ (3.5 l). After stirring for 30 min. the suspension was filtered. The resulting solid was washed with water and dried to afford the title compound as a off-white solid (15.2 g; 80%; MH⁺=270/272).

### Step C

The title compound from Step B above (5 g), Pd(OAc)₂ (126 mg), 1,1′-Bis(diphenyl-phosphino)ferrocene (416 mg) and NEt₃ (5.2 ml) were dissolved in dry DMA MeOH (7:3, 100 ml) and stirred at 80° C. under a carbon monoxide atmosphere at 7 bar overnight. The mixture was concentrated, absorbed on silica and purification by chromatography (silica, CH₂Cl₂/MeOH) afforded the title compound as off-white solid (3.4 g,; 72%; MH⁺=250).

### Step D

To a solution of the title compound from Step C above (85 mg) in THF (60 ml) was added aqueous LiOH (875 mg in 30 ml). The resulting mixture was stirred at room temperature for 1 h, adjusted to pH 2 and filtrated. The resulting solid was washed with water to give a colourless solid, which was used without further purification (2.25 g; 96%; MH⁺=236).

### PREPARATIVE EXAMPLE 7

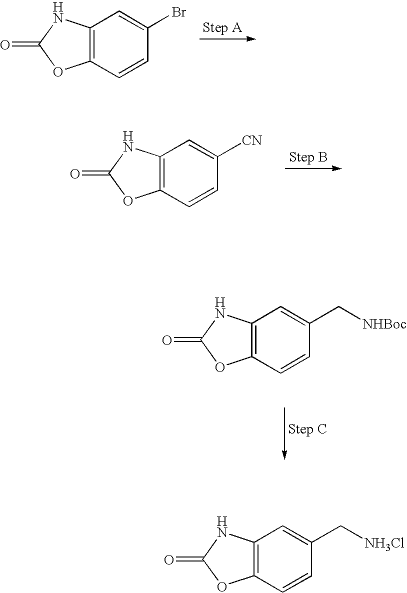

### Step A

Commercially available 5-Bromo-3H-benzooxazol-2-one (1 g) was dissolved in DMF (15 ml) and Zn(CN)₂ (1.09 g) added. The mixture was sonicated for 5 Min while a stream of nitrogen was bubbled through the solution. After the addition of Pd[P(Ph)₃]₄ (0.54 g), the mixture was heated at 100° C. oil bath temperature for 18 h. The solvents were evaporated and the residue purified by chromatography on silica using EtOAc/cyclohexane (20:80->50:50) to afford the title compound as white solid (674 mg; 91%; MH⁺=161).

### Step B

The title compound from Step A above (300 mg) was dissolved in MeOH (40 ml) and NiCl₂×6H₂O (44.4 mg) and Boc₂O (816 mg) added. The mixture was cooled to 0° C. and NaBH₄ (495 mg) was added in portions. After the addition was completed, the mixture was stirred overnight and allowed to reach room temperature. The solvents were evaporated and the residue dissolved in EtOAc. The organic phase was washed with sat. NaHCO₃, dried over MgSO₄, filtered and the solvents evaporated. The residue was purified by chromatography on silica using EtOAc/cyclohexane (20:80) to afford the title compound as a white foam (428 mg; 87%; MH⁺=265).

### Step C

The title compound from Step B above (428 mg) was dissolved in 4 M HCl in dioxane (8 ml) and the mixture stirred at room temperature for 2 h. The solvents were removed and the residue dried in HV to afford the title compound as orange solid (347 mg; quant.; MH⁺=165).

### PREPARATIVE EXAMPLE 8

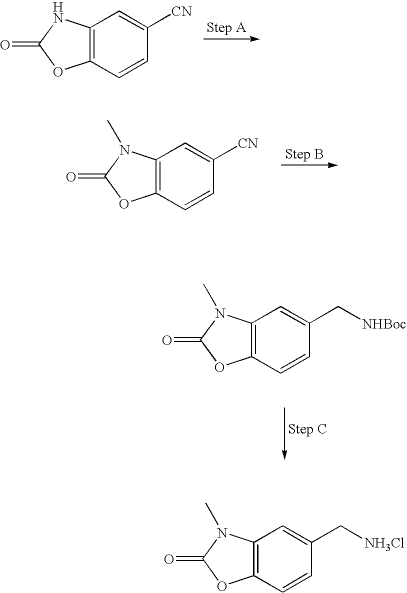

### Step A

The title compound from Preparative Example 7 Step A (374 mg) was dissolved in DMF (30 ml) and NaH (112 mg) added. The mixture was stirred at room temperature for 2 h, CH₃I (358 μl) added and stirring at room temperature was continued overnight. The solvents were evaporated and the residue dissolved in EtOAc. The organic phase was washed with H2O, dried over MgSO₄, filtered and the solvents evaporated to afford the title compound as pale yellow solid (398 mg; 99%; MH⁺=175).

### Step B

The title compound from Step A above (398 mg) was treated with NiCl₂×6H₂O (52 mg) and NaBH₄ (582 mg) in the presence of Boc₂O (960 mg) as described in Preparative Example 7 Step B to afford the title compound (546 mg; 89%; MH⁺=279).

### Step C

The title compound from Step B above (546 mg) was treated with 4 M HCl/dioxane (10 ml) as described in Preparative Example 7 Step C to afford the title compound as yellow solid (420 mg; quant.; MH⁺=179).

### PREPARATIVE EXAMPLE 9

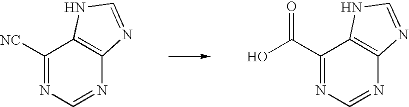

Commercially available 7H-purine-6-carbonitrile (50 mg) was mixed with KOH (49 mg) and treated with EtOH (4 ml) and H2O (1 ml). The mixture was heated at 100° C. oil-bath temperature overnight. After the addition of another batch of KOH (92 mg), the mixture was again heated at 100° C. overnight. The mixture was cooled and 1 M HCl (2 ml) added. The precipitate was then collected by filtration to afford the title compound (91 mg; quant.; MH⁺=165).

### PREPARATIVE EXAMPLE 10

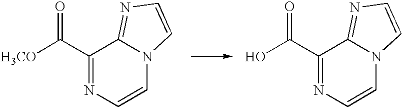

Commercially available Imidazo[1,2-a]pyrazine-8-carboxylic acid methyl ester (337 mg) was dissolved in 1,4-dioxane (25 ml) and H₂O (58 ml) and treated with 1 M NaOH (2.4 ml). The mixture was stirred at room temperature for 45 Min and the solvents evaporated to afford the crude title compound (MH⁺=164).

### PREPARATIVE EXAMPLE 11

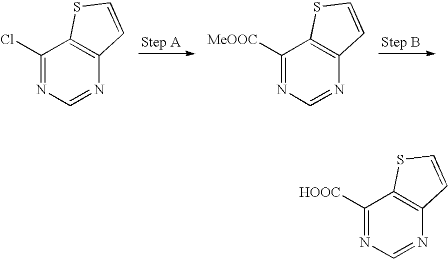

### Step A

Commercially available 7-chloro-benzo[b]thiophene (400 mg), Pd(OAc)₂ (16 mg), dppf (51 mg) and NEt₃ (641 μl) were dissolved in dry DMA/MeOH (3:2, 50 ml) and stirred at 80° C. under a carbon monoxide atmosphere at 7 bar over the weekend. The mixture was concentrated, absorbed on silica and purification by chromatography (silica, CH₂Cl₂/MeOH) afforded the title compound as colourless solid (180 mg; 40%; MH⁺=195).

### Step B

To a solution of the title compound from Step A above (85 mg) in THF (4 ml) was added aqueous LiOH (52 mg in 1 ml H₂O). The resulting mixture was stirred at room temperature for 3 h, adjusted to pH 2 and filtrated. The resulting solid was washed with water to give an off-white solid (80 mg; 99%; MH⁺=181).

### PREPARATIVE EXAMPLE 12

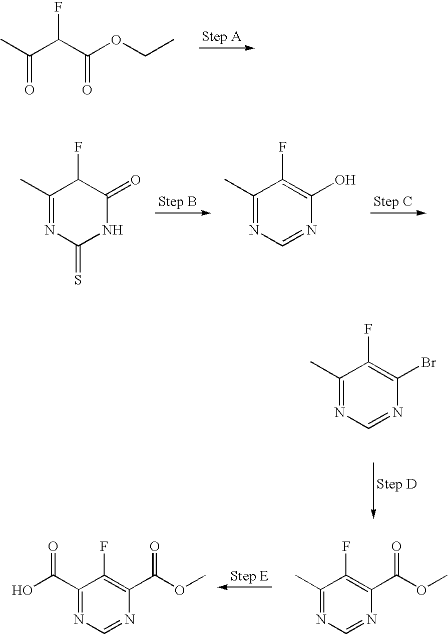

### Step A

A mixture of NaOMe (5.40 g), thiourea (5.35 g) and commercially available 2-fluoro-3-oxo-butyric acid ethyl ester (6.27 ml) in anhydrous MeOH (50 ml) was stirred at 100° C. (temperature of the oil bath) for 5½ h and then allowed to cool to room temperature. The obtained beige suspension was concentrated and diluted with H₂O (50 ml). To the resulting aqueous solution was added concentrated HCl (9 ml). The formed precipitate was collected by filtration and washed with H₂O (100 ml) to afford the title compound as a pale beige solid (5.6 g, 70%; MH⁺ 161).

### Step B

A suspension of the title compound from Step A above (5.6 g) and Raney®-nickel (50% slurry in H₂O, 8 ml) in H₂O (84 ml) was heated to reflux for 16 h. The mixture was allowed to cool to room temperature and then filtered. The filter cake was washed successively with MeOH and EtOAc and the combined filtrates were concentrated. The obtained viscous oily residue was diluted with EtOAc and concentrated to afford the title compound as a reddish solid (3.6 g, 80%; MH⁺=129).

### Step C

A mixture of the title compound from Step B above (3.6 g), K₂CO₃ (11.6 g) and POBr₃ (24.0 g) in anhydrous CH₃CN (200 ml) was heated to reflux for 19 h, cooled to room temperature and concentrated. A mixture of ice (180 g) and H₂O (30 ml) was added and the mixture was stirred for 30 min. The aqueous mixture was extracted with CHCl₃ (2×150 ml) and EtOAc (2×150 ml) and the combined organic extracts were washed with saturated aqueous NaCl, dried (MgSO₄), filtered and concentrated to afford the title compound as a yellow liquid (3.15 g, 58%; MH⁺=191/193).

### Step D

Under a carbon monoxide atmosphere (7 bar) a mixture of the title compound from Step C above (2.91 g), Pd(OAc)₂ (142 mg), 1,1′-bis-(diphenylphosphino)ferrocene (284 mg) and Et₃N (4.2 ml) in anhydrous DMA/MeOH (1:1, 150 ml) was heated at 80° C. for 17 h. The mixture was cooled to room temperature, concentrated, absorbed on silica (500 mg) and purified by chromatography (silica, cyclohexane/EtOAc) to afford the title compound as a beige solid (1.53 g, 59%; MH⁺=171).

### Step E

The title compound from Step D above (473 mg) was dissolved in 1,4-dioxane (17.4 ml) and selendioxide (382 mg) added. The mixture was heated at 110° C. oil-bath temperature under closed atmosphere for 15 h, cooled and filtered through Celite®. The filtrate was evaporated and residue dissolved in 1,4-dioxane (17 ml) and again treated with selendioxide (200 mg). The mixture was then heated at 120° C. oil-bath temperature under closed atmosphere for 20 h, cooled and filtered through Celite®. The filtrate was evaporated and the residue dissolved in DMF (20 ml). After the addition of oxone (2.0 g), the mixture was stirred at room temperature for 17 h. Concentration and purification by chromatography (silica, CH₂Cl₂/MeOH) afforded the title compound (514 mg, 92%; MH⁺=201).

### PREPARATIVE EXAMPLE 13

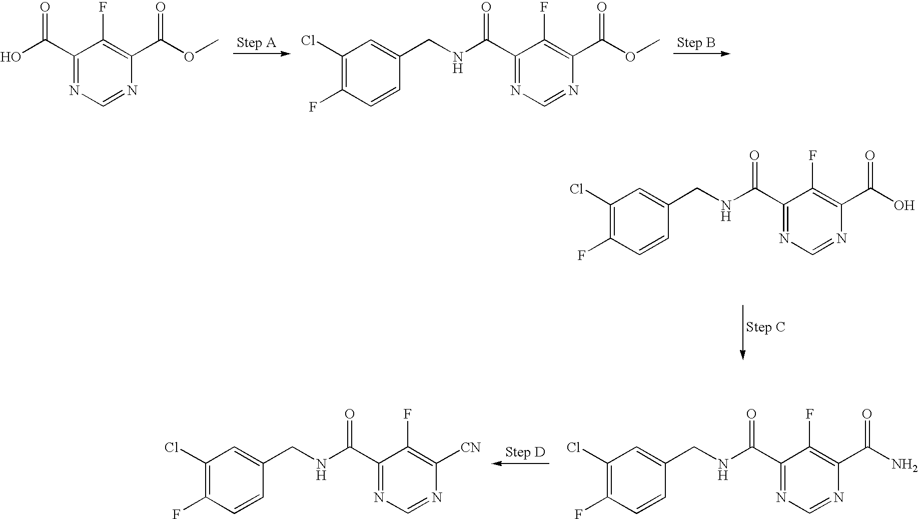

### Step A

To an ice cooled solution of the title compound from the Preparative Example 16, (360 mg) and commercially available 3-chloro-4-fluoro benzylamine (286 mg) in DMF (16 ml) were added N-methylmorpholine (272 μl), HATU (912 mg) and HOAt (326 mg). The mixture was stirred overnight while warming to room temperature and then concentrated. The remaining residue was dissolved in CHCl₃, washed with saturated aqueous NaHCO₃, 1N aqueous HCl and saturated aqueous NaCl, dried (MgSO₄), filtered, absorbed on silica and purified by chromatography (silica, CH₂Cl₂/MeOH) to afford the title compound title compound (195 mg, 32%; MH⁺=342).

### Step B

To a solution of NaOH (28 mg) in dry MeOH (3.7 ml) was added the title compound from Step A above (195 mg). The resulting suspension was stirred at room temperature for 1 h, acidified with 1N aqueous HCl and concentrated. The remaining residue was dissolved in EtOAc, washed with 1N aqueous HCl, dried (MgSO₄), filtered and concentrated to afford the title compound (175 mg, 93%; MH⁺=328).

### Step C

To a solution of the title compound from Step B above (175 mg) in anhydrous CH₂Cl₂ (2.7 mL) was added oxalyl chloride (0.17 ml) at 0° C., followed by the addition of anhydrous DMF (0.053 ml). The mixture was allowed to warm to room temperature, stirred for 1 h and concentrated. To the remaining reddish solid residue was added anhydrous CH₂Cl₂ (2.7 ml) at 0° C., followed by the addition of 0.5 M solution of NH₃ in 1,4-dioxane (1.1 ml). The mixture was allowed to warm to room temperature, stirred overnight, concentrated and purified by chromatography (silica, CH₂Cl₂/MeOH) to afford the title compound (160 mg, 92%; MH⁺=327).

### Step D

A 2 M solution of oxalyl chloride in CH₂Cl₂ (450 μl) was diluted in DMF (8 ml) and then cooled to 0° C. Pyridine (144 μl) and a solution of the title compound from Step C above (146 mg) in DMF (2 ml) were added and the mixture was stirred at 0° C. for 3 h and then at room temperature overnight. The mixture was concentrated, diluted with EtOAc, washed with saturated aqueous NaHCO₃, dried (MgSO₄), filtered and concentrated to afford the title compound (57 mg, 41%; MH⁺=309).

### PREPARATIVE EXAMPLE 14

Following a similar procedure as that described in Preparative Example 17 except using the amines in Step A as indicated in the table below, the following compounds were prepared.

| Preparative | | | |
|---|---|---|---|
| Example | Amine | Product | MH⁺ |
| 18 | 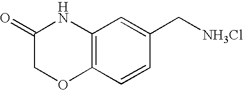 | 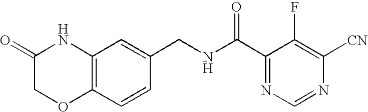 | 328 |

### PREPARATIVE EXAMPLE 15

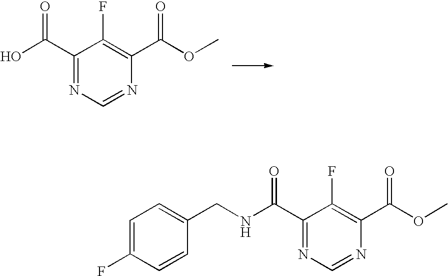

### Step A

To a stirring solution of the title compound from Preparative Example 16 above (˜450 mg) in DMF (10 ml) was added 4-fluorobenzylamine (320 mg), HATU (1000 mg) and HOAt (400 mg) followed by DIEA (750 μl). The mixture was stirred for 2 h. After concentration of the mixture to dryness the residue was diluted with ethyl acetate, washed with saturated sodium hydrogen carbonate and brine, dried (MgSO₄), concentrated to dryness and purified by silica chromatography (10% acetone in dichloromethane) to afford the title compound (300 mg, 29%; MH⁺=308).

### EXAMPLE 1

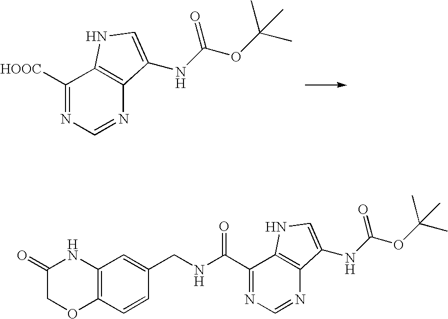

To the title compound from Preparative Example 3 (162 mg) were added EDCI (148 mg), HOAt (74 mg) and the title compound from Preparative Example 5 (130 mg). After the addition of DMF (5.6 ml) and DIEPA (94 μl) the mixture was stirred at room temperature overnight. After the solvents were removed in HV, the residue was dissolved in EtOAc (80 ml) and 10% citric acid solution (20 ml). The organic phase was separated, dried over MgSO₄, filtered and the solvents removed. The residue was purified by chromatography on silica using CH₂Cl₂/MeOH (95:5) as mobile phase to afford the title compound as a yellow solid (198 mg; 88%; MH⁺=439).

### EXAMPLE 2-3

Following a similar coupling procedure as that described in Example 1, except using the amines as indicated in the table below, the following compounds were prepared.

| | | | 1. |
|---|---|---|---|
| | | | Yield |
| | | | 2. |
| Example | Amine | Product | MH⁺ |
| 2 | 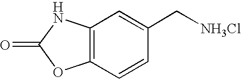 | 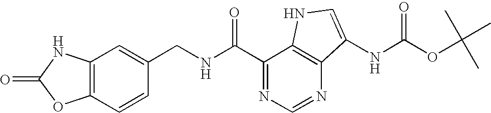 | 1.2. |
| 3 | 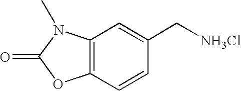 | 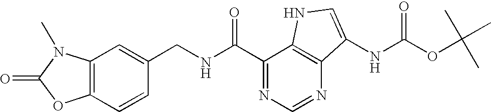 | 1.2. |

### EXAMPLE 4-8

Following a similar procedure as that described in Example 1, except using the compounds from the Preparative Examples and amines as indicated in the table below, the following compounds were prepared.

| Exam- | Preparative | | | 1. Yield |
|---|---|---|---|---|
| ple | Example | Amine | Product | 2. MH⁺ |
| 4 | 4 Step D | 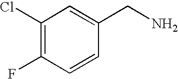 | 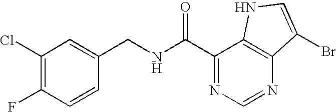 | 1. 63%2. 384/386 |
| 5 | 4 Step D | 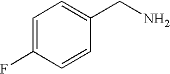 | 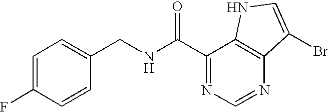 | 1. 69%2. 349/351 |
| 6 | 2 | 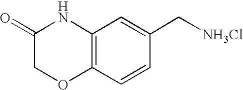 | 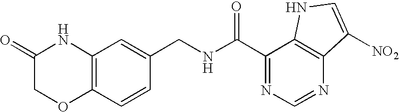 | 1. 57%2. 369 |
| 7 | 2 | 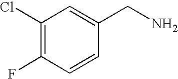 | 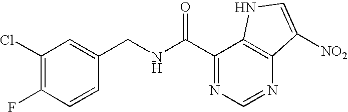 | 1. 64%2. 350/352 |
| 8 | 6 | 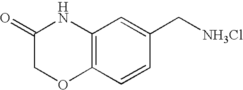 | 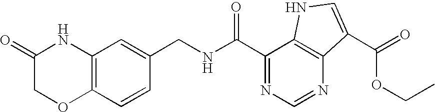 | 1. 73%2. 396 |

### EXAMPLE 9

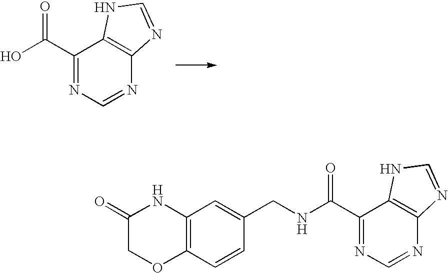

The title compound from Preparative Example 9 (52 mg), HATU (132 mg), HOAt (46 mg and the title compound from Preparative Example 5 (60 mg) were dissolved in DMF (5 ml) and DIEPA (110 μl) added. The mixture was stirred at room temperature overnight and then diluted with EtOAc/MeOH (95:5; 40 ml). The organic phase was washed with sat. NaHCO₃ (11 ml) and the aqueous layer extracted with EtOAc/MeOH (95:5; 40 ml. The combined organic phase was washed with brine, separated, dried over MgSO₄ and the solvents evaporated. The residue was further purified by chromatography on silica (CH₂Cl₂->CH₂Cl₂/MeOH (4:1) to afford the title compound as a colorless solid (36.2 mg; 43%; MH⁺=325).

### EXAMPLE 10-14

Following a similar coupling procedure as that described in Example 9, except using the compounds from the Preparative Examples and amines as indicated in the table below, the following compounds were prepared.

| | | | | 1. |
|---|---|---|---|---|
| | Compound | | | Yield |
| | Preparative | | | 2. |
| Example | Example | Amine | Product | MH⁺ |
| 10 | 10 | 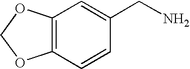 | 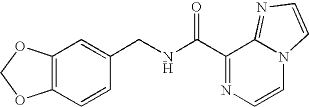 | 1. 11%2. 297 |
| 11 | 10 | 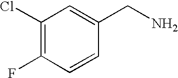 | 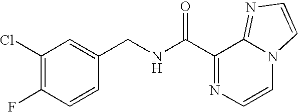 | 1. 18%2. 305 |
| 12 | 10 | 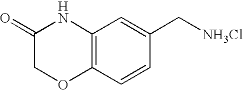 | 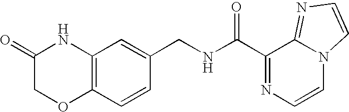 | 1. 34%2. 324 |
| 13 | 4 Step E | 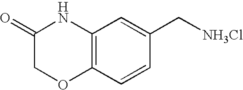 | 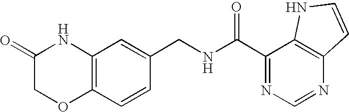 | 1. 24%2. 324 |
| 14 | 11 | 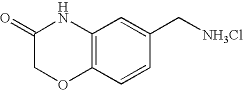 | 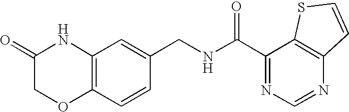 | 1. 81%2. 341 |

### EXAMPLE 15

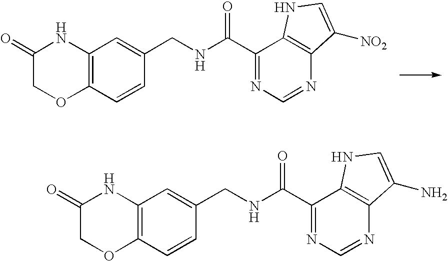

A mixture of the title compound from Example 6 (91 mg), AcOH (200 μl) and Pd/C (10 wt %, 55 mg) in THF/MeOH was hydrogenated at atmospheric pressure overnight, filtered, concentrated and diluted with saturated aqueous NaHCO₃. The formed precipitate was collected by filtration and purified by preparative thin layer chromatography (silica, CH₂Cl₂/MeOH) to afford the title compound as a brown solid (12 mg; 9%; MH⁺=339).

### EXAMPLE 16

Following a similar procedure as that described in Example 15, except using the compounds from the Examples as indicated in the table below, the following compounds were prepared.

| | | | 1. Yield |
|---|---|---|---|
| Example | Example | Product | 2. MH⁺ |
| 16 | 7 | 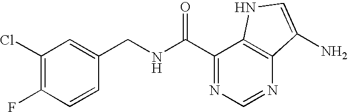 | 1. quant.2. 320/322 |

### EXAMPLE 17

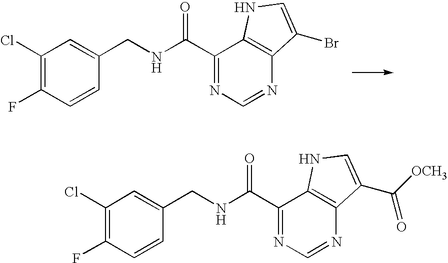

The title compound from Example 4 (50 mg) was dissolved in DMF (10 ml) and MeOH (10 ml) and TEA (60 μl) added. The mixture was sonicated for 10 Min while a stream of argon was bubbled through the solution. Then 1,1′-Bis-(diphenylphosphino)-ferrocen (8 mg) and Pd(OAc)₂ (4 mg) were added and the mixture carbonylated (7 bar CO) in a pressure reactor at 80° C. overnight. Since the reaction was not completed another batch of 1,1′-Bis-(diphenylphosphino)-ferrocen (8 mg) and Pd(OAc)₂ (4 mg) was added and the reaction continued for another 20 h at 100° C. After the addition of another batch of 1,1′-Bis-(diphenylphosphino)-ferrocen (8 mg) and Pd(OAc)₂ (4 mg), the reaction was continued 20 h at 115° C. The reaction mixture was then filtered and the filter washed with MeOH. The combined filtrate was evaporated, the residue dissolved/suspended in MeOH and silica added. The MeOH was evaporated and the coated silica loaded onto a silica column equilibrated with CH₂Cl₂. The column was then developed using a gradient (CH₂Cl₂->CH₂Cl₂/MeOH (99:1). Fractions containing the product were collected and the solvents evaporated to afford the title compound as off white solid (29.7 mg; 63%; MH⁺=363/365).

### EXAMPLE 18

Following a similar procedure as that described in Example 17, except using the compounds from the Examples indicated in the table below, the following compounds were prepared.

| | | | 1. Yield |
|---|---|---|---|
| Example | Example | Product | 2. MH⁺ |
| 18 | 5 | 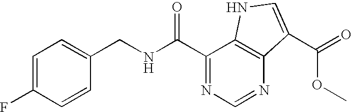 | 1. 74%2. 329 |

### EXAMPLE 19

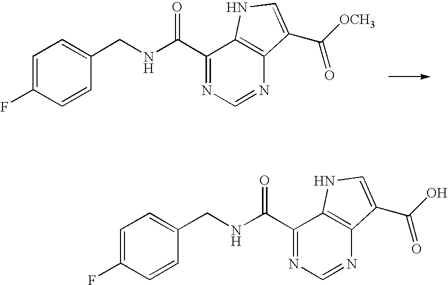

The title compound from Example 15 (269 mg) was suspended in THF (20 ml), 1,4-dioxane (15 ml) and H₂O (20 ml). After the addition of LiOH×H₂O (342 mg) the mixture was heated at 70° C. for 90 Min. Another batch of LiOH×H₂O (342 mg) was added and heating at 70° C. was continued for 20 h. The mixture concentrated, acidified to pH˜1.5 by adding 1 M HCl and then extracted with EtOAc (3×20 ml). The combined organic phase was washed with brine, separated, dried over MgSO₄, filtered and the solvents evaporated to afford the title compound as off white solid (195.7 mg; 76%; MH⁺=315).

### EXAMPLE 20

Following a similar procedure as that described in Example 19, except using the compounds from the Examples indicated in the table below, the following compounds were prepared.

| | | | 1. Yield |
|---|---|---|---|
| Example | Example | Product | 2. MH⁺ |
| 20 | 17 | 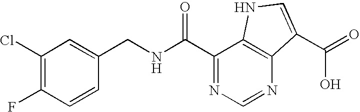 | 1. 77%2. 349/351 |

### EXAMPLE 21

Following a similar procedure as that described in Example 19, except using the compounds from the Examples as indicated in the table below, the following compounds were prepared.

| | | | 1. Yield |
|---|---|---|---|
| Example | Example | Product | 2. MH⁺ |
| 21 | 8 | 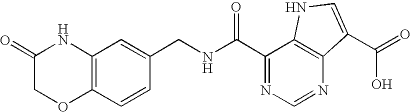 | 1. 78%2. 368 |

### EXAMPLE 22

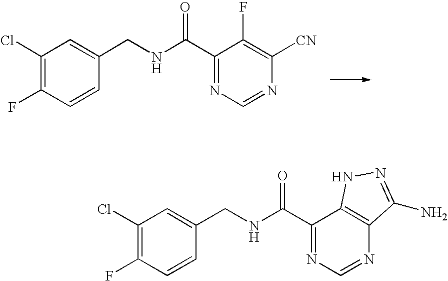

To a stirring solution of the title compound from Preparative Example 13 (9 mg) in 1,4-dioxane (3 ml) was added a 1 M solution of hydrazine hydrate in 1,4-dioxane (45 μl). The mixture was stirred at room temperature for 3 h and then concentrated to afford the title compound (10 mg, >99%; MH⁺=321).

### EXAMPLE 23

Following a similar procedure as that described in Example 22 except using the compounds from the Preparative Examples as indicated in the table below, the following compounds were prepared.

| | | | 1. Yield |
|---|---|---|---|
| Example | Preparative Example | Product | 2. MH⁺ |
| 23 | 14 | 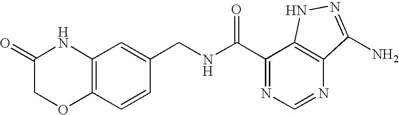 | 1. >99%2. 340 |

### EXAMPLE 24

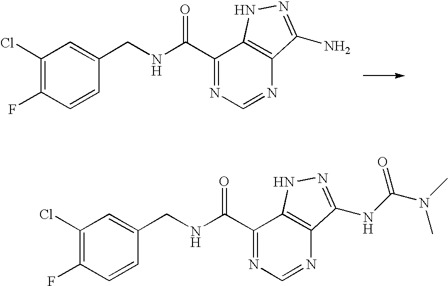

The title compound from Example 22 (41 mg) was dissolved pyridine (1 ml) and commercially available dimethylcarbamoylchloride (13 μl) added. The mixture was stirred at room temperature for 3 days. Then another batch of dimethylcarbamoylchloride (100 μl) was added, and stirring at room temperature was continued for another 3 days. The mixture was evaporated and the residue purified by PREP-TLC using CH₂Cl₂/MeOH (9:1) as mobile phase to afford the title compound (18 mg; 36%; MH⁺=392/394).

### EXAMPLE 25

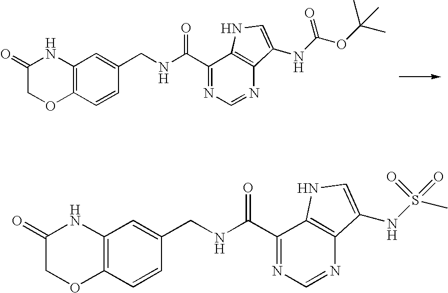

The title compound from Example 1 (10.5 mg) was treated with a solution of 4 M HCl in dioxane (700 μl) and the mixture stirred at room temperature for 3 h. The solvents were evaporated and the residue suspended in THF (0.5 ml) and TEA (0.18 ml). The mixture was treated with methanesulfonyl chloride (3 μl) and the mixture was stirred at room temperature overnight and the solvents removed. The residue was treated with 10% citric acid solution (5 ml), sonicated for 1 Min and allowed to stand at room temperature for 30 Min. The precipitate was collected by filtration, washed with H₂O (5 ml) and then dried in HV. to afford the title compound as a grey solid (4 mg; 40%; MH⁺=417).

### EXAMPLE 26-28

Following a similar procedure as that described in Example 25, except using the compounds from the examples and the sulfonyl chlorides or acid chlorides as indicated in the table below, the following compounds were prepared.

| | Sulfonyl | | |
|---|---|---|---|
| | chloride/ | | |
| | isocyanates/ | | 1. Yield |
| Example | esters | Product | 2. MH⁺ |
| 26 | 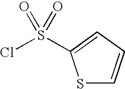 | 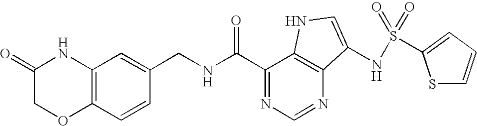 | 1. 23%2. 485 |
| 27 | 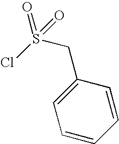 | 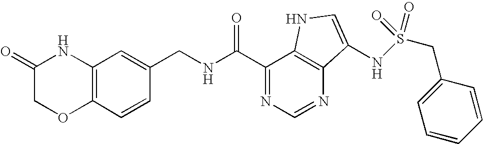 | 1. 24%2. 493 |
| 28 | 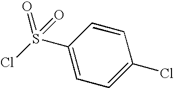 | 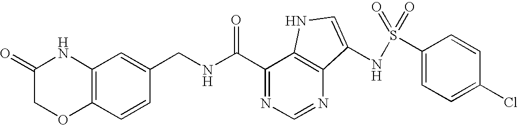 | 1. 27%2. 513 |

### EXAMPLE 29

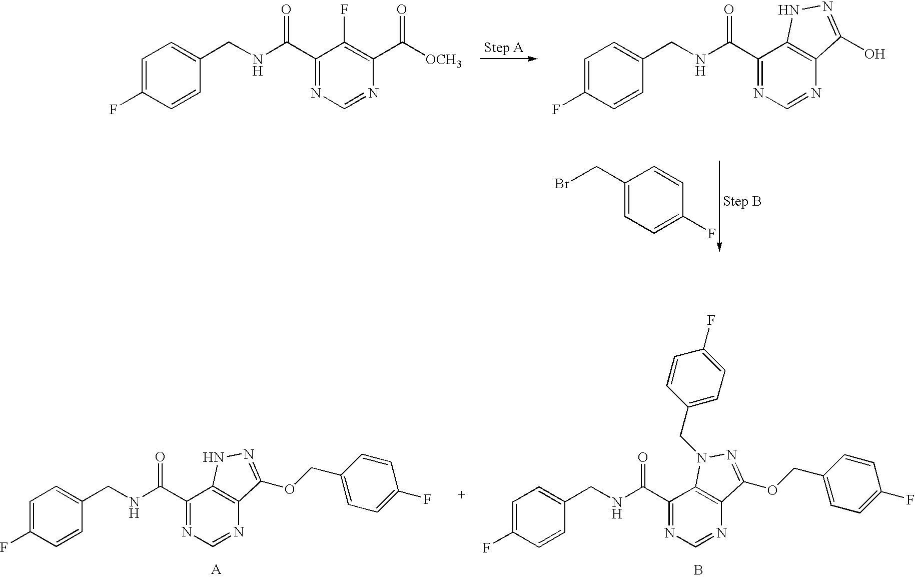

### Step A

To a stirring solution of the title compound from Preparative Example 15 (3.4 mg) in 1,4-dioxane (500 μL) was added a 1 M solution of hydrazine hydrate in 1,4-dioxane (10 μL) and the mixture was stirred for 3 h. The solvents were evaporated to afford the title compound (3.2 mg; 99%; MH⁺=288).

### Step B

To a stirring solution of the title compound from Step A above (11.5 mg) and K₂CO₃ in DMF (3 mL) was added drop wise at 50° C. a solution of commercially available 1-(bromomethyl)-4-fluorobenzene (10 μL) in DMF (490 μL) and the mixture was stirred for 2 h at 50° C. After concentration of the mixture to dryness the residue purified by silica chromatography (5% acetone in dichloromethane) to afford the two title compounds A (1.2 mg. 8%; MH⁺=396) and B (4.2 mg. 21%, MH⁺=504).

### EXAMPLE 30

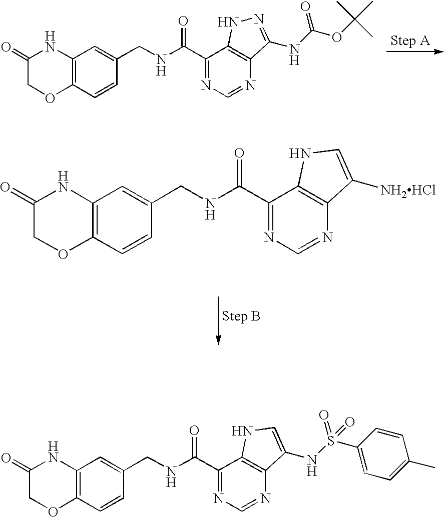

### Step A

To a suspension of the title compound from the Example 1 (939 mg) in EtOAc (17.1 ml) was added a 4M solution of HCl in 1,4-dioxane (17.1 ml). The reaction mixture was stirred at room temperature for 20 h and concentrated to afford the title compound (850 mg; >99%; [M-Cl]⁺=339)

### Step B

To a suspension of the title compound from the Step A above (2.8 mg) in dry pyridine (75 μl) was added a 0.1 M solution of commercially available 4-Methyl-benzenesulfonyl chloride in 1,2-dichlorethane (75 μl). The resulting mixture was agitated (˜800 rpm) at room temperature for 15 h, concentrated and dried in vacuo for 12 h to afford the crude title compound. [MH]⁺=449.

### EXAMPLES 31-69

Following a similar procedure as described in the Example 30, except using the sulfonyl chlorides indicated below, the following compounds were prepared.

| Ex. # | Sulfonyl chloride, Amine | Product | Yield |
|---|---|---|---|
| 31 | 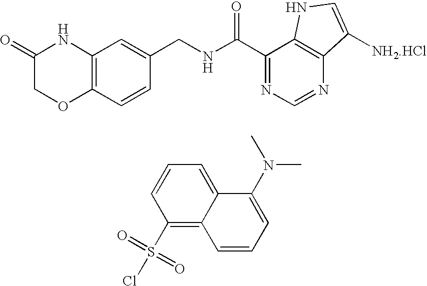 | 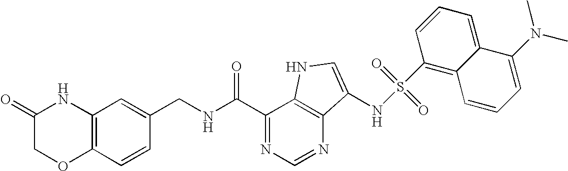 | n.d.[MH]⁺ = 572 |
| 32 | 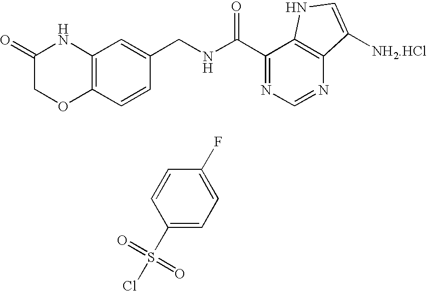 | 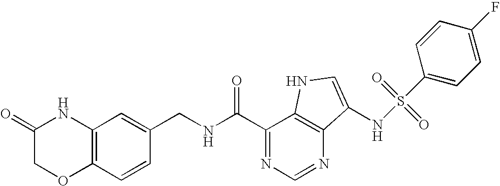 | n.d.[MH]⁺ = 497 |
| 33 | 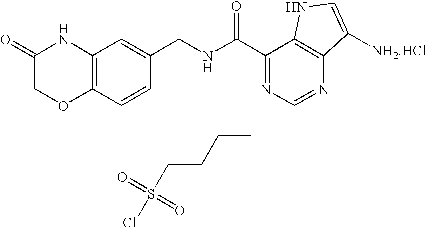 | 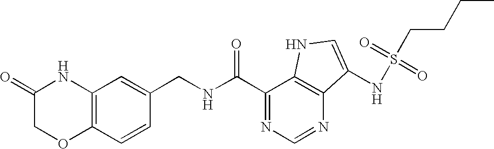 | n.d.[MH]⁺ = 459 |
| 34 | 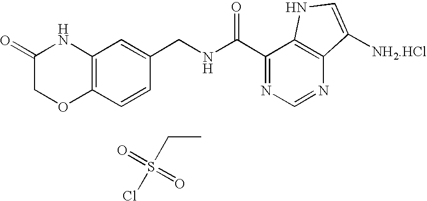 | 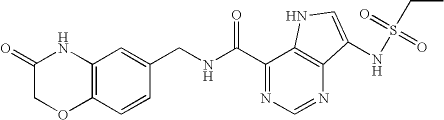 | n.d.[MH]⁺ = 431 |
| 35 | 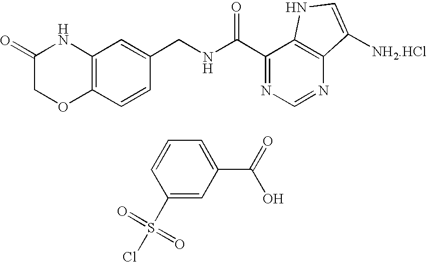 | 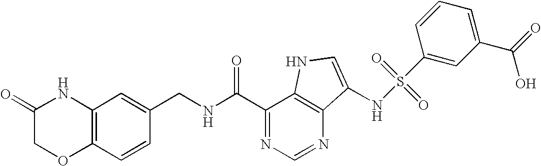 | n.d.[MH]⁺ = 523 |
| 36 | 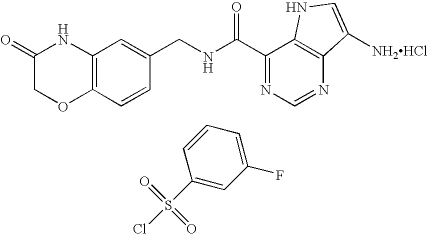 | 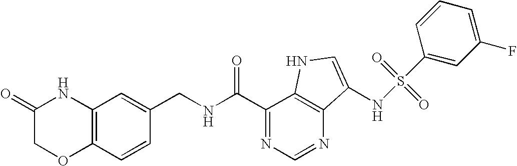 | n.d.[MH]⁺ = 497 |
| 37 | 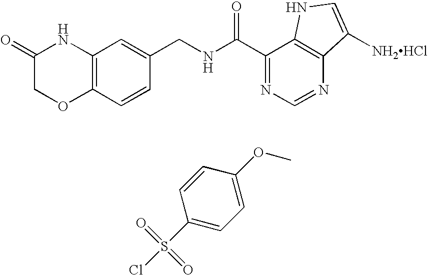 | 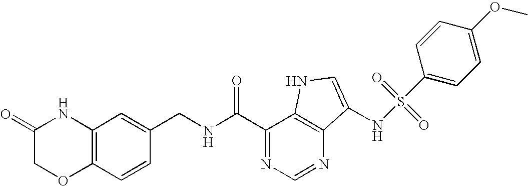 | n.d.[MH]⁺ = 509 |
| 38 | 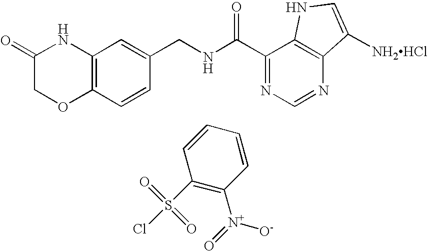 | 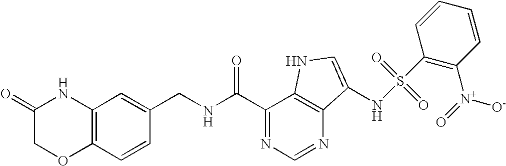 | n.d.[MH]⁺ = 524 |
| 39 | 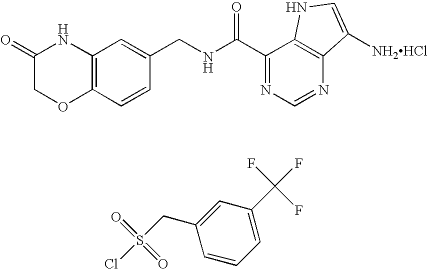 | 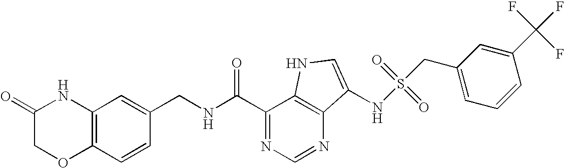 | n.d.[MH]⁺ = 561 |
| 40 | 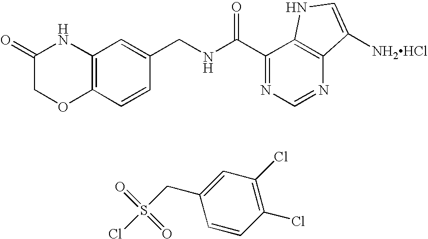 | 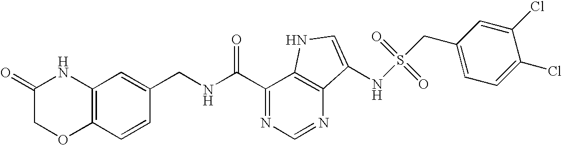 | n.d.[MH]⁺ = 562 |
| 41 | 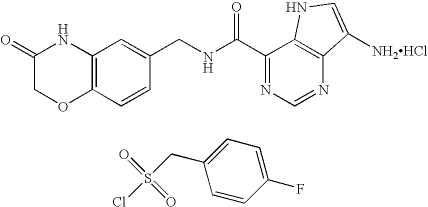 | 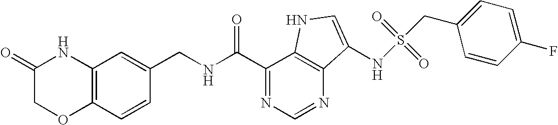 | n.d.[MH]⁺ = 511 |
| 42 | 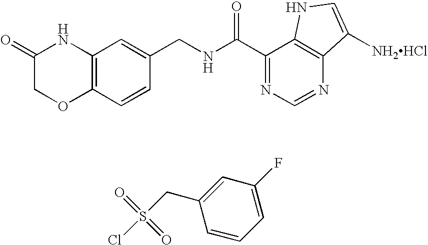 | 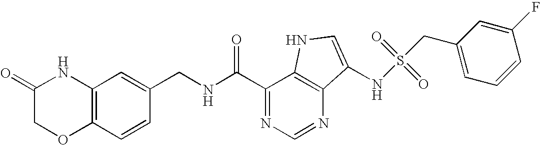 | n.d.[MH]⁺ = 511 |
| 43 | 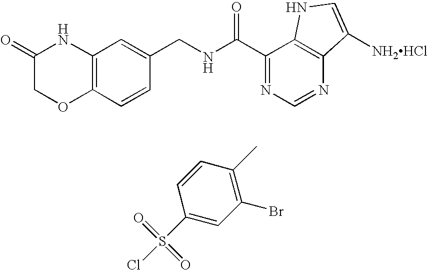 | 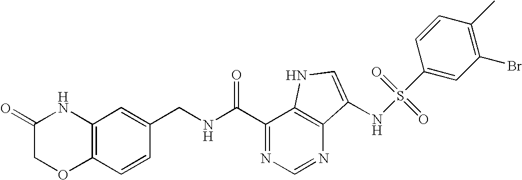 | n.d.[MH]⁺ = 572 |
| 44 | 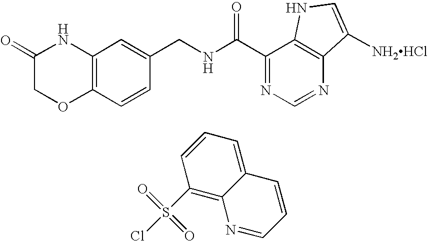 | 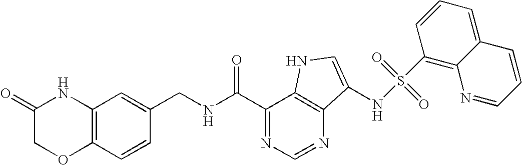 | n.d.[MH]⁺ = 530 |
| 45 | 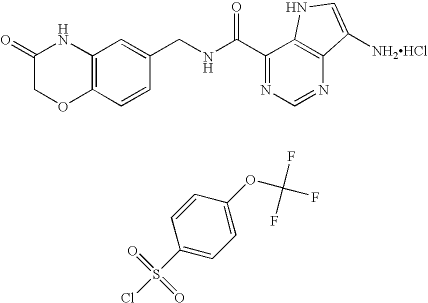 | 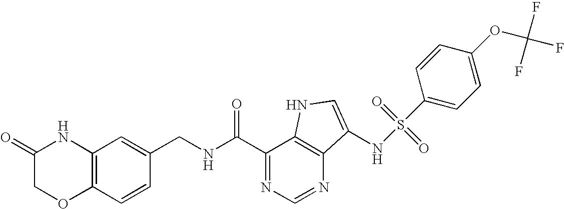 | n.d.[MH]⁺ = 563 |
| 46 | 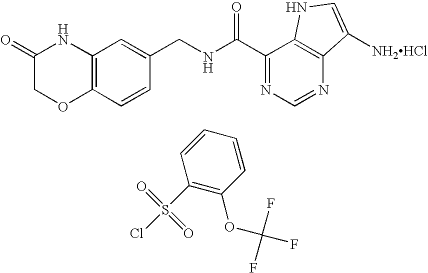 | 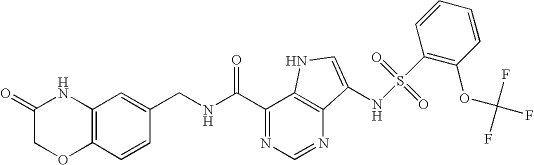 | n.d.[MH]⁺ = 563 |
| 47 | 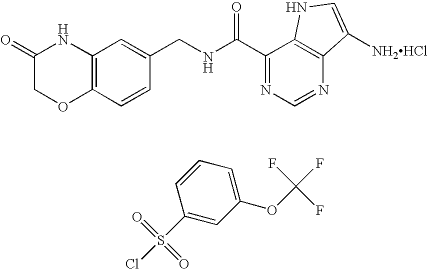 | 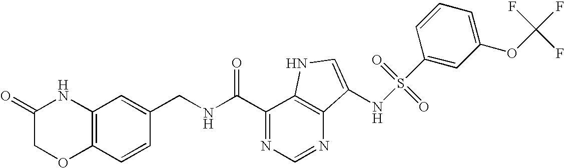 | n.d.[MH]⁺ = 563 |
| 48 | 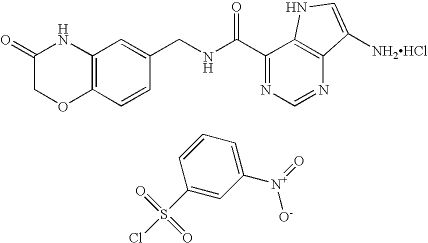 | 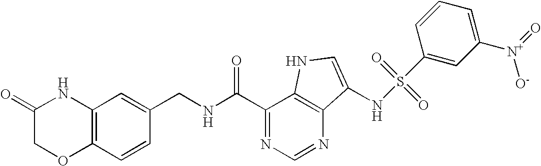 | n.d.[MH]⁺ = 524 |
| 49 | 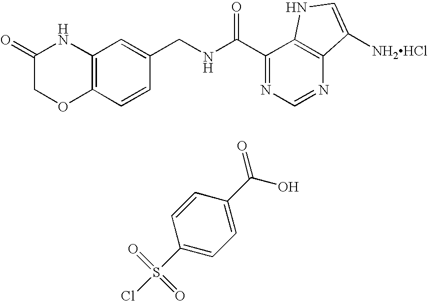 | 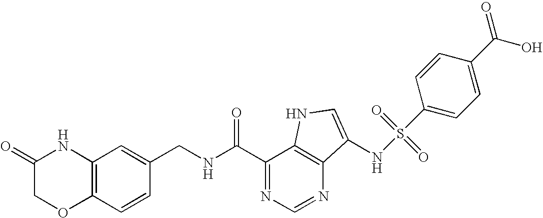 | n.d.[MH]⁺ = 523 |
| 50 | 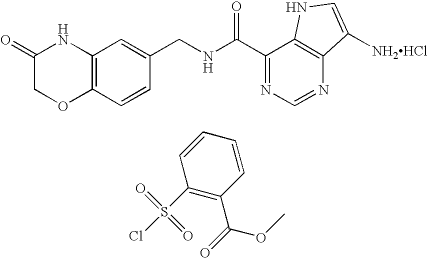 | 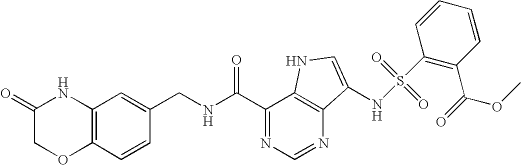 | n.d.[MH]⁺ = 537 |
| 51 | 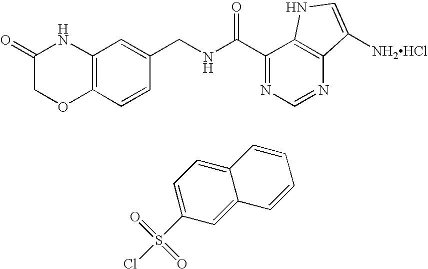 | 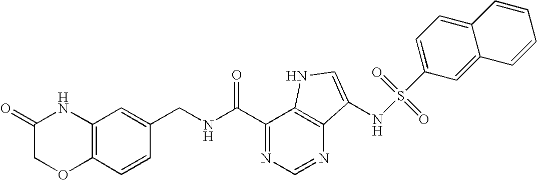 | n.d.[MH]⁺ = 529 |
| 52 | 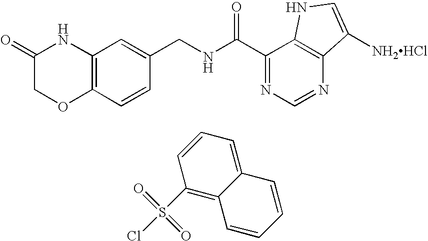 | 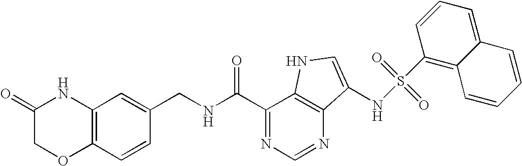 | n.d.[MH]⁺ = 529 |
| 53 | 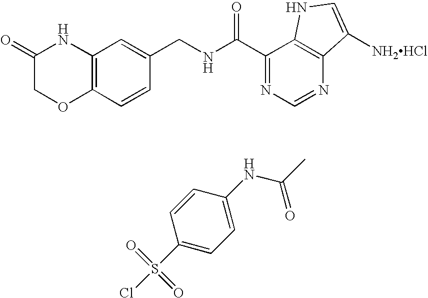 | 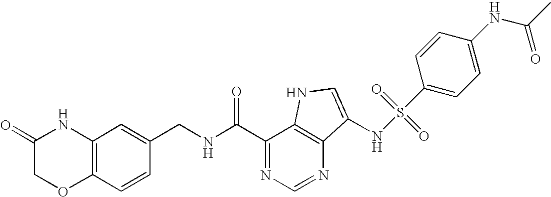 | n.d.[MH]⁺ = 536 |
| 54 | 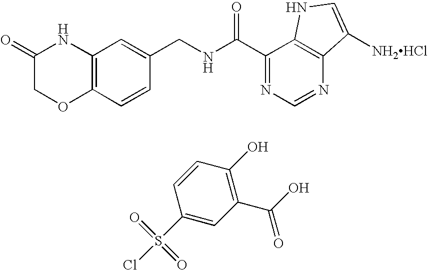 | 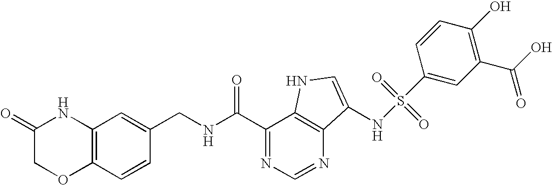 | n.d.[MH]⁺ = 539 |
| 55 | 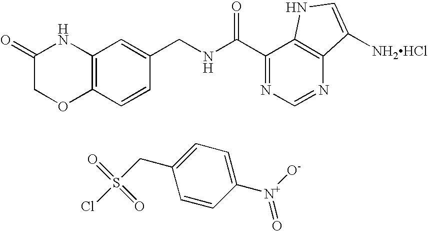 | 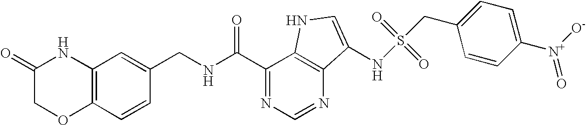 | n.d.[MH]⁺ = 538 |
| 56 | 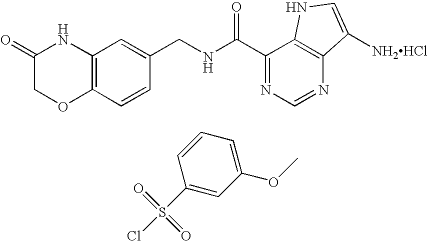 | 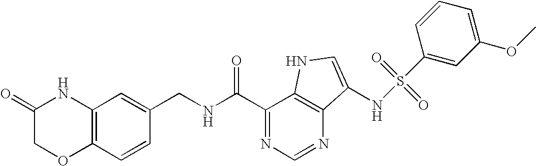 | n.d.[MH]⁺ = 509 |
| 57 | 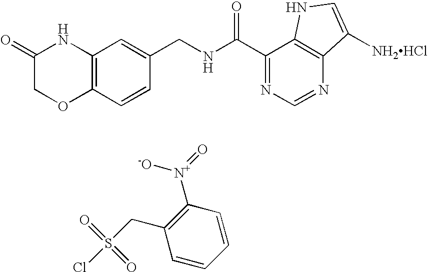 | 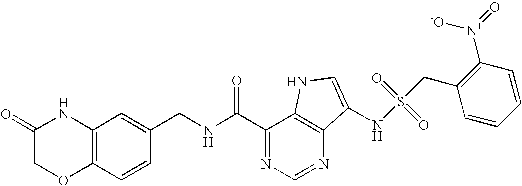 | n.d.[MH]⁺ = 538 |
| 58 | 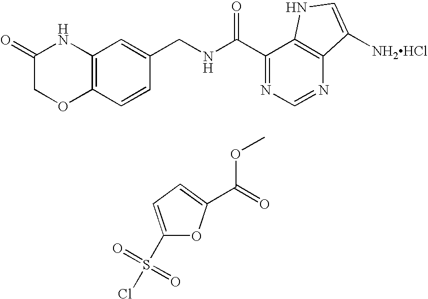 | 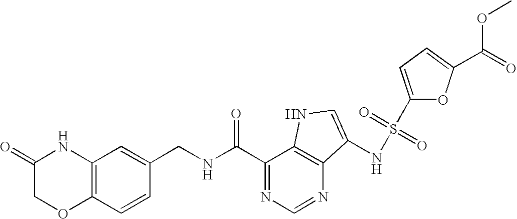 | n.d.[MH]⁺ = 527 |
| 59 | 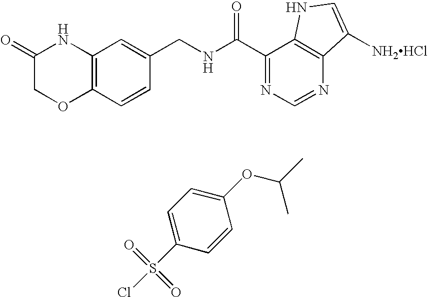 | 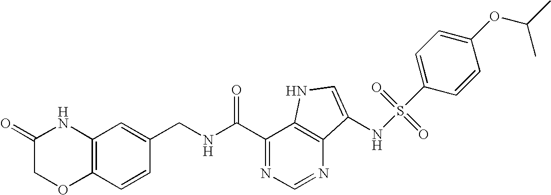 | n.d.[MH]⁺ = 537 |
| 60 | 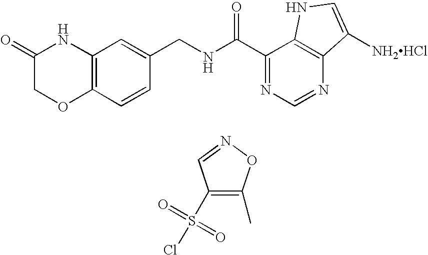 | 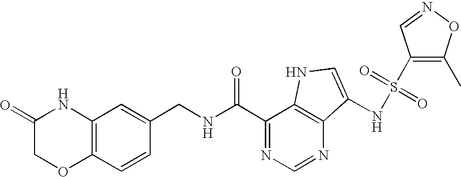 | n.d.[MH]⁺ = 484 |
| 61 | 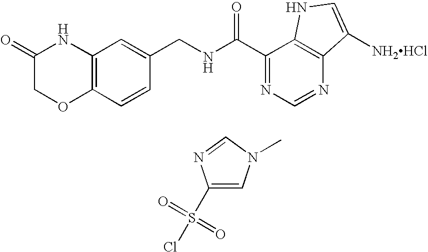 | 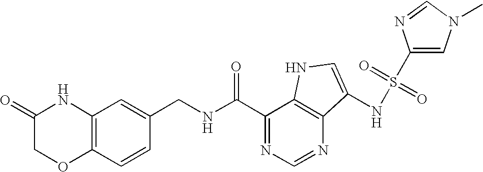 | n.d.[MH]⁺ = 483 |
| 62 | 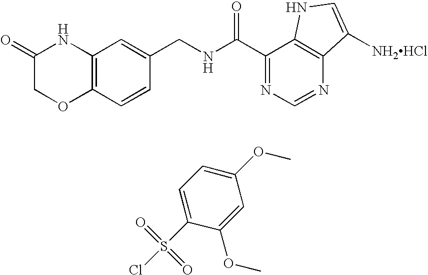 | 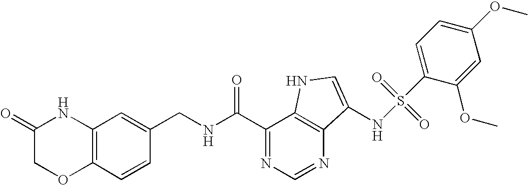 | n.d.[MH]⁺ = 539 |
| 63 | 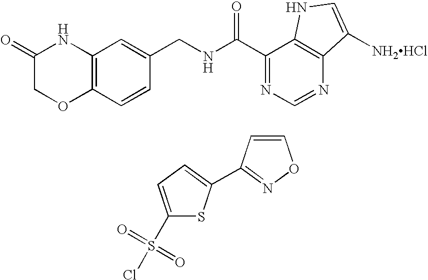 | 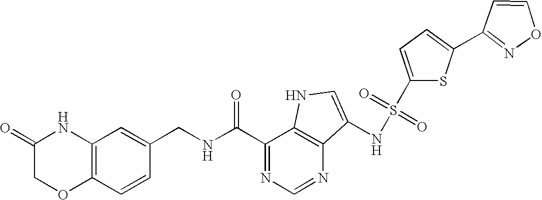 | n.d.[MH]⁺ = 552 |
| 64 | 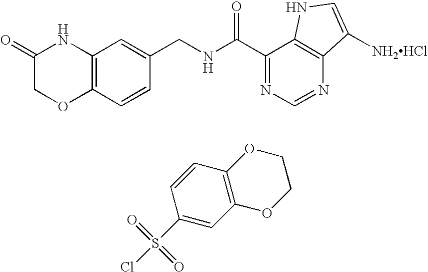 | 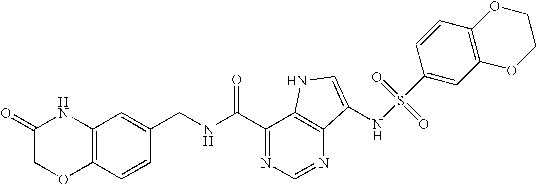 | n.d.[MH]⁺ = 537 |
| 65 | 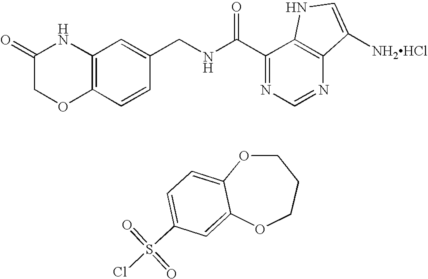 | 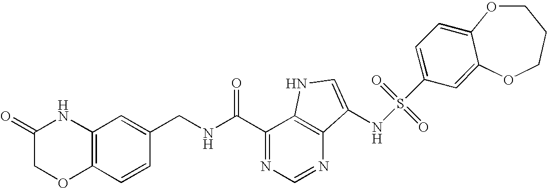 | n.d.[MH]⁺ = 551 |
| 66 | 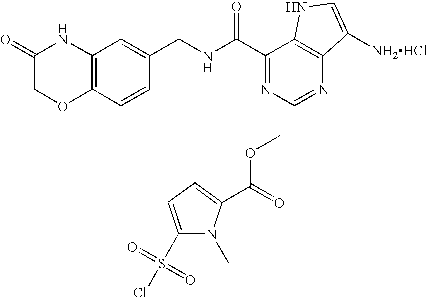 | 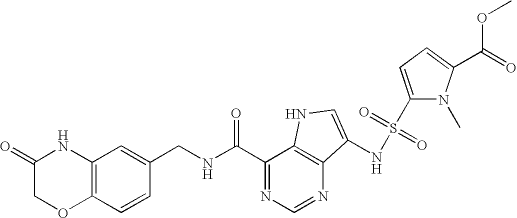 | n.d.[MH]⁺ = 540 |
| 67 | 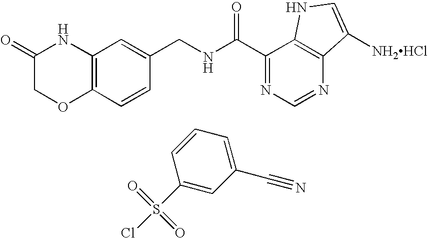 | 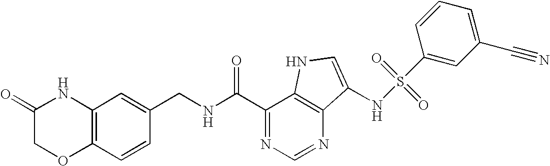 | n.d.[MH]⁺ = 504 |
| 68 | 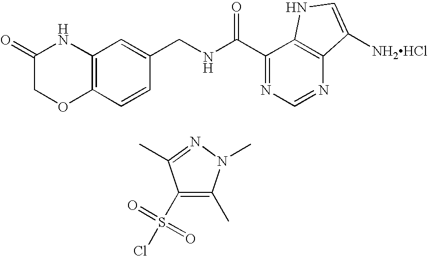 | 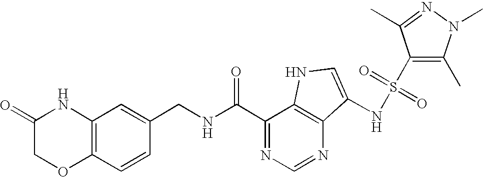 | n.d.[MH]⁺ = 511 |
| 69 | 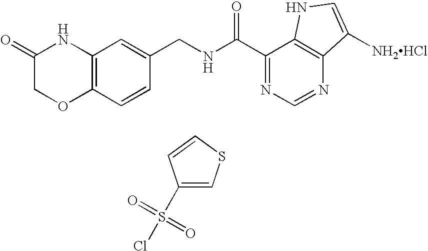 | 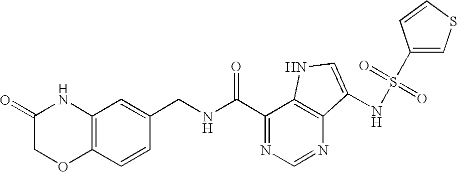 | n.d.[MH]⁺ = 485 |

n.d. (not determined)

### EXAMPLE 1700

### Assay for Determining MMP-13 Inhibition

The typical assay for MMP-13 activity is carried out in assay buffer comprised of 50 mM Tris, pH 7.5, 150 mM NaCl, 5 mM CaCl₂ and 0.05% Brij-35. Different concentrations of tested compounds are prepared in assay buffer in 50 μl aliquots. 10 μl of a 50 nM stock solution of catalytic domain of MMP-13 enzyme (produced by Alantos or commercially available from Invitek (Berlin), Cat. No. 30100812) is added to the compound solution. The mixture of enzyme and compound in assay buffer is thoroughly mixed and incubated for 10 min at room temperature. Upon the completion of incubation, the assay is started by addition of 40 μl of a 12.5 μM stock solution of MMP-13 fluorescent substrate (Calbiochem, Cat. No. 444235). The time-dependent increase in fluorescence is measured at the 320 nm excitation and 390 nm emission by automatic plate multireader. The IC₅₀ values are calculated from the initial reaction rates.

### EXAMPLE 1701

### Assay for Determining MMP-3 Inhibition

The typical assay for MMP-3 activity is carried out in assay buffer comprised of 50 mM MES, pH 6.0, 10 mM CaCl₂ and 0.05% Brij-35. Different concentrations of tested compounds are prepared in assay buffer in 50 μl aliquots. 10 μl of a 100 nM stock solution of the catalytic domain of MMP-3 enzyme (Biomol, Cat. No. SE-109) is added to the compound solution. The mixture of enzyme and compound in assay buffer is thoroughly mixed and incubated for 10 min at room temperature. Upon the completion of incubation, the assay is started by addition of 40 μl of a 12.5 μM stock solution of NFF-3 fluorescent substrate (Calbiochem, Cat. No. 480455). The time-dependent increase in fluorescence is measured at the 330 nm excitation and 390 nm emission by an automatic plate multireader. The IC₅₀ values are calculated from the initial reaction rates.

### EXAMPLE 1702

### Assay for Determining MMP-8 Inhibition

The typical assay for MMP-8 activity is carried out in assay buffer comprised of 50 mM Tris, pH 7.5, 150 mM NaCl, 5 mM CaCl₂ and 0.05% Brij-35. Different concentrations of tested compounds are prepared in assay buffer in 50 μl aliquots. 10 μl of a 50 nM stock solution of activated MMP-8 enzyme (Calbiochem, Cat. No. 444229) is added to the compound solution. The mixture of enzyme and compound in assay buffer is thoroughly mixed and incubated for 10 min at 37° C. Upon the completion of incubation, the assay is started by addition of 40 μl of a 10 μM stock solution of OmniMMP fluorescent substrate (Biomol, Cat. No. P-126). The time-dependent increase in fluorescence is measured at the 320 nm excitation and 390 nm emission by an automatic plate multireader at 37° C. The IC₅₀ values are calculated from the initial reaction rates.

### EXAMPLE 1703

### Assay for Determining MMP-12 Inhibition

The typical assay for MMP-12 activity is carried out in assay buffer comprised of 50 mM Tris, pH 7.5, 150 mM NaCl, 5 mM CaCl₂ and 0.05% Brij-35. Different concentrations of tested compounds are prepared in assay buffer in 50 μl aliquots. 10 μl of a 50 nM stock solution of the catalytic domain of MMP-12 enzyme (Biomol, Cat. No. SE-138) is added to the compound solution. The mixture of enzyme and compound in assay buffer is thoroughly mixed and incubated for 10 min at room temperature. Upon the completion of incubation, the assay is started by addition of 401 of a 12.5 μM stock solution of OmniMMP fluorescent substrate (Biomol, Cat. No. P-126). The time-dependent increase in fluorescence is measured at the 320 nm excitation and 390 nm emission by automatic plate multireader at 37° C. The IC₅₀ values are calculated from the initial reaction rates.

### EXAMPLE 1704

### Assay for Determining Aggrecanase-1 Inhibition

The typical assay for aggrecanase-1 activity is carried out in assay buffer comprised of 50 mM Tris, pH 7.5, 150 mM NaCl, 5 mM CaCl₂ and 0.05% Brij-35. Different concentrations of tested compounds are prepared in assay buffer in 50 μl aliquots. 10 μl of a 75 nM stock solution of aggrecanase-1 (Invitek) is added to the compound solution. The mixture of enzyme and compound in assay buffer is thoroughly mixed. The reaction is started by addition of 40 μl of a 250 nM stock solution of aggrecan-IGD substrate (Invitek) and incubation at 37° C. for exact 15 min. The reaction is stopped by addition of EDTA and the samples are analysed by using aggrecanase ELISA (Invitek, InviLISA, Cat. No. 30510111) according to the protocol of the supplier. Shortly: 100 μl of each proteolytic reaction are incubated in a pre-coated micro plate for 90 min at room temperature. After 3 times washing, antibody-peroxidase conjugate is added for 90 min at room temperature. After 5 times washing, the plate is incubated with TMB solution for 3 min at room temperature. The peroxidase reaction is stopped with sulfurous acid and the absorbance is red at 450 nm. The IC₅₀ values are calculated from the absorbance signal corresponding to residual aggrecanase activity.

### EXAMPLE 1705

### Assay for Determining Inhibition of MMP-3 Mediated Proteoglycan Degradation

The assay for MMP-3 activity is carried out in assay buffer comprised of 50 mM MES, pH 6.0, 10 mM CaCl₂ and 0.05% Brij-35. Articular cartilage is isolated fresh from the first phalanges of adult cows and cut into pieces (˜3 mg). Bovine cartilage is incubated with 50 nM human MMP-3 (Chemikon, cat.#25020461) in presence or absence of inhibitor for 24 h at 37° C. Sulfated glycosaminoglycan (aggrecan) degradation products (sGAG) are detected in supernatant, using a modification of the colorimetric DMMB (1,9-dimethylmethylene blue dye) assay (Billinghurst et al., 2000, Arthritis & Rheumatism, 43 (3), 664). 10 μl of the samples or standard are added to 190 μl of the dye reagent in microtiter plate wells, and the absorbance is measured at 525 nm immediately. All data points are performed in triplicates.

### EXAMPLE 1706

### Assay for Determining Inhibition of MMP-3 Mediated Pro-Collagenase 3 Activation

The assay for MMP-3 mediated activation of pro-collagenase 3 (pro-MMP-13) is carried out in assay buffer comprised of 50 mM MES, pH 6.0, 10 mM CaCl2 and 0.05% Brij-35 (Nagase; J. Biol. Chem. 1994 Aug. 19; 269(33):20952-7).

Different concentrations of tested compounds are prepared in assay buffer in 5 μL aliquots. 10 mL of a 100 nM stock solution of trypsin-activated (Knäuper V., et al., 1996 J. Biol. Chem. 271 1544-1550) human pro-MMP-3 (Chemicon; CC1035) is added to the compound solution. To this mixture, 35 μl of a 286 nM stock solution of pro-collagenase 3 (Invitek; 30100803) is added to the mixture of enzyme and compound. The mixture is thoroughly mixed and incubated for 5 h at 37° C. Upon the completion of incubation, 10 μl of the incubation mixture is added to 50 μL assay buffer comprised of 50 mM Tris, pH 7.5, 150 mM NaCl, 5 mM CaCl2 and 0.05% Brij-35 and the mixture is thoroughly mixed.

The assay to determine the MMP-13 activity is started by addition of 40 μL of a 10 μM stock solution of MMP-13 fluorogenic substrate (Calbiochem, Cat. No. 444235) in assay buffer comprised of 50 mM Tris, pH 7.5, 150 mM NaCl, 5 mM CaCl2 and 0.05% Brij-35 (Knäuper, V., et al., 1996. J. Biol. Chem. 271, 1544-1550). The time-dependent increase in fluorescence is measured at 320 nm excitation and 390 nm emission by an automatic plate multireader at room temperature. The IC50 values are calculated from the initial reaction rates.

We claim:

1. A compound having Formula (I): 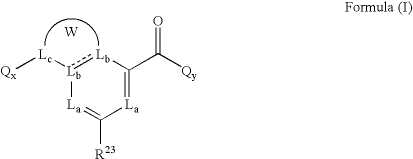
wherein:
R¹ in each occurrence is independently selected from hydrogen, alkyl, haloalkyl, trifluoroalkyl, alkenyl, alkynyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl,
wherein R¹ is optionally substituted one or more times, or
wherein R¹ is optionally substituted by one R¹⁶ group and optionally substituted by one or more R⁶ groups;
R² in each occurrence is selected from hydrogen and alkyl, wherein alkyl is optionally substituted one or more times or R¹ and R² when taken together with the nitrogen to which they are attached complete a 3- to 8-membered ring containing carbon atoms and optionally containing a heteroatom selected from O, S(O)ₓ, or NR⁵⁰ and which is optionally substituted one or more times;
R⁴ in each occurrence is independently selected from R¹⁰, hydrogen, alkyl, cycloalkyl, heterocycloalkyl, aryl, heteroaryl, halo, haloalkyl, CF₃, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, (C₀-C₆)-alkyl-C(O)—NR¹¹—CN, O—(C₀-C₆)-alkyl-C(O)NR¹⁰R¹¹, S(O)ₓ—(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹⁰, O—(C₀-C₆)-alkyl-aryl and O—(C₀-C₆)-alkyl-heteroaryl,
wherein each R⁴ group is optionally substituted one or more times, or
wherein each R⁴ group is optionally substituted by one or more R¹⁴ groups;
R⁵ in each occurrence is independently selected from hydrogen, alkyl, C(O)NR¹⁰R¹¹, aryl, arylalkyl, SO₂NR¹⁰R¹¹ and C(O)OR¹⁰, wherein alkyl, aryl and arylalkyl are optionally substituted one or more times;
R⁶ is independently selected from R⁹, alkenyl, alkynyl, cycloalkyl, heterocycloalkyl, bicycloalkyl, heterobicycloalkyl, spiroalkyl, spiroheteroalkyl, aryl, heteroaryl, C(O)OR¹⁰, CH(CH₃)CO₂H, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-P(O)₂OH, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-NR¹⁰C(═N—CN)NR¹⁰R¹¹, (C₀-C₆)-alkyl-C(═N—CN)NR¹⁰R¹¹, (C₀-C₆)-alkyl-NR¹⁰C(═N—NO₂)NR¹⁰R¹¹, (C₀-C₆)-alkyl-C(═N—NO₂)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹⁰, C(O)NR¹⁰—(C₀-C₆)-alkyl-heteroaryl, C(O)NR¹⁰—(C₀-C₆)-alkyl-aryl, S(O)₂NR¹⁰—(C₀-C₆)-alkyl-aryl, S(O)₂NR¹⁰—(C₀-C₆)-alkyl-heteroaryl, S(O)₂NR¹⁰-alkyl, S(O)₂—(C₀-C₆)-alkyl-aryl, S(O)₂—(C₀-C₆)-alkyl-heteroaryl, (C₀-C₆)-alkyl-C(O)—NR¹¹—CN, O—(C₀-C₆)-alkyl-C(O)NR¹⁰R¹¹, S(O)ₓ—(C₀-C₆)-alkyl-C(O)OR¹⁰, S(O), —(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹¹, O—(C₀-C₆)-alkyl-aryl and O—(C₀-C₆)-alkyl-heteroaryl,
wherein each R⁶ group is optionally substituted one or more times, or
wherein each R⁶ group is optionally substituted by one or more R¹⁴ groups;
R⁹ in each occurrence is independently selected from R¹⁰, hydrogen, alkyl, cycloalkyl, heterocycloalkyl, aryl, heteroaryl, halo, CHF₂, CF₃, OR¹⁰, SR¹⁰, COOR¹⁰, CH(CH₃)CO₂H, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-P(O)₂OH, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-NR¹⁰C(═N—CN)NR¹⁰R¹¹, (C₀-C₆)-alkyl-C(═N—CN)NR¹⁰R¹¹, (C₀-C₆)-alkyl-NR¹⁰C(═N—NO₂)NR¹⁰R¹¹, (C₀-C₆)-alkyl-C(═N—NO₂)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, C(O)NR¹⁰—(C₀-C₆)-alkyl-heteroaryl, C(O)NR¹⁰—(C₀-C₆)-alkyl-aryl, S(O)₂NR¹⁰—(C₀-C₆)-alkyl-aryl, S(O)₂NR¹⁰—(C₀-C₆)-alkyl-heteroaryl, S(O)₂NR¹⁰-alkyl, S(O)₂—(C₀-C₆)-alkyl-aryl, S(O)₂—(C₀-C₆)-alkyl-heteroaryl, (C₀-C₆)-alkyl-C(O)—NR¹¹—CN, O—(C₀-C₆)-alkyl-C(O)NR¹⁰R¹¹, S(O)ₓ—(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹¹, O—(C₀-C₆)-alkyl-aryl and O—(C₀-C₆)-alkyl-heteroaryl,
wherein each R⁹ group is optionally substituted, or
wherein each R⁹ group is optionally substituted by one or more R¹⁴ groups;
R¹⁰ and R¹¹ in each occurrence are independently selected from hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted, or R¹⁰ and R¹¹ when taken together with the nitrogen to which they are attached complete a 3- to 8-membered ring containing carbon atoms and optionally containing a heteroatom selected from O, S(O)ₓ, or NR⁵⁰ and which is optionally substituted;
R¹⁴ is independently selected from hydrogen, alkyl, arylalkyl, cycloalkylalkyl, heteroarylalkyl, heterocyclylalkyl and halo, wherein alkyl, arylalkyl, cycloalkylalkyl, heteroarylalkyl and heterocyclylalkyl are optionally substituted one or more times.
R¹⁶ is selected from cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, heterocycloalkyl fused heteroarylalkyl, (i) and (ii): 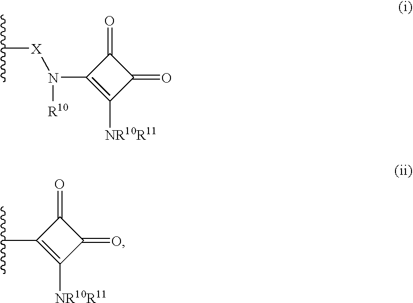
wherein cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl are optionally substituted one or more times;
R²⁰ is selected from hydrogen and alkyl, wherein alkyl is optionally substituted;
R²¹ is a bicyclic or tricyclic fused ring system, wherein at least one ring is partially saturated, and
wherein R²¹ is optionally substituted one or more times, or
wherein R²¹ is optionally substituted by one or more R⁹ groups;
R²³ is selected from hydrogen, hydroxy, halo, alkyl, cycloalkyl, alkoxy, alkenyl, alkynyl, aryl, heteroaryl, NO₂, NR¹⁰R¹¹, CN, SR¹⁰, SSR¹⁰, PO₃R¹⁰, NR¹⁰NR¹⁰R¹¹, NR¹⁰N═CR¹⁰R¹¹, NR¹⁰SO₂R¹¹, C(O)NR¹⁰R¹¹, C(O)OR¹⁰, and fluoroalkyl, wherein alkyl, cycloalkyl, alkoxy, alkenyl, alkynyl, and fluoroalkyl are optionally substituted one or more times;
R³⁰ is selected from alkyl and (C₀-C₆)-alkyl-aryl, wherein alkyl and aryl are optionally substituted;
R⁵⁰ in each occurrence is independently selected from hydrogen, alkyl, aryl, heteroaryl, C(O)R⁸⁰, C(O)NR⁸⁰R⁸¹; SO₂R⁸⁰ and SO₂NR⁸⁰R⁸¹, wherein alkyl, aryl, heteroaryl, C(O)R⁸⁰, C(O)NR⁸⁰R⁸¹, SO₂R⁸⁰ and SO₂NR¹⁰R⁸¹ are optionally substituted;
R⁸⁰ and R⁸¹ in each occurrence are independently selected from hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted, or R⁸⁰ and R⁸¹ when taken together with the nitrogen to which they are attached complete a 3- to 8-membered ring containing carbon atoms and optionally a heteroatom selected from O, S(O)ₓ, —NH, and —N(alkyl) and which is optionally substituted;
E is selected from a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—, —CH₂—W¹— and 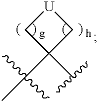
Lₐ is independently selected from CR⁹ and N;
L_{b} is independently selected from C and N with the provisos that both L_{b} are not N, and that the bond between L_{b} and L_{b} is optionally a double bond only if both are L_{b} are carbon;
L_{c} is selected from C and N;
Qₓ is selected from NO₂, CN, SO₂NR¹R², S(O)ₓR¹, SO₃H, C(O)OR¹, NR¹SO₂R¹, OC(O)R¹, OR¹, OR²¹ and 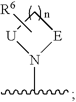
Q_{y} is selected from NR¹R², NR²⁰R²¹ and OR¹;
W is a 5- or 6-membered ring selected from cycloalkyl, heterocycloalkyl, aryl and heteroaryl, wherein cycloalkyl, heterocycloalkyl, aryl and heteroaryl are optionally substituted one or more times with R⁴;
U is selected from C(R⁵R¹⁰), NR⁵, O, S, S═O and S(═O)₂;
W¹ is selected from O, NR⁵, S, S═O, S(═O)₂, N(R¹⁰)(C═O), N(R¹⁰)S(═O)₂ and S(═O)₂N(R¹⁰);
X is selected from a bond and (CR¹⁰R¹¹)_{w}E(CR¹⁰R¹¹)_{w};
g and h are independently selected from 0-2;
n is selected from 0-3;
w is independently selected from 0-4;
x is selected from 0 to 2;
y is selected from 1 and 2;
the dotted line optionally represents a double bond; and
N-oxides, pharmaceutically acceptable salts, prodrugs, formulation, polymorphs, tautomers, racemic mixtures and stereoisomers thereof.

2. A compound according to claim 1, selected from: 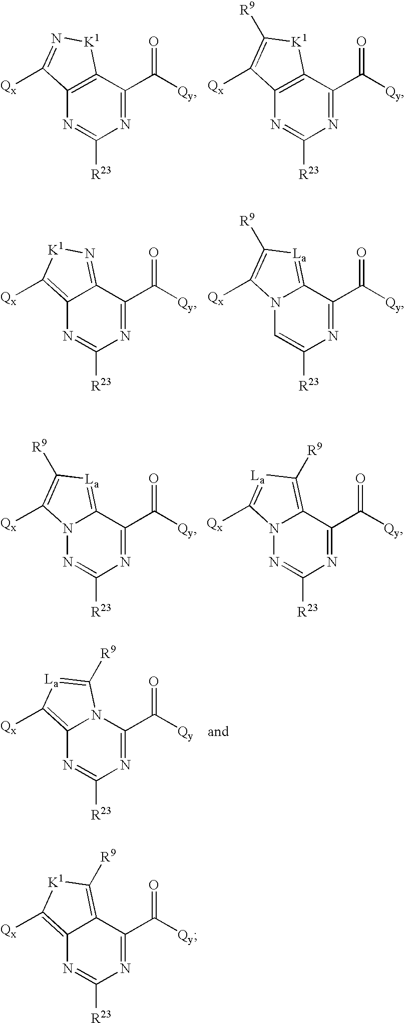
wherein:
Q_{y} is selected from NR¹R² and NR²⁰R²¹;
K¹ is O, S(O)ₓ, or NR⁵¹; and
R⁵¹ is independently selected from hydrogen, alkyl, aryl, heteroaryl, arylalkyl, cycloalkylalkyl, heteroarylalkyl and haloalkyl, wherein alkyl, aryl, heteroaryl, arylalkyl, cycloalkylalkyl, heteroarylalkyl and haloalkyl are optionally substituted one or more times.

3. A compound according to claim 2, selected from:

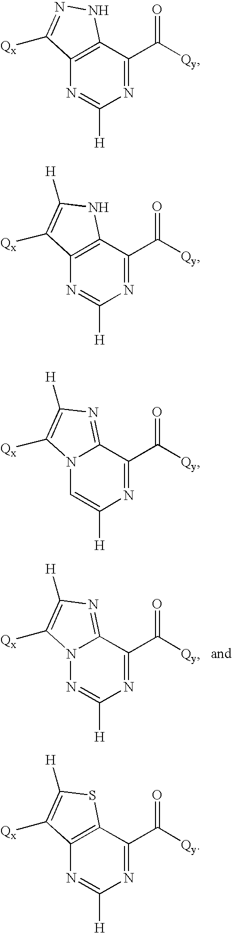

4. The compound according to claim 3, wherein:
Qₓ=SO₂NR¹R², SO₂NR²R², S(O)ₓR¹, C(O)OR¹, NR²SO₂R¹, OC(O)R¹ and OR¹; and
the R¹ in Q_{y} is selected from: 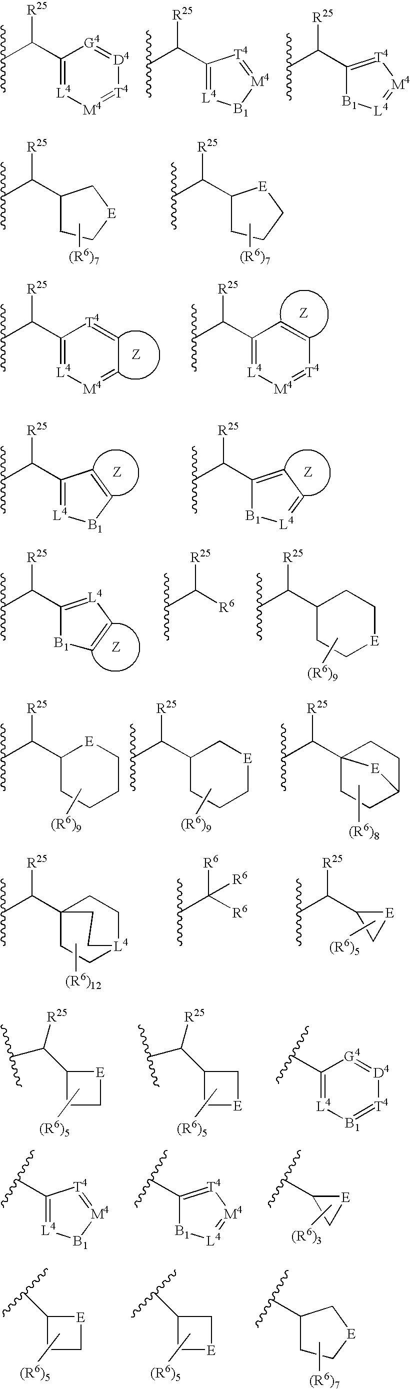 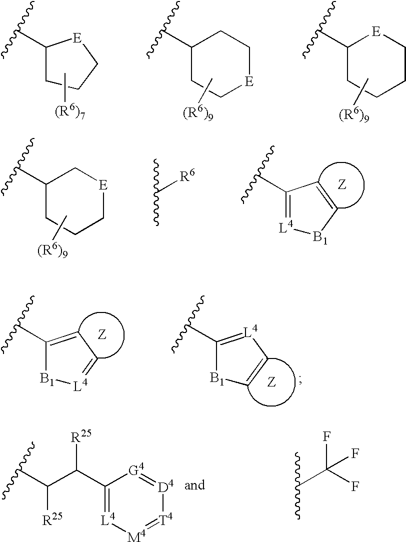
wherein:
R⁹ is independently selected from hydrogen, alkyl, halo, CHF₂, CF₃, OR¹⁰, NR¹⁰R¹¹, NO₂, and CN, wherein alkyl is optionally substituted one or more times;
R²⁵ is independently selected from hydrogen, alkyl, cycloalkyl, C(O)R¹⁰, C(O)NR¹⁰R¹¹ and haloalkyl, wherein alkyl, cycloalkyl, and haloalkyl are optionally substituted one or more times;
B₁ is selected from the group consisting of NR¹⁰, O and S(O)ₓ;
D⁴, G⁴, L⁴, M⁴, and T⁴, are independently selected from CR⁶ and N;
Z is a 5- to 8-membered ring consisting of cycloalkyl, heterocycloalkyl, aryl and heteroaryl, wherein cycloalkyl, heterocycloalkyl, aryl and heteroaryl are optionally substituted one or more times.

5. The compound according to claim 4, wherein:
Qₓ=SO₂NR¹R², SO₂NR²R², S(O)ₓR¹, NR²SO₂R¹; and
the R¹ in Q_{y} is selected from

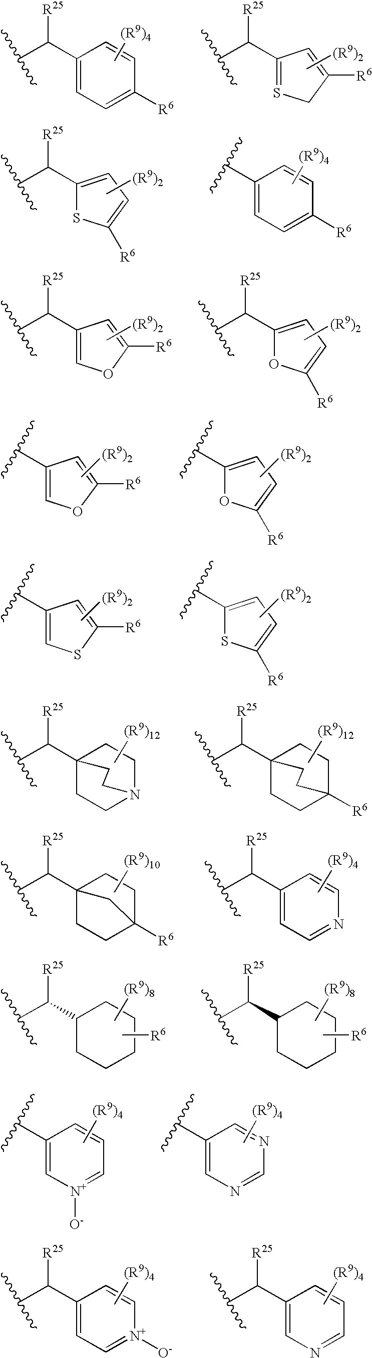

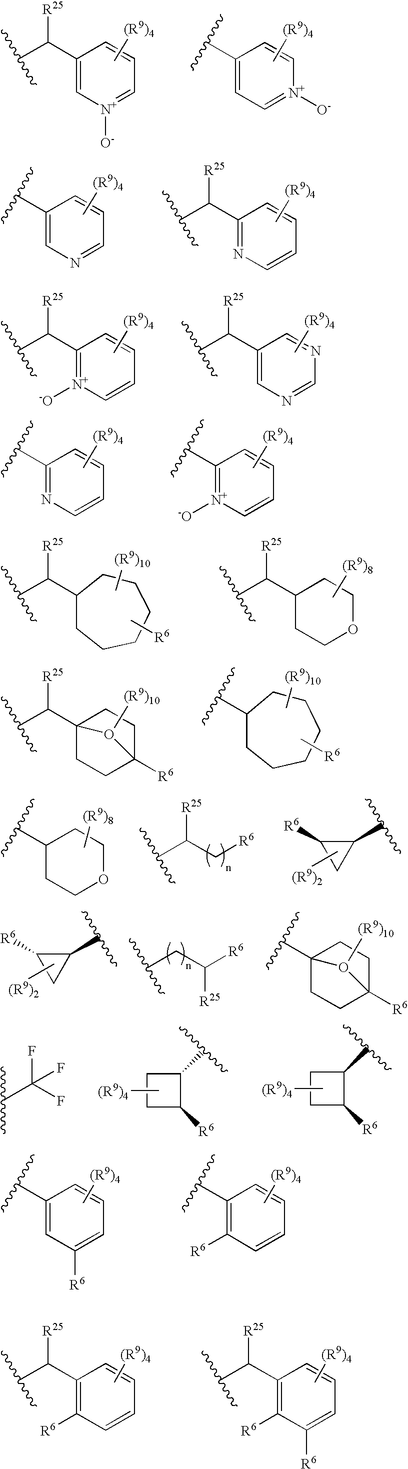

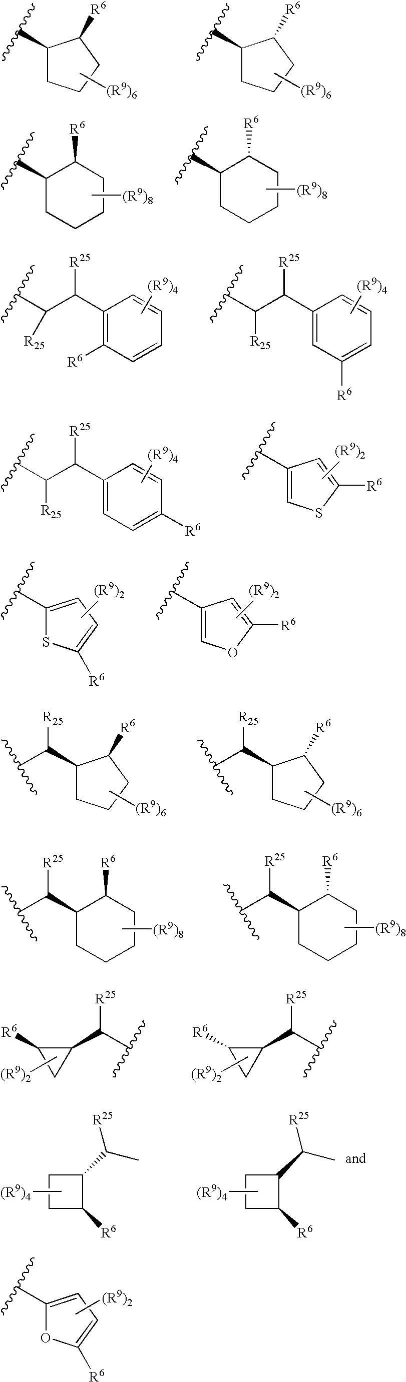

6. The compound according to claim 5 wherein:
R⁶ is selected from hydrogen, halo, CN, OH, CH₂OH, CF₃, CHF₂, OCF₃, OCHF₂, SO₂CH₃, SO₂CF₃, SO₂NH₂, SO₂NHCH₃, SO₂N(CH₃)₂, NH₂, NHCOCH₃, NHCONH₂, NHSO₂CH₃, alkoxy, alkyl alkynyl, CO₂H, 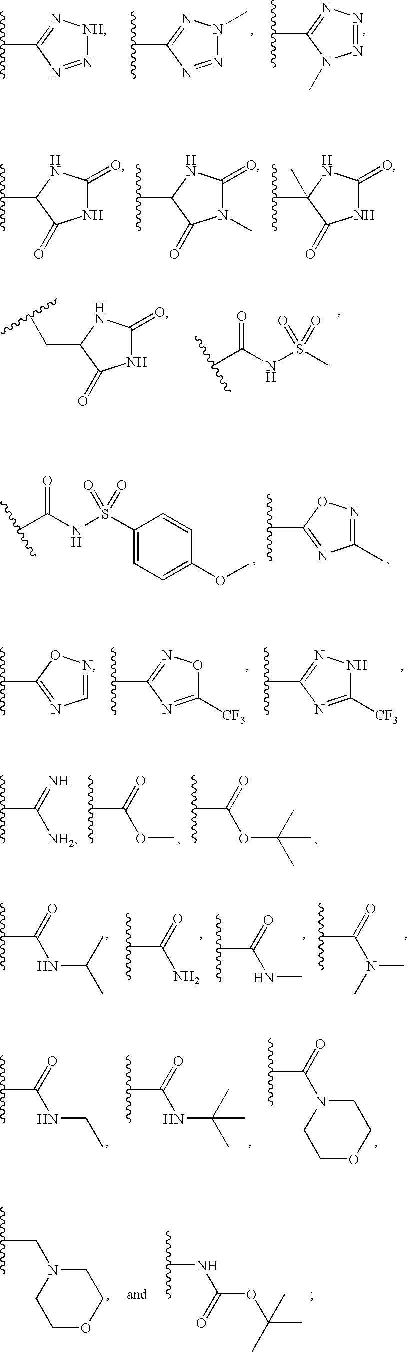
R⁹ is independently selected from hydrogen, fluoro, chloro, CH₃, CF₃, CHF₂, OCF₃, OCH₃ and OCHF₂; and
R²⁵ is selected of hydrogen, CH₃, COOMe, COOH, CONH₂, CONHMe and CON(Me)₂.

7. The compound according to claim 4, wherein:
Qₓ=SO₂NR¹R², SO₂NR²R², S(O)ₓR¹, C(O)OR¹, NR²SO₂R¹, OC(O)R¹ and OR¹; and
the R¹ in Q_{y} is selected from:

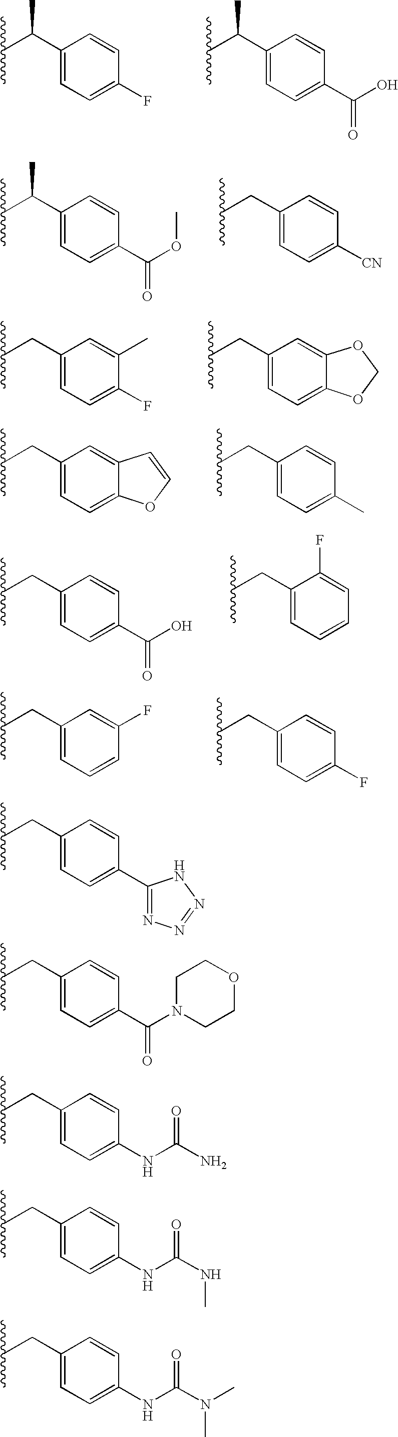

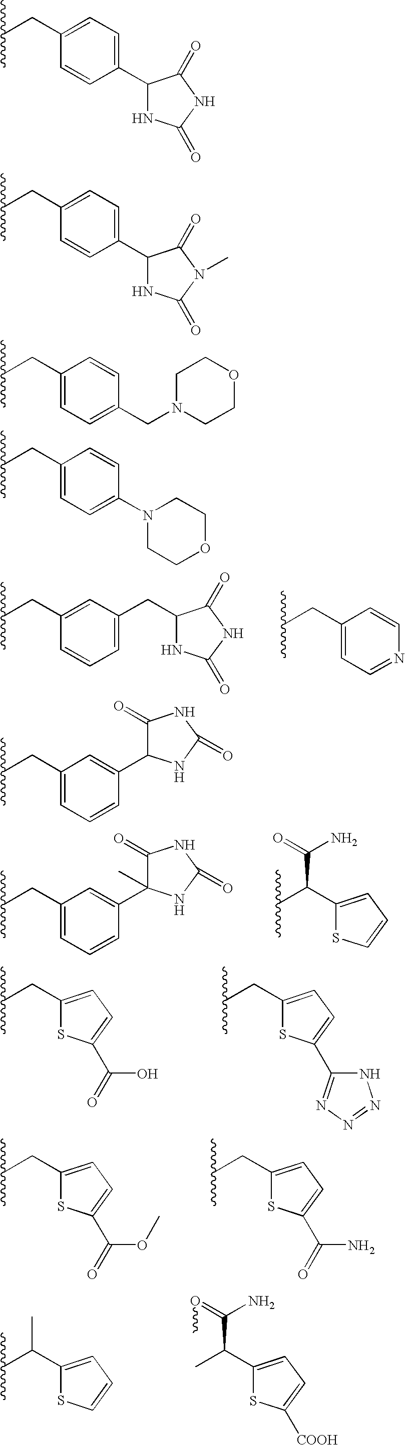

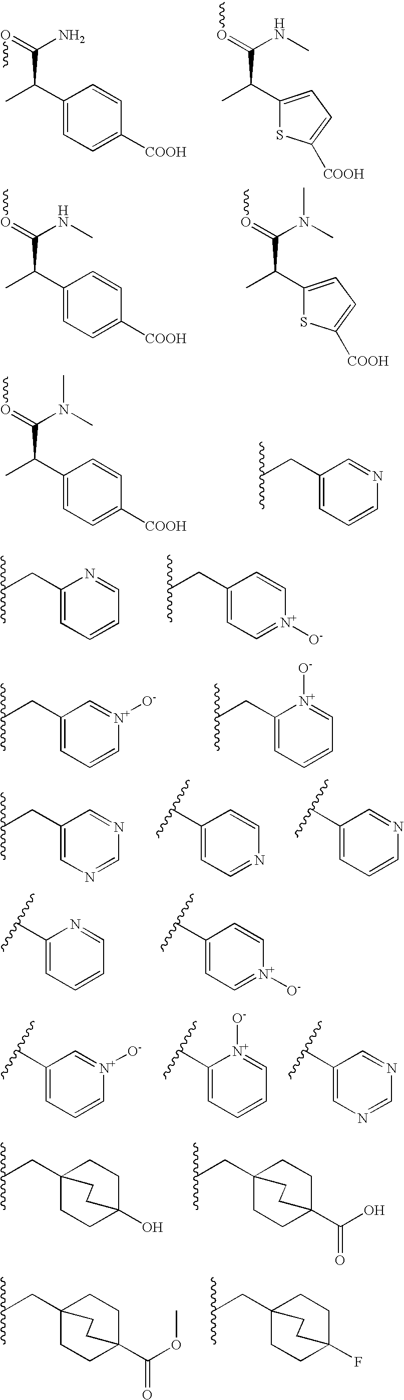

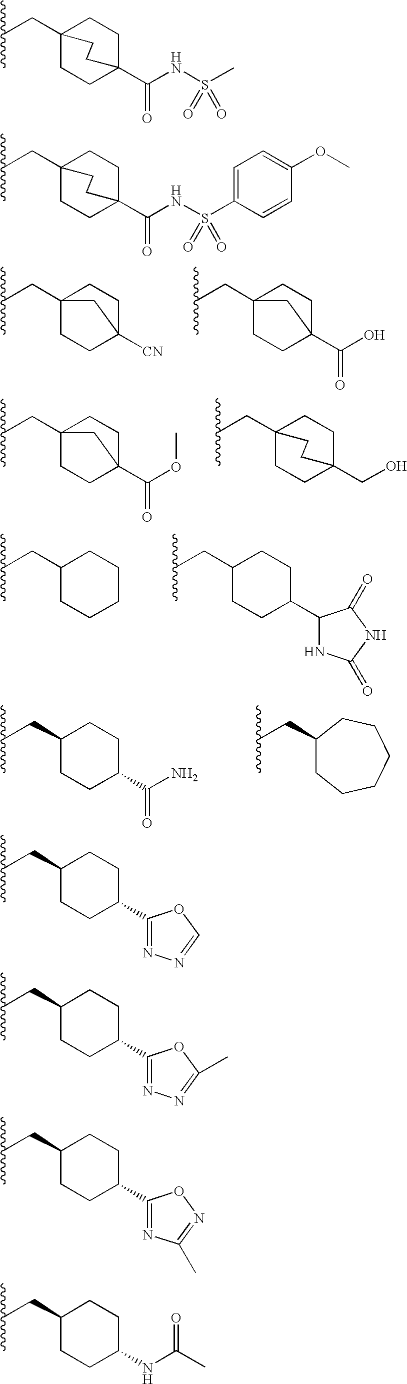

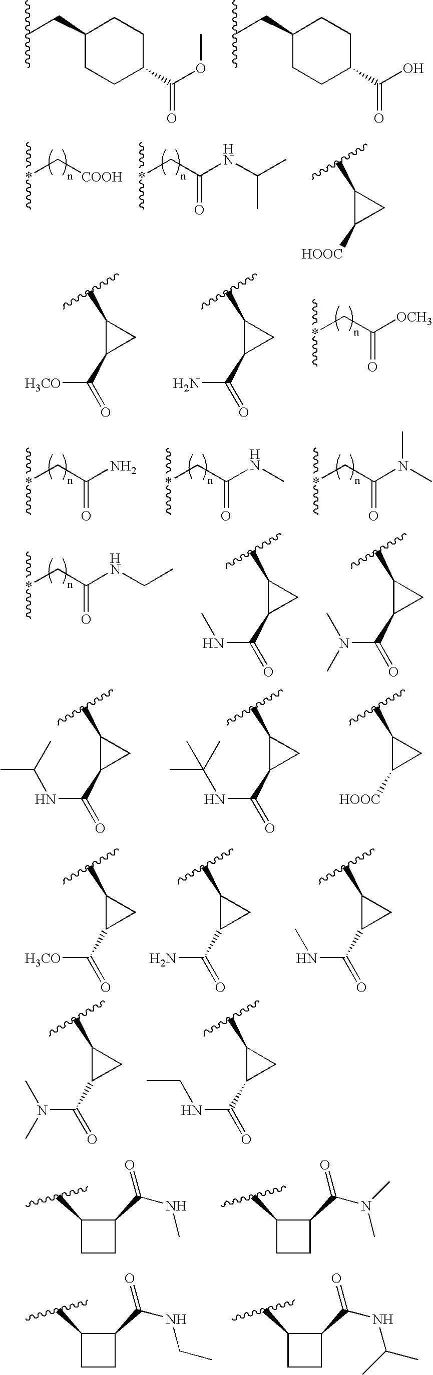

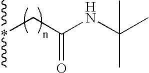

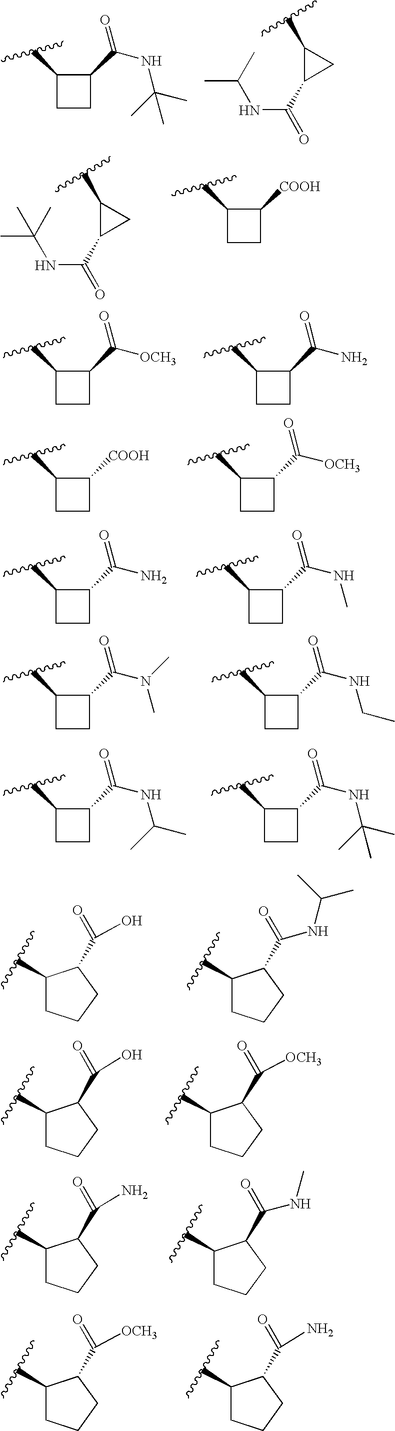

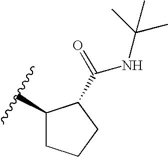

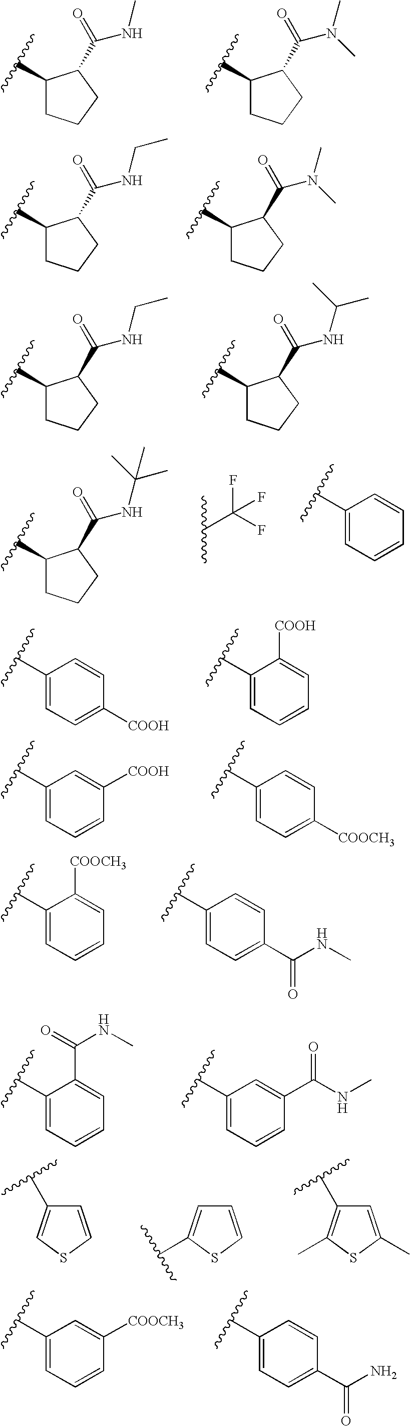

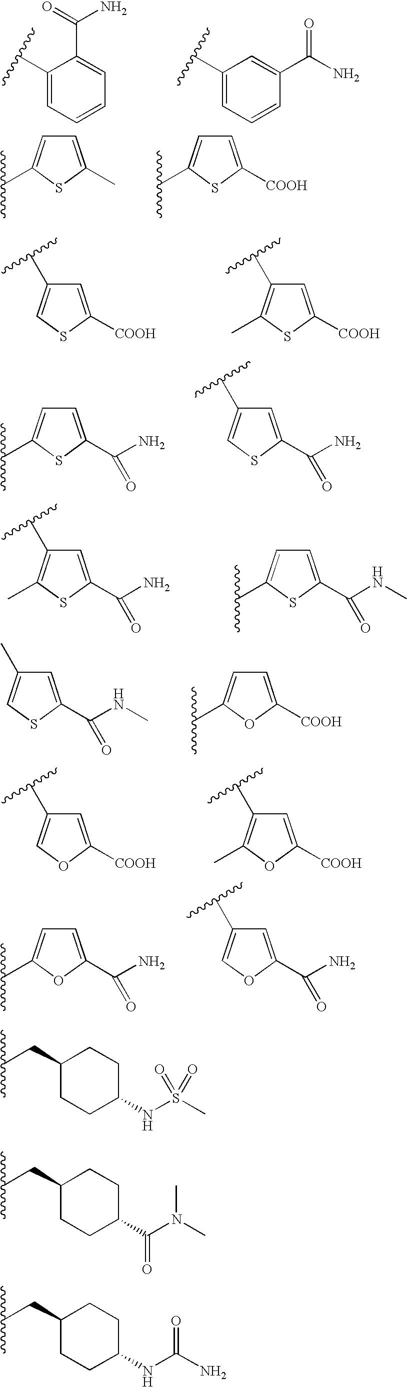

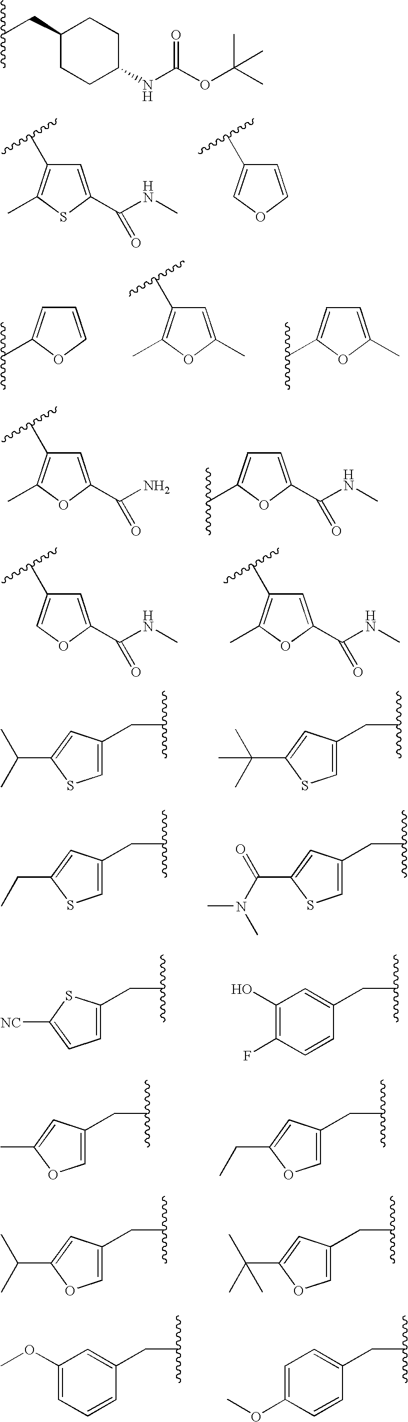

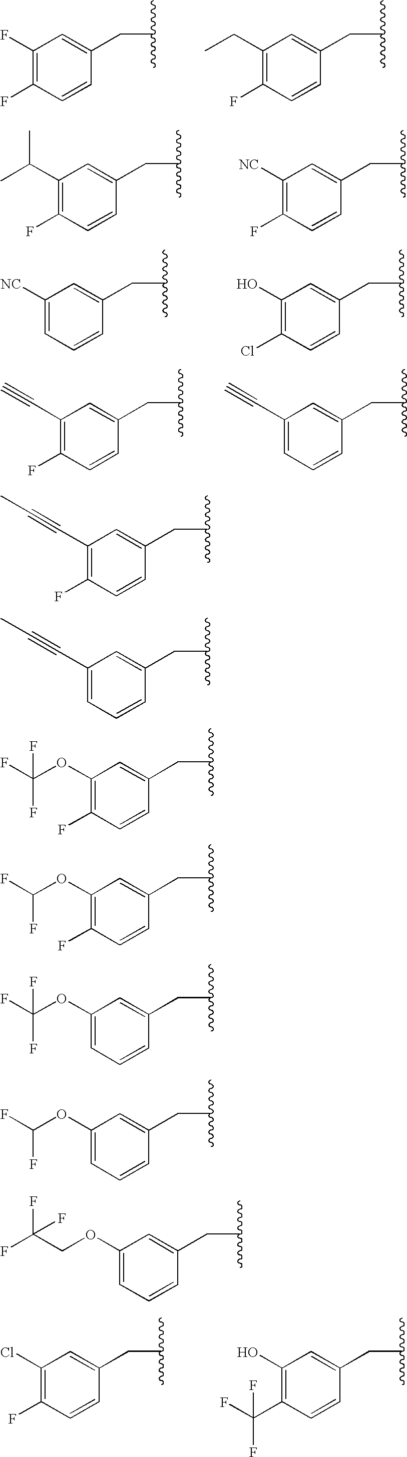

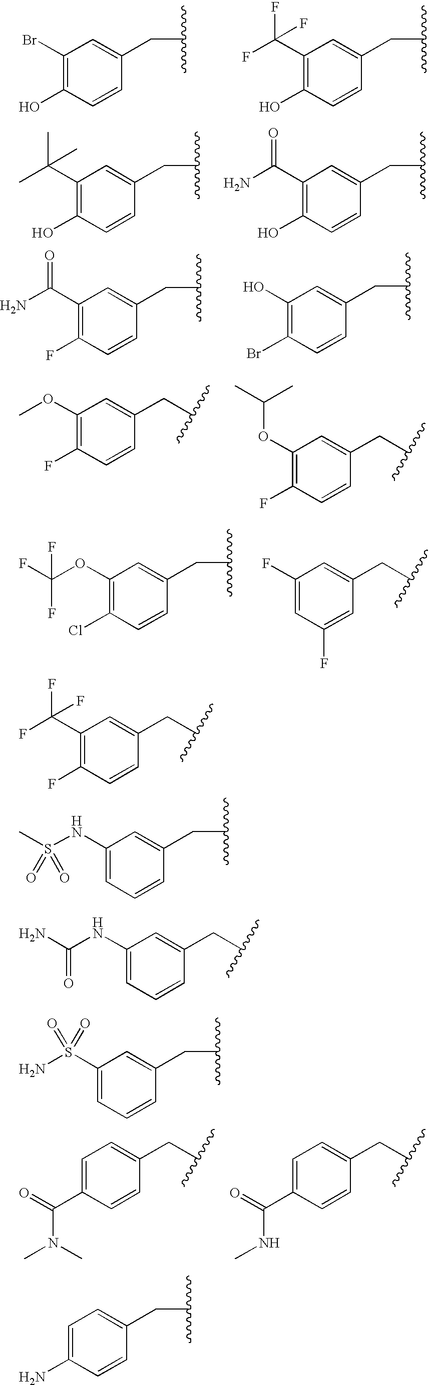

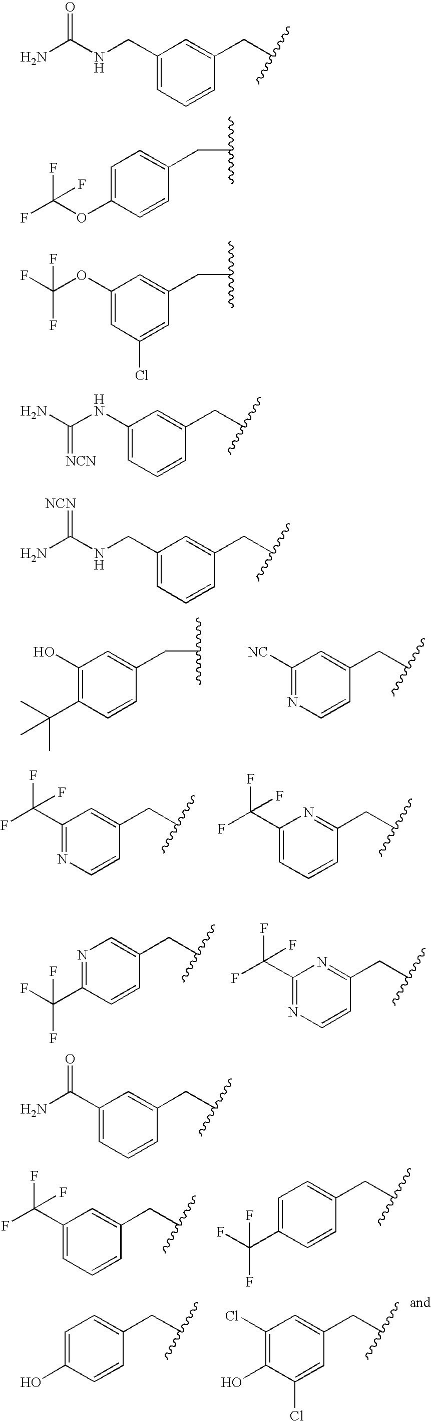

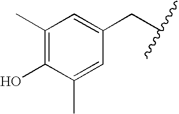

8. The compound according to claim 4 wherein:
Q_{y}=NR¹R²; and
the R¹ of Qy is selected from: 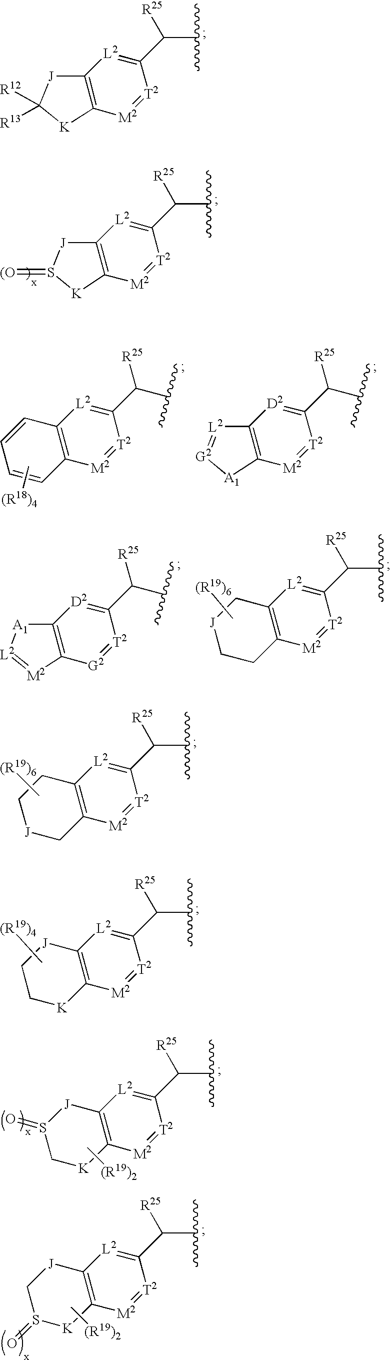 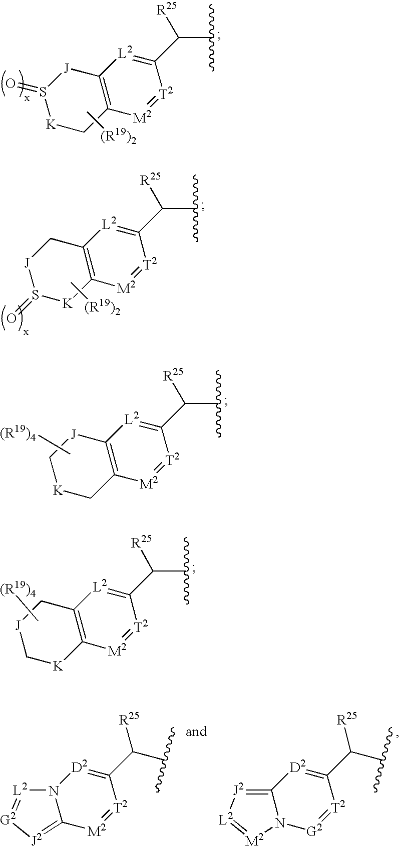
wherein:
R¹² and R¹³ are independently selected from hydrogen, alkyl and halo, wherein alkyl is optionally substituted one or more times, or optionally R¹² and R¹³ together form ═O, ═S or ═NR¹⁰;
R¹⁸ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times;
R¹⁹ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times, or optionally two R¹⁹ groups together at one carbon atom form ═O, ═S or ═NR¹⁰;
R²⁵ is selected from hydrogen, alkyl, cycloalkyl, C(O)NR¹⁰R¹¹ and haloalkyl, wherein alkyl, cycloalkyl, and haloalkyl are optionally substituted one or more times;
J and K are independently selected from CR¹⁰R¹⁸, NR¹⁰, O and S(O)ₓ;
A₁ is selected from NR¹⁰, O and S;
D², G², J², L², M² and T² are independently selected from CR¹⁸ and N.

9. A compound according to claim 8, wherein:
Q_{y}═NR¹R²; and
the R¹ of Q_{y} is selected from:

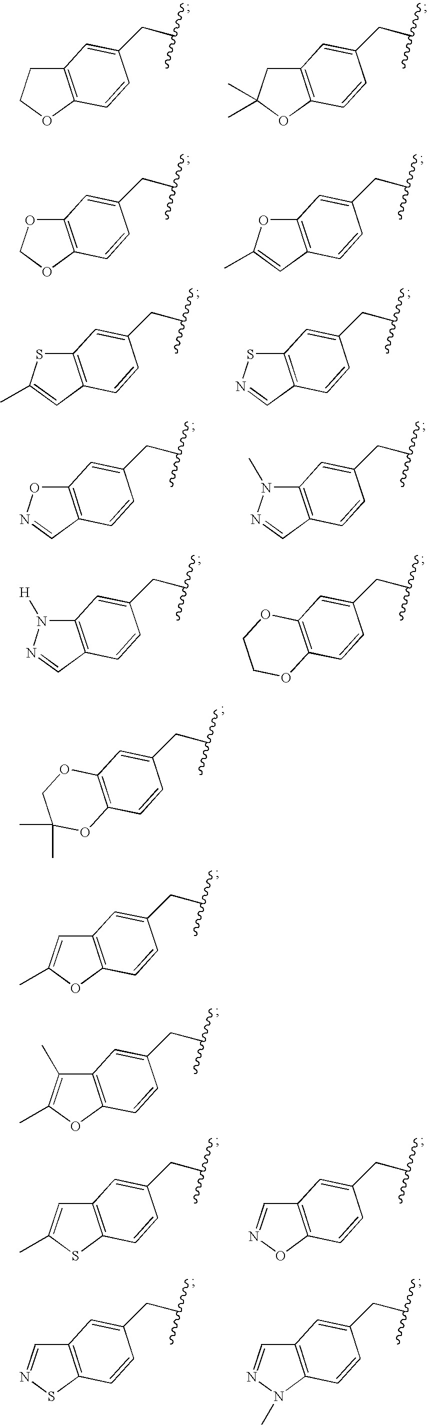

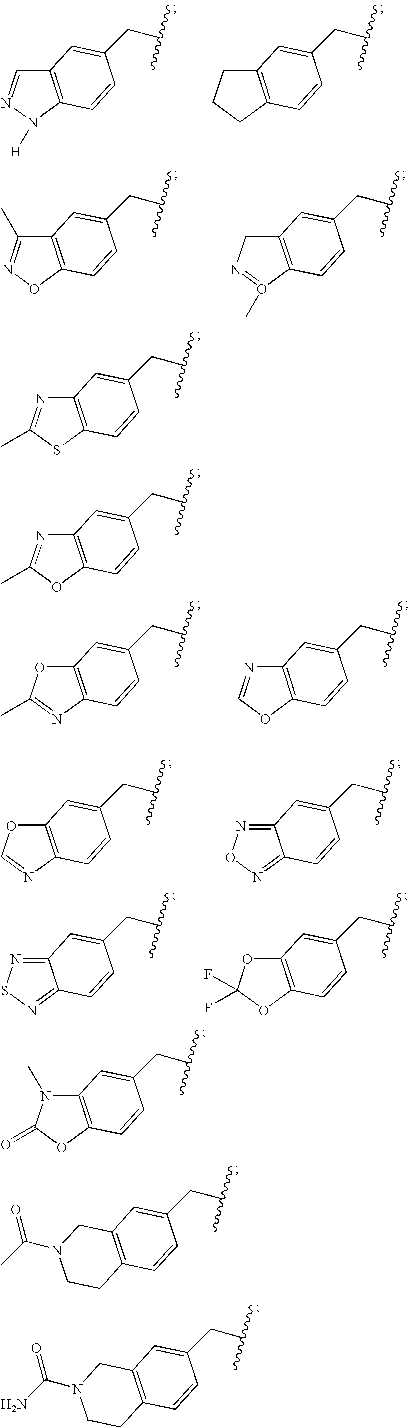

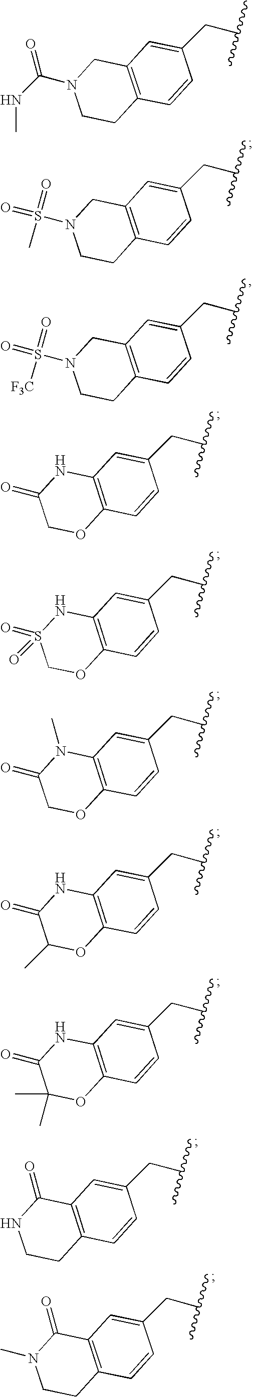

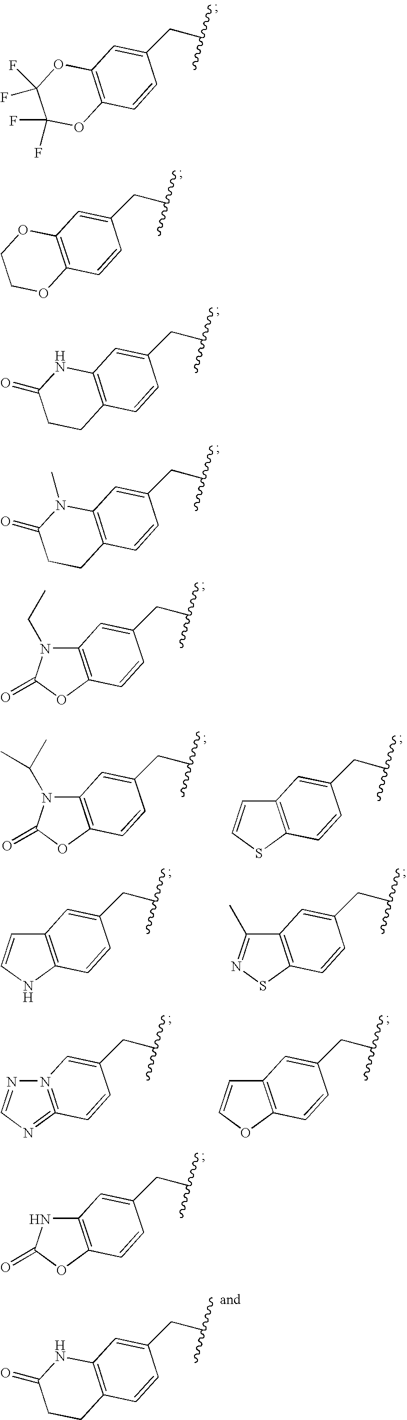

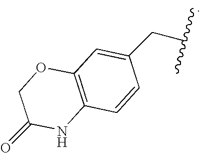

10. The compound according to claim 1, wherein:
Q_{y}=NR¹R²; and
the R¹ of Q_{y} is selected from: 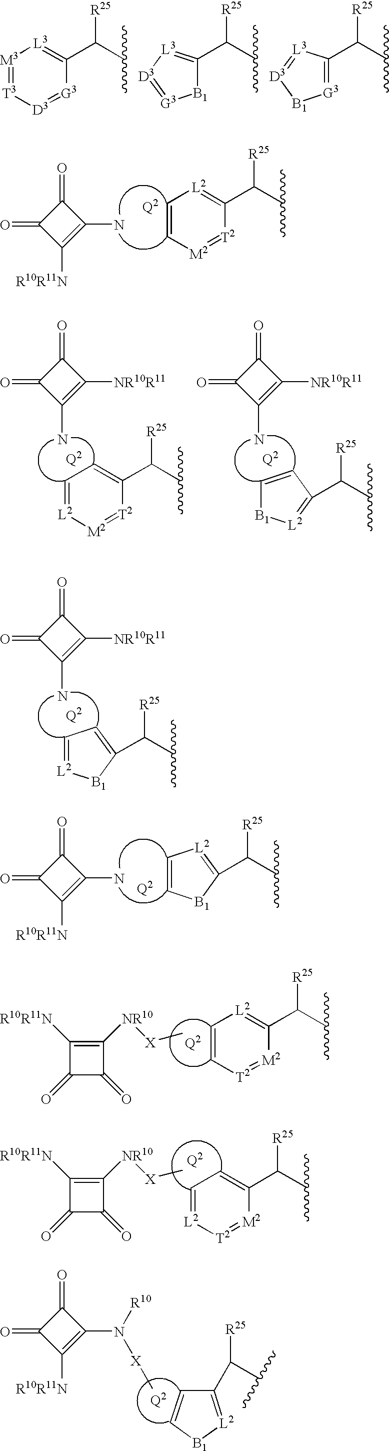 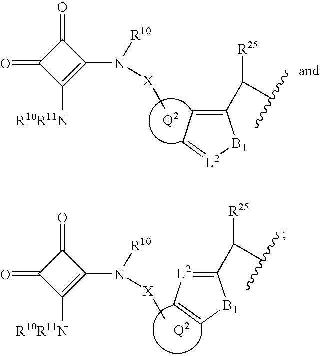
wherein:
R⁵ is independently selected from hydrogen, alkyl, C(O)NR¹⁰R¹¹, aryl, arylalkyl, SO₂NR¹⁰R¹¹ and C(O)OR¹⁰ wherein alkyl, aryl and arylalkyl are optionally substituted one or more times;
R¹⁸ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, —OR¹⁰, OCF₃, OCHF₂, NR¹⁰CONR¹⁰R¹¹, NR¹⁰COR¹¹, NR¹⁰SO₂R¹⁰, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times;
R¹⁹ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times, or optionally two R¹⁹ groups together at one carbon atom form ═O, ═S or ═NR¹⁰;
R²⁵ is selected from hydrogen, alkyl, cycloalkyl, CONR¹⁰R¹¹ and haloalkyl, wherein alkyl, cycloalkyl and haloalkyl are optionally substituted one or more times;
L², M², and T² are independently selected from CR¹⁸ and N;
L³, M³, T³, D³, and G³ are independently selected from N, CR¹⁸, (i), or (ii); 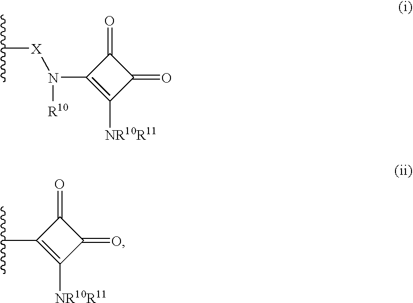
with the provision that one of L³, M³, T³, D³, and G³ is (i) or (ii);
B₁ is selected from the group consisting of NR¹⁰, O and S(O)ₓ;
X is selected from a bond and (CR¹⁰R¹¹)_{w}E(CR¹⁰R¹¹)_{w}
E is selected from a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—, —CH₂—W¹— and 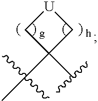
W¹ is selected from O, NR⁵, S, S═O, S(═O)₂, N(R¹⁰)(C═O), N(R¹⁰)S(═O)₂ and S(═O)₂N(R¹⁰);
U is selected from C(R⁵R¹⁰), NR⁵, O, S, S═O, S(═O)₂;
g and h are independently selected from 0-2;
w is selected from 0-4; and
Q² is a 5- to 8-membered ring consisting of cycloalkyl, heterocycloalkyl, aryl, heteroaryl, which is optionally substituted one or more times with R¹⁹.

11. The compound according to claim 10, wherein:
Q_{y}=NR¹R²; and
R¹ is selected from:

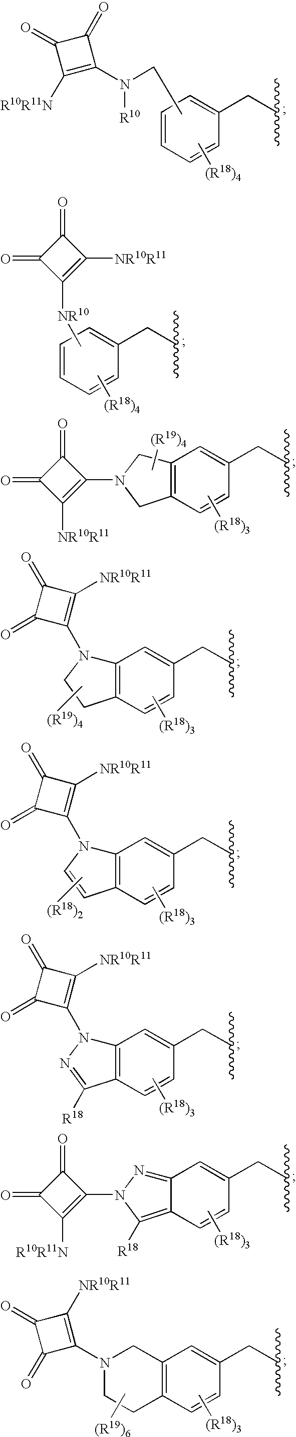

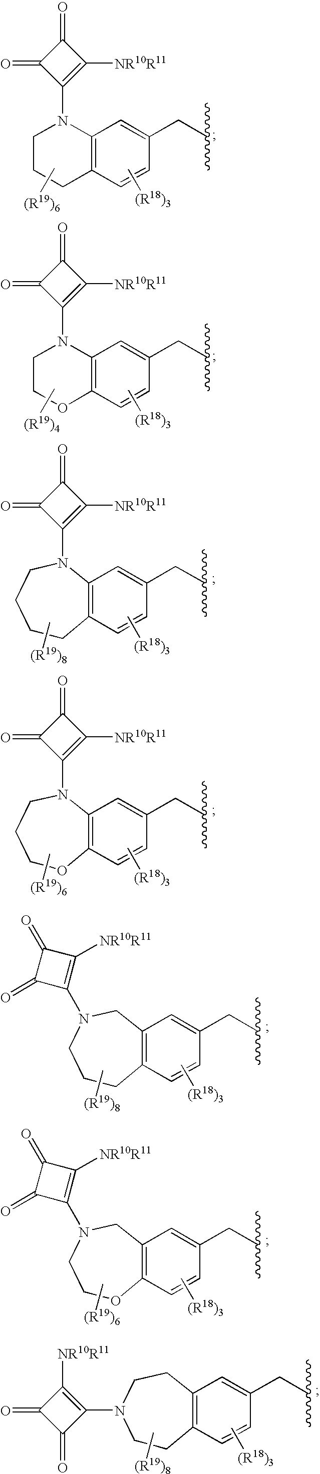

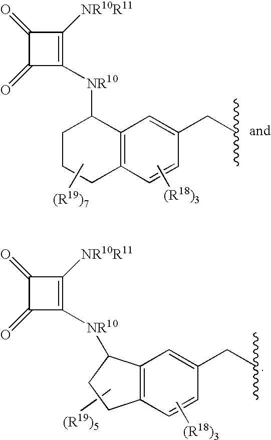

12. The compound according to claim 11, wherein:
Q_{y}=NR¹R²; and
the R¹ of Q_{y} is selected from:

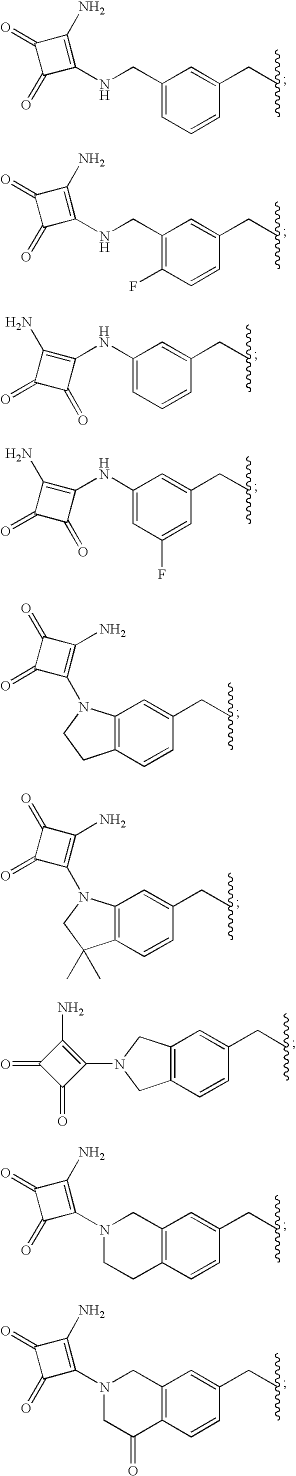

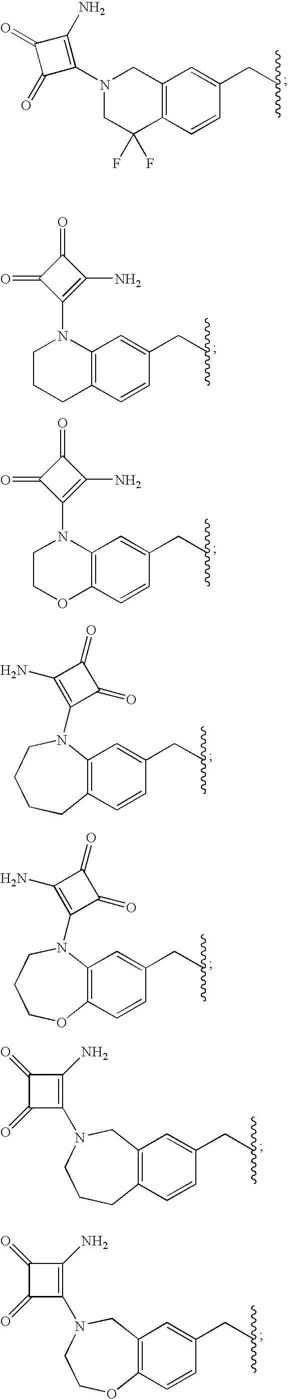

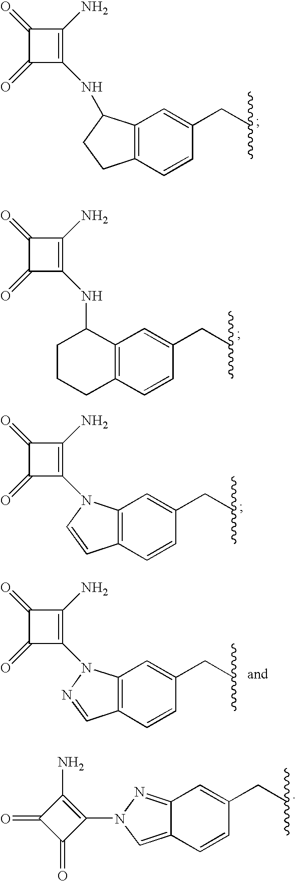

13. A compound according to claim 1, wherein said compound is selected from:

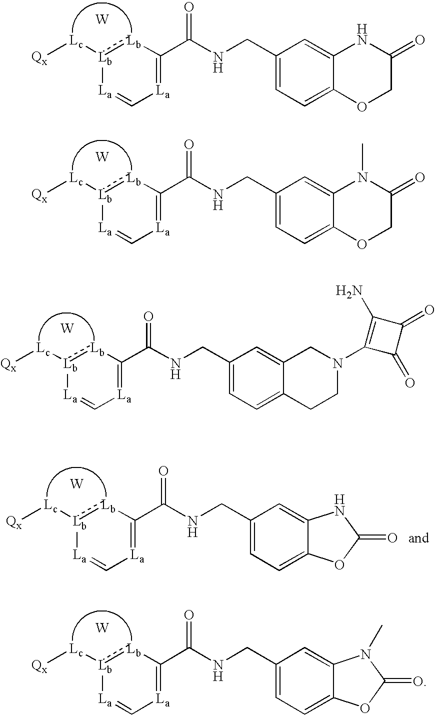

14. A compound according to claim 1 selected from: 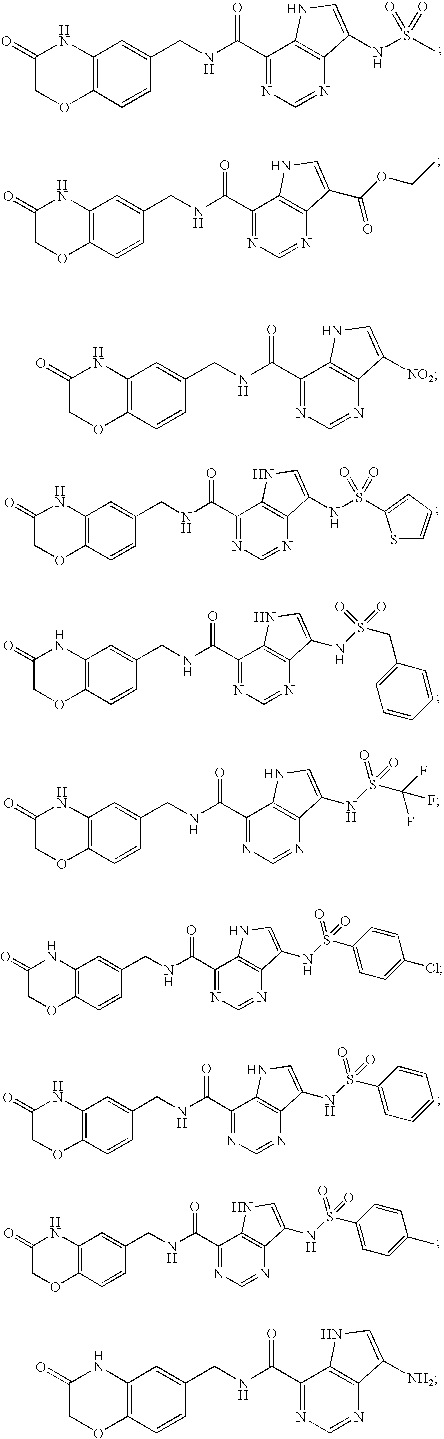 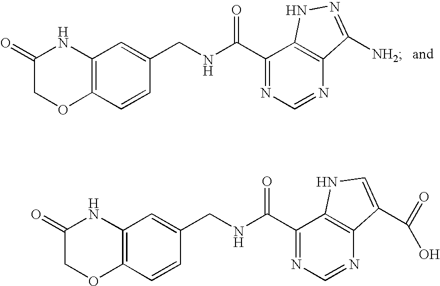
or a pharmaceutically acceptable salt thereof.

15. A pharmaceutical composition comprising an effective amount of a compound according to claim 1 and a pharmaceutically acceptable carrier.

16. A method of treating a metalloprotease mediated disease, comprising administering to a subject in need of such treatment an effective amount of a compound according to claim 1.

17. The method according to claim 16, wherein the disease is selected from rheumatoid arthritis, osteoarthritis, inflammation, atherosclerosis and multiple sclerosis.

18. A pharmaceutical composition comprising:
a) an effective amount of a compound according to claim 1;
b) a pharmaceutically acceptable carrier; and
c) a member selected from: (a) a disease modifying antirheumatic drug; (b) a nonsteroidal anti-inflammatory drug; (c) a COX-2 selective inhibitor; (d) a COX-1 inhibitor; (e) an immunosuppressive; (f) a steroid; (g) a biological response modifier; and (h) a small molecule inhibitor of pro-inflammatory cytokine production.
